(19) United States
(12) Patent Application Publication
(10) Pub. No.: US 2022/0325245 A1
(43) Pub. Date: Oct. 13, 2022

(54) METHODS FOR PRODUCTION OF CAR-NK CELLS AND USE THEREOF

(71) Applicant: Board of Regents, The University of Texas System, Austin, TX (US)

(72) Inventors: Katy Rezvani, Houston, TX (US); Elizabeth Shpall, Houston, TX (US); Enli Liu, Houston, TX (US)

(21) Appl. No.: 17/593,085

(22) PCT Filed: Mar. 25, 2020
(86) PCT No.: PCT/US2020/024671

§ 371 Date: Sep. 8, 2021

Related U.S. Application Data
(60) Provisional application No. 62/826,856, filed on Mar. 29, 2019.

(51) Int. Cl. C12N 5/0783; A61K 35/17; A61P 35/00

(52) U.S. Cl. CPC C12N 5/0646; A61K 35/17; A61P 35/00; C12N 2501/2302; C12N 2502/1121; C12N 2510/00

(57) ABSTRACT

Provided herein are methods for expanding NK cells expressing chimeric antigen receptors and/or T cell receptors. Further provided are methods for treating diseases by administering the CAR NK cells.

This application is a national phase application under 35 U.S.C. § 371 that claims priority to International Application No. PCT/US2020/024671 filed Mar. 25, 2020, which claims priority to U.S. Provisional Patent Application Ser. No. 62/826,856, filed Mar. 29, 2019, both of which are incorporated by reference herein in their entirety.

### INCORPORATION OF SEQUENCE LISTING

The instant application contains a Sequence Listing which has been submitted electronically in ASCII format and is hereby incorporated by reference in its entirety. Said ASCII copy, created on Sep. 9, 2021, is named SeqLst_UTSC_P1186.txt and is 2,436 bytes in size.

### BACKGROUND

### 1. Field

The present invention relates generally to the fields of immunology and medicine. More particularly, it concerns methods of expanding natural killer (NK) cells.

### 2. Description of Related Art

Despite technological advancements in the diagnosis and treatment options available to patients diagnosed with cancer, the prognosis still often remains poor and many patients cannot be cured. Immunotherapy holds the promise of offering a potent, yet targeted, treatment for patients diagnosed with various tumors with the potential to eradicate the malignant tumor cells without damaging normal tissues. In theory, the T cells of the immune system are capable of recognizing protein patterns specific for tumor cells and to mediate their destruction through a variety of effector mechanisms. Adoptive T cell therapy is an attempt to harness and amplify the tumor-eradicating capacity of a patient's own T cells and then return these effectors to the patient in such a state that they effectively eliminate residual tumor, however without damaging healthy tissue. Although this approach is not new to the field of tumor immunology, many drawbacks in the clinical use of adoptive T cell therapy impair the full use of this approach in cancer treatments. For example, chimeric antigen receptor T cells (CAR T) cells are patient-specific and have to be produced for each patient on an individual case basis.

On the other hand, cord blood (CB)-derived natural killer (NK) cells provide an off-the-shelf source of cells for immunotherapy and also harness the inherent cytotoxicity of NK cells against many tumors. While studies have been performed on CAR NK cells derived from peripheral blood, these cells are also not ideal for an ‘off-the-shelf’ approach. This is because a donor has to be identified for NK cell donation in each case.

CAR-engineered NK92 cells have also been studied; however, NK92 is an NK cell line derived from a lymphoma patient which lacks many of the NK cell receptors important for NK cell cytotoxicity. In addition, since the cell line was derived from a patient with lymphoma, the cells must be irradiated prior to infusion. This lack of NK cell receptors and need for irradiation significantly impairs the ability of the cells to proliferate and persist, making them less effective than CAR-modified CB-NK cells that express the full array of NK cell receptors. Thus, there is an unmet need for methods of generating CAR NK cells with high efficiency for use in clinical therapies.

### SUMMARY

In one embodiment, the present disclosure provides methods and compositions related to therapies for a medical condition, such as cancer. In particular embodiments, the methods and compositions concern immunotherapies and/or cell therapies. In a specific embodiment, the disclosure concerns an ex vivo method for producing natural killer (NK) cells engineered to express a chimeric antigen receptor (CAR) and/or T cell receptor (TCR) comprising culturing a starting population of NK cells in the presence of artificial presenting cells (APCs) or other feeder cells and at least one cytokine; introducing a CAR and/or TCR expression vector into the NK cells; and expanding the NK cells in a gas-permeable bioreactor in the presence of APCs and at least one cytokine, thereby obtaining an expanded population of engineered NK cells. In some aspects, the gas permeable bioreactor is G-Rex®100M. In certain aspects, the method does not comprise performing HLA matching. In some alternative cases, any or all steps of the method occur in the absence of a gas-permeable bioreactor.

In some aspects, the engineered NK cells express a CAR. In certain aspects, the engineered NK cells express a TCR. In particular aspects, the engineered NK cells express a CAR and TCR or multiple antigen receptors. In particular aspects, the population of engineered NK cells are GMP-compliant. In particular aspects, the complete method is performed in less than 2 weeks, such as 8 days, 9 days, 10 days, 11, days, 12 days, 13 days, or 14 days. In other aspects, the complete method may take 3, 4 or more weeks. In some aspects, the NK cells are allogeneic with respect to an individual. In other aspects, the NK cells are autologous with respect to an individual.

In some aspects, the starting population of NK cells is obtained from cord blood, peripheral blood, bone marrow, CD34⁺ cells, or iPSCs. In particular aspects, the starting population of NK cells is obtained from cord blood. In some aspects, the cord blood has previously been frozen. In certain aspects, the starting population of NK cells is obtained by isolating mononuclear cells using a ficoll-paque density gradient. In some aspects, the method further comprises depleting the mononuclear cells of CD3, CD14, and/or CD19 cells to obtain the starting population of NK cells. In some aspects, the method further comprises depleting the mononuclear cells of CD3, CD14, and CD19 cells to obtain the starting population of NK cells. In particular aspects, depleting comprises performing magnetic sorting. In other aspects, NK cells could be positively selected using sorting, magnetic bead selection or other methods to obtain the starting populations of NK cells.

In certain aspects, the APCs are gamma-irradiated APCs. In some aspects, the APCs are universal APCs (uAPCs). In some aspects, the uAPCs are engineered to express (1) CD48 and/or CS1 (CD319), (2) membrane-bound interleukin-21 (mbIL-21), and (3) 41BB ligand (41BBL). In particular aspects, the NK cells and APCs are present at a 1:1 to 1:100 ratio, such as a 1:1, 1:2, 1:3, 1:4, 1:5, 1:6, 1:7, 1:8, 1:9, or 1:10 ratio. In certain aspects, the NK cells and APCs are present at a 1:2 ratio.

In some aspects, at least one cytokine is IL-2, IL-21, IL-15, or IL-18. In certain aspects, the culturing and/or expanding of the NK cells is in the presence of 2, 3, or 4 cytokines. In some aspects, the cytokines are selected from the group consisting of IL-2, IL-21, IL-15, and IL-18. In some aspects, at least one cytokine, such as IL-2, is present at a concentration of 100-300 U/mL, such as 100, 125, 150, 175, 200, 225, 250, 275, or 300 U/mL. In certain aspects, the at least one cytokine is present at a concentration of 200 U/mL.

In certain aspects, introducing the CAR and/or TCR comprises transduction or electroporation. In some aspects, the transduction is retronectin transduction. In particular aspects, the transduction has an efficiency of at least 20%, such as at least 25%, 30%, 35%, 40%, 45%, 50%, 55%, 60%, 65%, 66%, 67%, 68%, 69%, 70%, 71%, 72%, 73%, 74%, 75%, or higher. In some aspects, the CAR and/or TCR expression construct is a lentiviral vector or retroviral vector. In certain aspects, the method results in at least 1000-fold expansion, such as at least 1100-, 1200-, 1300-, 1400-, 1500-, 1600-, 1700-, 1800-, 1900-, 2000-, 2100-, 2200-, 2300-, 2400-, 2500-fold or higher expansion.

In some aspects, the CAR and/or TCR has antigenic specificity for CD19, CD319/CS1, BCMA, CD38, CLL1, CD70, ROR1, CD20, CD5, CD70, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, and/or VEGFR2. In some aspects, the CAR and/or expression construct further expresses a cytokine or 2, 3, or 4 cytokines. In certain aspects, the cytokine is IL-15, IL-21, IL-18, or IL-2.

In additional aspects, the method further comprises cryopreserving the population of engineered NK cells. In some aspects, the engineered NK cells are cryopreserved. Further provided herein is a population of cryopreserved NK cells.

In another embodiment, there is provided a population of engineered NK cells produced according to the methods of present embodiments. Further provided herein is a pharmaceutical composition comprising the population of engineered NK cells of the embodiments and a pharmaceutically acceptable carrier. Another embodiment provides a composition comprising an effective amount of the engineered NK cells of the embodiments for use in the treatment of a disease or disorder in a subject. Also provided herein is the use of a composition comprising an effective amount of the engineered NK cells of the embodiments for the treatment of an immune-related disorder in a subject.

A further embodiment provides a method of treating an immune-related disorder in a subject comprising administering an effective amount of engineered NK cells of the embodiments to the subject. In certain aspects, the method does not comprise performing HLA matching. In particular aspects, the NK cells are KIR-ligand mismatched between the subject and donor. In specific aspects, the method does not comprise performing HLA matching. In particular aspects, the absence of HLA matching does not result in graft versus host disease or toxicity.

In some aspects, the immune-related disorder is a cancer, autoimmune disorder, graft versus host disease, allograft rejection, or inflammatory condition. In certain aspects, the immune-related disorder is an inflammatory condition and the immune cells have essentially no expression of glucocorticoid receptor. In some aspects, the subject has been or is being administered a steroid therapy. In some aspects, the NK cells are autologous. In certain aspects, the NK cells are allogeneic.

In particular aspects, the immune-related disorder is a cancer. In some aspects, the cancer is a solid cancer or a hematologic malignancy.

In additional aspects, the method further comprises administering at least a second therapeutic agent. In some aspects, the at least a second therapeutic agent comprises chemotherapy, immunotherapy, surgery, radiotherapy, or biotherapy. In particular aspects, the NK cells and/or at least a second therapeutic agent are administered intravenously, intraperitoneally, intratracheally, intrathecally, intratumorally, intramuscularly, endoscopically, intralesionally, percutaneously, subcutaneously, regionally, or by direct injection or perfusion. The combination therapies may be administered sequentially or simultaneously.

A further embodiment provides a method of treating an infection of any kind in a subject comprising administering an effective amount of engineered NK cells of the embodiments to the subject. In certain aspects, the method does not comprise performing HLA matching. In particular aspects, the NK cells are KIR-ligand mismatched between the subject and donor. In specific aspects, the method does not comprise performing HLA matching. In some aspects, the NK cells are KIR-ligand mismatched between the subject and donor. In particular aspects, the absence of HLA matching does not result in graft versus host disease or toxicity. In some aspects, the NK cells are autologous. In certain aspects, the NK cells are allogeneic.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Clinical GMP-grade CAR-NK transduction and expansion.

FIG. 2: Characteristics of GMP-grade CAR-transduced CB-NK cells generated from 5 different CB units after 14 days of culture.

FIG. 3: CAR NK cell expansion in flask versus G-Rex® bioreactor.

FIG. 4: Average survival (days) of mice in groups treated with different NK cell preparations.

FIG. 5: Percent survival of mice engrafted with Raji tumors and treated with different NK cell preparations.

FIG. 6: Comparison of survival of mice engrafted with Raji tumors and treated with different NK cell preparations.

FIG. 7: Biofluorescent imaging of mice treated with indicated NK cell preparations.

FIG. 8: Impact of blocking KIR-HLA interaction on activity of CAR NK cells against tumor targets.

FIG. 9: Table 1. Characteristics of Patients at Baseline.

FIG. 10: Table 2. Adverse Events in the 11 Study Patients.

FIG. 11: Clinical Response to CAR-NK Therapy and Postremission Treatments. Shown are the clinical outcomes and subsequent therapies for the 11 patients who were treated with anti-CD19 chimeric antigen receptor (CAR) natural killer (NK) cells in the study. Responses were confirmed and assessed according to the 2018 criteria of the International Workshop on Chronic Lymphocytic Leukemia and the 2014 Lugano classification for non-Hodgkin's lymphoma. The indicated responses include partial response (PR) and complete response (CR); MRD denotes minimal residual disease, as assessed on multiparameter flow cytometry, with or without bone marrow (BM) infiltration. Patient 3 received four doses (×4) of rituximab; for Patients 5 and 7, the dashed white line indicates the duration of postremission therapy. HSCT denotes hematopoietic stem-cell transplantation.

FIGS. 12A and 12B: Persistence of CAR-NK Cells after Infusion. FIG. 12A shows measurements of CAR-NK cells in peripheral-blood samples, as assessed on quantitative polymerase-chain-reaction assay, according to the dose of CAR-NK cells received by the patient. The horizontal gray line at 3 copies per microgram of DNA represents the lower limit of quantification for this assay. The solid horizontal bars indicate the median copy numbers at the various time points for each dose level. After a single infusion of CAR-NK cells, CAR sequences could be detected in all 11 patients. The values increased and remained detectable in peripheral blood for up to 1 year after infusion, regardless of the dose level. No relationship was observed between the administered cell dose and the CAR-NK copy number beyond day 14 after infusion, which suggests that the persistence of CAR-NK cells was driven by in vivo proliferation of the infused cells. The length of follow-up varied among the patients.

FIG. 12B shows the peak copy numbers of CAR-NK cells in the first 28 days after infusion for the 11 patients, according to their response to therapy. Patients who had a response at day 30 had a significantly higher copy-number peak of CAR-NK cells after the infusion than those who did not have a response (median value, 31,744 vs. 903 copies per microgram; P=0.02). The black horizontal bars indicate median values.

FIGS. 13A-13C: GMP-grade CAR-NK cells kill primary CLL targets in a perforin-dependent manner. FIG. 13A shows lysis of primary CLL targets (n=4) by GMP-grade iC9/CAR19/IL-15 transduced CB NK cells (red line) compared to paired ex vivo expanded non-transduced NK cells (NT-NK cells; black line). *** represents p<0.0001 and **p<0.01.

FIG. 13B represents the fold change in mean fluorescence intensity of perforin (red circles) after treatment with concanomycin A (CMA), calculated as follows: perforin MFI after culture with CMA/perforin MFI after culture with CMA. The MFI levels of CD56 (black circles) and CAR (green circles) on the surface of NK cells were measured as controls and remained unchanged after treatment with CMA (n=3). FIG. 13C shows lysis of primary CLL targets (n=4) by GMP-grade iC9/CAR19/IL-15 transduced CB NK cells before (solid circles) and after (open circles) treatment with CMA.; ** represents p<0.01 and *p<0.05.

FIG. 14: Radiological response in patient 5. FDG PET-CT scans from patient 5 performed at study enrollment, before (upper row) and 29 days after receiving the CAR-NK cell infusion (lower row). Upper right corner projection image showing FDG uptake in nodes above and below the diaphragm. Upper right middle showing FDG PET-CT scan with abnormal uptake in enlarged mesenteric nodes (dark arrow). Upper left middle PET-CT scan showing enlarged mesenteric nodes (light arrow). Upper left “fused” PETCT scan showing FDG uptake localized to mesenteric adenopathy. Lower right corner projection image showing resolution of FDG uptake in nodes above and below the diaphragm. Lower right middle showing FDG PET-CT scan with no uptake in mesenteric nodes (dark arrow). Lower left middle PET-CT scan showing stable enlarged mesenteric nodes (light arrow). Lower left “fused” PET-CT scan showing no FDG uptake in mesenteric adenopathy (arrow).

FIG. 15: Persistence of CAR-NK cells after infusion according to the degree of HLA mismatch between the CB CAR-NK cells and the recipient. The persistence and expansion of iC9/CAR19/IL-15-modified CB-NK cells in peripheral blood samples collected from patients at multiple timepoints after infusion were assessed by qPCR. The green dots represent the CAR-NK copy numbers in peripheral blood samples for the nine patients who received a partially HLA-matched CAR-NK product (4/6 HLA match). The red dots represent the CAR-NK copy numbers for the two patients who received a non-HLA matched product (1/6 or 2/6 HLA match). The dotted black line represents the level of detection of the PCR assay.

FIGS. 16A-16D: Detection of CAR-NK cells by multiparameter flow cytometry FIG. 16A shows the flow cytometry gating strategy for the detection of donor CAR-NK cells in the peripheral blood in a representative patient (patient 6, day +3 after CAR-NK infusion). Lymphocytes were selected using FSC-A and SSC-A (i); next doublets were excluded using SSCW vs SSC-H (ii); live cells were identified using a Live/Dead dye (iii); hematopoietic cells within the live population were then selected by gating on CD45+ cells (iv); myeloid cells were excluded by gating on the CD33 negative and CD14 negative cells (v); NK cells were identified by gating on CD3− and CD56+ cells (vi). Within the CD3-CD56+ subset, cord blood derived NK cells were identified based on expression of the donor-specific HLA-antigen (vii). Expression of CAR on donor NK cells was further determined using an antibody directed against the CH2-CH3 domain of the human IgG hinge (109606088/Jackson Immuno Rsch) (viii). B cells were identified by expression of CD19 and/or CD20 by gating on the CD45+CD33−CD14− lymphocyte population (ix). FIG. 16B shows the CAR-NK frequencies, using the gating strategy described above for patient 6 on days 8, 14 and 21 after infusion. FIG. 16C shows the CAR NK frequencies for patient 8 on days 3, 14 and 21 after infusion. FIG. 16D shows the CAR NK frequencies for patient 10 on days 3, 7 and 14 after infusion. PBMCs: peripheral blood mononuclear cells, FSC-A: forward scatter-area, FSC-H: forward scatter-height SSC-A: side scatter-area, SSC-H: side scatter-height.

FIG. 17: Serial manual gating strategy for the detection of CAR-NK cells in the lymph node in a representative patient. Flow cytometry data to show the gating strategy for the detection of donor CAR NK cells in the lymph node for patient 6. The biopsy was performed 105 days after the CAR-NK infusion to investigate residual FDG activity in a single lymph node. The lymphocyte population was selected based on FSC-A and SSC-A (i); next doublets were excluded using SSC-W vs SSC-H (ii); live cells were identified using a Live/Dead dye (iii); hematopoietic cells within the live population were then selected by gating on CD45+ cells (iv); myeloid cells were excluded by gating on the CD33 negative and CD14 negative cells (v); NK cells were identified by gating on CD3− and CD56+ cells (vi). Within the CD3− CD56+ subset, cord blood derived NK cells were identified based on expression of the donor-specific HLA-antigen (in this case the CAR NK cells were HLAA3 positive and the recipient was HLA-A3 negative) (vii). PBMCs: peripheral blood mononuclear cells, FSC-A: forward scatter-area, FSC-H: forward; scatter-height SSC-A: side scatter-area, SSC-H: side scatter-height.

FIG. 18: CAR-NK copy numbers in peripheral blood, bone marrow and lymph node in a representative patient. The figure shows the CAR-NK copy numbers measured by qPCR at various time points in peripheral blood (green circles), bone marrow (red circles) and lymph node (black circle) for patient 8. Lymph node biopsy was performed on day 56 after the CAR-NK infusion to investigate residual FDG activity in a single lymph node. Biopsy showed a necrotic mass with calcification and no evidence of lymphoma. CAR-NK transcripts could be detected by qPCR in the lymph node (118,897.4 copies/μg) at significantly higher levels (>25 fold) compared to peripheral blood and bone marrow samples collected during the same time period.

FIG. 19: Persistence of CAR-NK cells after infusion in peripheral blood and bone marrow samples. The figure shows measurements of CAR-NK cells in peripheral blood and bone marrow samples as assessed by qPCR. Green and red dots represent peripheral blood and bone marrow samples respectively. The green (peripheral blood) and the red (bone marrow) solid lines represent the median copy number at the various time points. CAR-NK transcripts were detectable at a similar levels in peripheral blood and bone marrow.

FIG. 20: Gating strategy to detect donor CAR expressing T-cells after CAR-NK infusion. Flow cytometry gating strategy for the detection of donor T-cells and donor-derived CAR expressing T-cells in the peripheral blood in a representative patient (patient 6, day +8, panels i to viii, and day +21, panels ix and x, after CAR-NK infusion). The lymphocyte gate was selected using FSC-A and SSC-A (i); next doublets were excluded using SSC-W vs SSC-H (ii); live cells were identified using a Live/Dead dye (iii); hematopoietic cells within the live population were then selected by gating on CD45+ cells (iv); myeloid cells were excluded by gating on the CD33 negative and CD14 negative cells (v); T-cells were identified by gating on CD3+CD56-cells (vi). Within the CD3+ subset, cord blood derived T-cells were identified based on expression of the donor-specific HLA-antigen (vii). Expression of CAR on donor T-cells was further determined using an antibody directed against the CH2-CH3 domain of the human IgG hinge (109606088/Jackson Immuno Rsch). The percentage indicates the frequencies of CD3+ T cells expressing the CAR molecule (viii). The panel shows minimal contamination by CAR+ donor T-cells in PBMC samples collected from the patient on days +8 (vii and viii) and day +21 (ix and x) after CAR NK infusion. Similar results were found in two additional patients with available serial samples (data not shown). PBMCs: peripheral blood mononuclear cells, FSC-A: forward scatter-area, FSC-H: forward scatter-height SSC-A: side scatter-area, SSC-H: side scatter-height.

FIGS. 21A-21R: Levels of inflammatory cytokines in the peripheral blood. Panels show the time course for inflammatory cytokines in peripheral blood samples after CAR-NK infusion. Horizontal lines represent median values.

FIGS. 22A-22B: Patient Characteristics.

FIG. 23: Characteristics of the infused CAR-NK cell product.

FIG. 24: CAR-NK cell persistence during the follow up. CAR-NK persistence was measured in peripheral blood using qPCR.

FIG. 25: Measurement of donor-specific antibodies in patient samples at multiple time points after CAR-NK infusion. (−) indicates that the results are not available.

FIGS. 26A-26B. Antileukemic function of CB-NK cells transduced with CAR19-CD28-zeta-2A-IL15 vector. (FIG. 26A) Transduction efficiency (85%) of CB NK cells (bottom panel) compared to non-transduced NK cells (top panel). Transduction is stable. (FIG. 26B) CAR-NK cells are more efficient at killing CD19+ Raji tumors and primary CLL compared to non-transduced (NT) ex vivo expanded and activated NK cells with equal effector function against K562 cells. P<0.001 (iC9/CAR.CD19/IL15+ Raji vs NT-NKs+ Raji); P<0.001 (iC9/CAR.CD19/IL15+CLL vs NT-NKs+CLL); P=ns (iC9/CAR.CD19/IL15+K562 vs NT-NKs+K562).

FIGS. 27A-27C: In vivo homing, proliferation and antitumor activity of iC9/CAR.19/IL15-transduced CB NK cells. (FIG. 27A) C9/CAR.19/IL15-tranduced eGFP-FFLuc-labeled CB-NK cells home to sites of disease (liver, spleen, bone marrow BM]) more efficiently than CAR.19 transduced CB-NK cells or NT-NK cells. (FIG. 27B) Infusion of iC9/CAR.19/IL15-transduced CB-NK cells into NSG mice engrafted with luciferase-labeled Raji cells results in tumor eradication, as evidenced by in vivo bioluminescence imaging. Colors indicate intensity of luminescence (red, highest; blue, lowest). (FIG. 27C). The in vivo antitumor activity of a single dose of iC9/CAR.19/IL15-transduced CB NK is significantly better than that of CB-NK cells that were either not transduced or transduced with a CAR.CD19 construct lacking IL-15. P=0.001 (iC9/CAR.CD19/IL15+ Raji vs NT-NKs+ Raji); P=0.044 (iC9/CAR.CD19/IL15+ Raji vs CAR.CD19+ Raji); P=0.006 (CAR.CD19+ Raji vs NT-NKs + Raji) P=0.182 (NT-NKs+ Raji vs Raji alone).

FIGS. 28A-28B: IL-15-transduced CB-NK cells do not show signs of autonomous or dysregulated growth. (FIG. 28A) iC9/CAR.19/IL15-transduced CB NK cells stop expanding within 6 weeks of in vitro culture with no evidence of autonomous growth. (FIG. 28B) Photomicrographs of mesenteric lymph nodes show vestigial lymphoid tissue with no lymphocytes in any experimental mice, which is typical of NSG mice. Images of the spleen show rudimentary periarteriolar lymphoid tissue devoid of lymphocytes (black arrows) and is surrounded by hematopoietic tissue composed of various stages of erythroid and myeloid series cells, including megakaryocytes and hemosiderin-laden macrophages. Bone marrow contains normal hematopoietic cells and no abnormal lymphocytes. H&E stain, magnification ×200. Slides from two representative groups of NSG mice treated with iC9/CAR.19/IL15-transduced CB NK cells.

FIG. 29: IL-15 production by iC9.CAR.19.CD28.CD3ƒ.IL15-transduced CB NK cells; iC9. CAR.19.CD28.CD3ζ.IL15-transduced CB NK cells produce IL-15 in response to antigenic stimulation in vitro.

FIGS. 30A-30B: Activation of the inducible caspase-9 suicide gene eliminates iC9/CAR.19/IL15+ CB-NK cells. (FIG. 30A) The addition of 10 nM of AP1903 to cultures of iC9-CAR-IL15+ CB-NK cells induced apoptosis/necrosis of transgenic cells (bottom right panel) within 4 hours as assessed by annexin-V-7AAD staining. NT, non-transduced CB-NK cells; CAR, iC9/CAR.19/IL15-transduced NK cells; (FIG. 30B) NSG mice engrafted i.v. with Raji cells, and infused with iC9/CAR.19/IL15+ CB-NK cells were treated 10-14 days later with two doses of the AP1903 dimerizer (50 μg) i.p. two days apart. iC9/CAR.19/IL15-expressing NK cells were substantially reduced in all organs tested 3 days later.

### DESCRIPTION OF ILLUSTRATIVE EMBODIMENTS

Encouraging clinical results have been seen with human umbilical cord blood-derived natural killer (CB-NK) cells transduced with retroviral vectors targeting CD19⁺ lymphoid cancers. A number of additional CAR-NK cell constructs have been generated targeting myeloid tumors, multiple myeloma and solid tumor cancer antigens. In some embodiments, the present disclosure provides methods for the robust expansion of NK cells. The cells may be obtained from frozen or thawed CB units and expanded in gas permeable bioreactors containing co-cultures with antigen presenting cells (APCs), such as universal antigen presenting cells (uAPCs), or other feeder cells and cytokines, such as interleukin (IL)-2.

One limitation of using CAR NK cells for clinical therapy is because of their small numbers and their poor survival post thaw. The present studies have addressed both of these limitations by using GMP-compliant strategy for the ex vivo expansion of CAR NK cells. The present methods resulted in a median 2200-fold expansion in two weeks, with an excellent CAR transduction efficiency of around 66%. Using this strategy, up to 400 doses of 1×10⁶ CAR NK cells per kg can be generated for the treatment of patients.

Accordingly, certain embodiments of the present disclosure provide methods and compositions concerning the manufacture, expansion, quality control, and functional characterization of clinical-grade NK cells intended for cell and immunotherapy. Growing and molding clinically relevant numbers of NK cells for infusion into patients while meeting time constraints are extremely challenging even in the best of circumstances. The disclosed methods and compositions detail the technical processes of NK cell manufacture, details and kinetics of achievable NK cell expansions, and molecular characterization to verify successful cellular molding.

The present methods provide high and consistent transduction levels of the NK cells with the CAR constructs and rapid production of highly potent CB-CAR-NK cells which can be infused fresh or frozen for subsequent infusion. The frozen CAR-NK cell products provided herein are truly “off-the-shelf” cell therapy which can be thawed and infused into patients with no delay needed for production.

In addition, the present studies have demonstrated the safety of infusing NK cells and CAR-NK cells which are not HLA-matched with the patients. Thus, the combination of no longer needing HLA matching and the robust efficacy of thawed products has resulted in a paradigm shift in the use of CAR-based therapy where CAR-NK cells can now be prepared as an “off-the-shelf” product that can be infused as a point of care product. The present strategy can also be applied to NK cells from any source, including peripheral blood, bone marrow, hematopoietic stem cells, induced pluripotent stem cells or NK cell lines.

In specific aspects, the NK cells may be isolated from umbilical CB of healthy donors co-cultured with APCs, such as K-562-based feeders or other feeder cells such as lymphoblastoid cells lines or beads, and one or more cytokines including IL-2, IL-15, IL-12, IL21 or IL-18. The NK cells may then be transduced with retroviral, lentiviral, adenoviral, or adeno-associated viral vectors, or electroporated with sleeping beauty or piggy-back constructs that target hematologic and solid cancers. The transduced cells may then be further expanded in gas permeable bioreactors containing co-cultures with the APCs or other feeders and IL-2 or other cytokines to obtain the potent CAR-transduced CB-NK cells. Those cells can be infused fresh, or can be frozen for thaw and infusion at a later date.

CAR-T cells for infusion in the allogeneic setting must be HLA-matched or be genetically manipulated to remove the T cell receptor in order to prevent lethal GVHD. Previous studies have used CB units to generate clinical NK cell and CAR-NK cell products that were matched at 4/6 HLA antigens for safety and consistency with the requirements for CB transplant matching. However, in the present studies, 2 patients were treated with allogeneic CB-derived NK cells that were not HLA-matched at any antigen to the patients with no toxicity. They were infused safely with no GVHD or other toxicities, and comparable persistence in the patients compared to 4/6 HLA-matched NK cells. This has established the present platform for using CB units to generate NK and CAR-NK cell products without the need for any HLA matching. Choosing CB units at random from CB banks for NK cell production markedly expands the number of CB units available and improves the timing and logistics of therapy by eliminating the need for HLA typing of the patient and then matching with the CB unit.

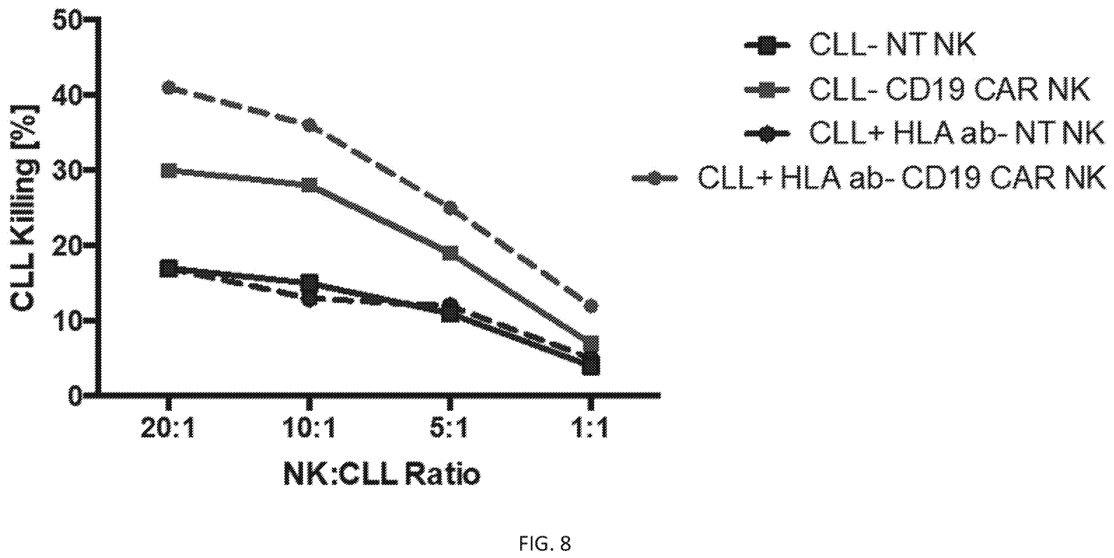

In further aspects, the present methods may comprise identifying and selecting CB units for CAR NK production which are typed for the killer immunoglobulin receptor (KIR) ligand and are mismatched with the recipient. The resulting alloreactivity from KIR-ligand mismatch may further enhance the activity of CAR-transduced NK cells by synergizing with the CAR-mediated recognition of the tumor cells. Indeed, the present studies have shown that blocking the KIR-ligand interaction using HLA blocking antibodies can significantly enhance the CAR-NK mediated cytotoxicity of CLL targets (FIG. 8).

The present CAR-transduced NK cells can provide an off-the-shelf source of cells for the immunotherapy of many cancers including both liquid and solid tumors. Retroviral transduction of CB derived NK cells allows for longer persistence and improved efficacy of the engineered cells for use in the immunotherapy of many cancers and potentially for the treatment of infections, including viruses, bacteria and fungi and autoimmune disorders by targeting autoreactive B or T cells.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “haplotyping or tissue typing” refers to a method used to identify the haplotype or tissue types of a subject, for example by determining which HLA locus (or loci) is expressed on the lymphocytes of a particular subject. The HLA genes are located in the major histocompatibility complex (MHC), a region on the short arm of chromosome 6, and are involved in cell-cell interaction, immune response, organ transplantation, development of cancer, and susceptibility to disease. There are six genetic loci important in transplantation, designated HLA-A, HLA-B, HLA-C, and HLA-DR, HLA-DP and HLA-DQ. At each locus, there can be any of several different alleles.

A widely used method for haplotyping uses the polymerase chain reaction (PCR) to compare the DNA of the subject, with known segments of the genes encoding MHC antigens. The variability of these regions of the genes determines the tissue type or haplotype of the subject. Serologic methods are also used to detect serologically defined antigens on the surfaces of cells. HLA-A, -B, and -C determinants can be measured by known serologic techniques. Briefly, lymphocytes from the subject (isolated from fresh peripheral blood) are incubated with antisera that recognize all known HLA antigens. The cells are spread in a tray with microscopic wells containing various kinds of antisera. The cells are incubated for 30 minutes, followed by an additional 60-minute complement incubation. If the lymphocytes have on their surfaces antigens recognized by the antibodies in the antiserum, the lymphocytes are lysed. A dye can be added to show changes in the permeability of the cell membrane and cell death. The pattern of cells destroyed by lysis indicates the degree of histologic incompatibility. If, for example, the lymphocytes from a person being tested for HLA-A3 are destroyed in a well containing antisera for HLA-A3, the test is positive for this antigen group.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, and dendritic cells, or molecules, naturally occurring or synthetic capable of presenting antigen, such as purified MHC Class I molecules complexed to ÿ2-microglobulin.

### II. ENGINEERED NK CELLS

In certain embodiments, the present disclosure provides methods for producing antigen receptor engineered (e.g., CAR and/or TCR) NK cells comprising incubating the cells with artificial presenting cells (APCs) and cytokines, transducing the cells with a CAR construct, and expanding the cells in the presence of APCs and cytokines. The CAR and/or TCR construct may be a retroviral or lentviral vector or may be electroporated. The method may comprise obtaining a starting population of cells from cord blood, peripheral blood, bone marrow, CD34⁺ cells, or iPSCs, particularly from cord blood. The starting cell population may then be subjected to a Ficoll-Paque density gradient to obtain mononuclear cells (MNCs). The MNCs can then be depleted of CD3, CD14, and/or CD19 cells for negative selection of NK cells or may be positively selected by CD56 selection. The NK cells may then be incubated with APCs and cytokines, such as IL-2, IL-21, and IL-18 followed by CAR transduction, such as retroviral transduction. The engineered NK cells can be further expanded in the presence of irradiated APCs and cytokines, such as IL-2.

The APCs used in the present methods may be K-562-based feeder cells, lymphoblastoid cell lines, or universal antigen presenting cells (uAPCs), or a non-cell based approach, for instance using beads, cell particles or exosomes. “UAPC(s)” refer herein to antigen presenting cells designed for the optimized expansion of immune cells, specifically NK cells. The UAPCs may be generated by a unique combination of co-stimulatory molecules to overcome inhibitory signals and induce optimal and specific NK cell killing function. Exemplary APCs are generated by enforced expression of membrane-bound interleukin 21(mbIL-21) and 4-1BB ligand in the NK cell-sensitive K562 antigen-presenting cell line (APC) (referred to as clone 46). In another embodiment, UAPCs were produced by enforced expression of mbIL-21, 4-1BB ligand, and CD48 in K562 cells (termed universal APC (UAPC)). In another embodiment, UAPCs were generated by enforced expression of mbIL-21, 4-1BB ligand, and CS1 in K562 cells (termed UAPC2). The UAPCs may be generated to express mbIL-21, 41BBL, and an NK-cell specific antigen, such as a SLAM family antigen.

The engineered and expanded NK cells of the present disclosure are less likely to cause graft-versus-host disease (GVHD) than off-the-shelf CAR T cells in the absence of full HLA-matching. In addition, the CB-derived engineered NK cells, such as CAR NK or TCR NK cells, may be used to generate banks of NK cells for immunotherapy without the need to recruit donors for NK cell collection.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, or stem cells. In particular embodiments, the immune cells are isolated from pooled CB. The CB may be pooled from 2, 3, 4, 5, 6, 7, 8, 10, or more units. The immune cells may be autologous or allogeneic. The isolated NK cells may be haplotype matched for the subject to be administered the cell therapy. NK cells can be detected by specific surface markers, such as CD16 and CD56 in humans.

In certain aspects, the starting population of NK cells is obtained by isolating mononuclear cells using ficoll density gradient centrifugation. The cell culture may be depleted of any cells expressing CD3, CD14, and/or CD19 cells and may be characterized to determine the percentage of CD56⁺/CD3⁻ cells or NK cells.

The cells are expanded in the presence of APCs, particularly irradiated APCs, such as UAPCs. The expansion may be for about 2-30 days or longer, such as 3-20 days, particularly 12-16 days, such as 12, 13, 14, 15, 16, 17, 18, or 19 days, specifically about 14 days. The NK cells and APCS may be present at a ratio of about 3:1-1:3, such as 2:1, 1:1, 1:2, specifically about 1:2. The expansion culture may further comprise cytokines to promote expansion, such as IL-2, IL-21, and/or IL-18. The cytokines may be present at a concentration of about 10-500 U/mL, such as 100-300 U/mL, particularly about 200 U/mL. The cytokines may be replenished in the expansion culture, such as every 2-3 days. The APCs may be added to the culture at least a second time, such as after CAR transduction.

Following expansion the immune cells may be immediately infused or may be stored, such as by cryopreservation. In certain aspects, the cells may be propagated for days, weeks, or months ex vivo as a bulk population within about 1, 2, 3, 4, 5 days.

In one embodiment, the starting population of cells are MNCs isolated from a single CB unit by ficoll density gradient. The cells can then be washed and depleted of the CD3, CD14 and CD19 positive cells, such as by using the CliniMACS immunomagnetic beads (Miltenyi Biotec). The unlabeled, enriched CB-NK cells can be collected, washed with CliniMACS buffer, counted, and combined with irradiated (e.g., 100 Gy) APCs, such as in a 1:2 ratio. The cell mixture (e.g, 1×10⁶ cells/mL) may be transferred to cell culture flasks containing NK Complete Medium (e.g., 90% Stem Cell Growth Medium, 10% FBS, 2 mM L-glutamine) and IL-2, such as 50-500, such as 100-300, such as 200 U/mL. The cells can be incubated at 37° C. in 5% CO₂. On Day 3, a media change may be performed by collecting the cells by centrifugation and resuspending them in NK Complete Medium (e.g., 1×10⁶ cells/mL) containing IL-2, such as 50-500, such as 100-300, such as 200 U/mL. The cells may be incubated at 37° C. in 5% CO₂. On Day 5, the number of wells needed for Retronectin transduction can be determined by the number of CB-NK cells in culture. The RetroNectin solution may be plated to wells of 24-well culture plates. The plates can be sealed and stored in a 4° C. refrigerator.

On Day 6, a 2nd NK selection as described on Day 0 can be performed prior to transduction of the CB-NK cells. The cells can be washed with CliniMACS buffer, centrifuged and resuspended in NK Complete Medium at 0.5×10⁶/mL with IL-2, such as 100-1000, particularly 600 U/mL. The RetroNectin plates can then be washed with NK complete medium and incubated at 37° C. until use. The NK complete medium in each well can be replaced with retroviral supernatant, followed by centrifugation of plates at 32° C. The retroviral supernatant may then be aspirated and replaced with fresh retroviral supernatant. The CB-NK cell suspension containing 0.5×10⁶ cells and IL-2, 600 U/mL, may be added to each well, and the plates may be centrifuged. The plates can then be incubated at 37° C. with 5% CO₂. On Day 9, the CAR transduced CB-NK cells can be removed from the transduction plates, collected by centrifugation and stimulated with irradiated (e.g, 100 Gy) aAPCs, such as in a ratio of 1:2, in NK Complete Medium with IL-2, 200 U/mL. The cell culture flasks were incubated at 37° C. with 5% CO₂. On Day 12, media change may be performed. On Day 14, the cells can be collected by centrifugation, the supernatant may be aspirated and the cells can be resuspended in fresh NK Complete Medium containing IL-2, 200 U/mL. The cell culture flasks are incubated at 37° C. with 5% CO₂. If more than 1×10⁵ CD3⁺ cells/kg are present, a magnetic immunodepletion of CD3⁺ cells may be performed using CliniCliniMACS CD3 Reagent. On Day 15, the cells are harvested and the final product is prepared for infusion or cryopreservation.

Expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

B. Bioreactor

The NK cells may be expanded in a functionally closed system, such as a bioreactor. Expansion may be performed in a gas-permeable bioreactor, such as G-Rex® cell culture device. The bioreactor may support between 1×10⁹ and 3×10⁹ total cells in an average 450 mL volume.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel).

Hollow fiber bioreactors can be used to enhance the mass transfer during culture. A Hollow fiber bioreactor is a 3D cell culturing system based on hollow fibers, which are small, semi-permeable capillary membranes arranged in parallel array with a typical molecular weight cut-off (MWCO) range of 10-30 kDa. These hollow fiber membranes are often bundled and housed within tubular polycarbonate shells to create hollow fiber bioreactor cartridges. Within the cartridges, which are also fitted with inlet and outlet ports, are two compartments: the intracapillary (IC) space within the hollow fibers, and the extracapillary (EC) space surrounding the hollow fibers.

Thus, for the present disclosure, the bioreactor may be a hollow fiber bioreactor. Hollow fiber bioreactors may have the cells embedded within the lumen of the fibers, with the medium perfusing the extra-lumenal space or, alternatively, may provide gas and medium perfusion through the hollow fibers, with the cells growing within the extralumenal space.

The hollow fibers should be suitable for the delivery of nutrients and removal of waste in the bioreactor. The hollow fibers may be any shape, for example, they may be round and tubular or in the form of concentric rings. The hollow fibers may be made up of a resorbable or non-resorbable membrane. For example, suitable components of the hollow fibers include polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, and mixtures thereof.

The bioreactor may be primed prior to seeding of the cells. The priming may comprise flushing with a buffer, such as PBS. The priming may also comprise coating the bioreactor with an extracellular matrix protein, such as fibronectin. The bioreactor may then be washed with media, such as alpha MEM.

In specific embodiments, the present methods use a G-Rex® bioreactor. The base of the G-Rex® flask is a gas permeable membrane on which cells reside. Hence, cells are in a highly oxygenated environment, allowing them to be grown to high densities. The system scales up easily and requires less frequent culture manipulations. G-Rex® flasks are compatible with standard tissue culture incubators and cellular laboratory equipment, reducing the specialized equipment and capital investment required to initiate an ACT program.

The cells may be seeded in the bioreactor at a density of about 100-1,000 cells/cm², such as about 150 cells/cm², about 200 cells/cm², about 250 cells/cm², about 300 cells/cm², such as about 350 cells/cm², such as about 400 cells/cm², such as about 450 cells/cm², such as about 500 cells/cm², such as about 550 cells/cm², such as about 600 cells/cm², such as about 650 cells/cm², such as about 700 cells/cm², such as about 750 cells/cm², such as about 800 cells/cm², such as about 850 cells/cm², such as about 900 cells/cm², such as about 950 cells/cm², or about 1000 cells/cm². Particularly, the cells may be seeded at a cell density of about 400-500 cells/cm², such as about 450 cells/cm².

The total number of cells seeded in the bioreactor may be about 1.0×10⁶ to about 1.0×10⁸ cells, such as about 1.0×10⁶ to 5.0.0×10⁶, 5.0×10⁶ to 1.0×10⁷, 1.0×10⁷ to 5.0×10⁷, 5.0×10⁷ to 1.0×10⁸ cells. In particular aspects, the total number of cells seeded in the bioreactor are about 1.0×10⁷ to about 3.0×10⁷, such as about 2.0×10⁷ cells.

The cells may be seeded in any suitable cell culture media, many of which are commercially available. Exemplary media include DMEM, RPMI, MEM, Media 199, HAMS and the like. In one embodiment, the media is alpha MEM media, particularly alpha MEM supplemented with L-glutamine. The media may be supplemented with one or more of the following: growth factors, cytokines, hormones, or B27, antibiotics, vitamins and/or small molecule drugs. Particularly, the media may be serum-free.

In some embodiments the cells may be incubated at room temperature. The incubator may be humidified and have an atmosphere that is about 5% CO₂ and about 1% 02. In some embodiments, the CO₂ concentration may range from about 1-20%, 2-10%, or 3-5%. In some embodiments, the 02 concentration may range from about 1-20%, 2-10%, or 3-5%.

C. Genetically Engineered Antigen Receptors

The NK cells of the present disclosure can be genetically engineered to express antigen receptors such as engineered TCRs and/or CARs. For example, the NK cells are modified to express a TCR having antigenic specificity for a cancer antigen. Multiple CARs and/or TCRs, such as to different antigens, may be added to the NK cells.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

Electroporation of RNA coding for the full length TCR α and β (or γ and δ) chains can be used as alternative to overcome long-term problems with autoreactivity caused by pairing of retrovirally transduced and endogenous TCR chains. Even if such alternative pairing takes place in the transient transfection strategy, the possibly generated autoreactive T cells will lose this autoreactivity after some time, because the introduced TCR α and β chain are only transiently expressed. When the introduced TCR α and β chain expression is diminished, only normal autologous T cells are left. This is not the case when full length TCR chains are introduced by stable retroviral transduction, which will never lose the introduced TCR chains, causing a constantly present autoreactivity in the patient.

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MEW) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

1. Chimeric Antigen Receptors

In some embodiments, the CAR comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR′ immune cells (Singh et al., 2008; Singh et al., 2011).

2. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed). at the N-terminus, and one constant domain (e.g., α-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and (3 chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

D. Antigen-Presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

E. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC 000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipet alonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

F. Suicide Genes

The CAR of the immune cells of the present disclosure may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. In specific embodiments, the suicide gene is a mutant TNF-alpha that is membrane bound and may be targeted by a drug or antibody.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, CD52, EGFRv3, mutant TNF-alpha (including a membrane bound TNF-alpha) or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

G. Methods of Delivery

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

a. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

(i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, met allothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

(ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth diease virus 2A) or a “2A-like” sequence (e.g., Thosea asigna virus 2A; T2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

c. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (t k) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

d. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

H. Modification of Gene Expression

In some embodiments, the immune cells of the present disclosure are modified to have altered expression of certain genes such as glucocorticoid receptor, TGFβ receptor (e.g., TGFβ-RII), and/or CISH. In one embodiment, the immune cells may be modified to express a dominant negative TGFβ receptor II (TGFβRIIDN) which can function as a cytokine sink to deplete endogenous TGFβ.

Cytokine signaling is essential for the normal function of hematopoietic cells. The SOCS family of proteins plays an important role in the negative regulation of cytokine signaling, acting as an intrinsic brake. CIS, a member of the SOCS family of proteins encoded by the CISH gene, has been identified as an important checkpoint molecule in NK cells in mice. Thus, in some embodiments, the present disclosure concerns the knockout of CISH in immune cells to improve cytotoxicity, such as in NK cells and CD8⁺ T cells. This approach may be used alone or in combination with other checkpoint inhibitors to improve anti-tumor activity.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some aspects, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

2. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed. (See, for example, Sigma-Aldrich catalog numbers CSTZFND, CSTZFN, CTi1-1KT, and PZD0020).

3. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

4. RGENs (CRISPR/Cas Systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sG1 5-TGC TGT TGA GGA GCT GGA-3 (SEQ ID NO:1) and Ex3 NR3C1 sG2 5-AGC ACA CCA GGC AGA GTT-3 (SEQ ID NO:2). Exemplary gRNA sequences for TGF-beta receptor 2 include EX3 TGFBR2 sG1 5-CGG CTG AGG AGC GGA AGA-3 (SEQ ID NO:3) and EX3 TGFBR2 sG2 5-TGG-AGG-TGA-GCA-ATC-CCC-3 (SEQ ID NO:4). The T7 promoter, target sequence, and overlap sequence may have the sequence TTAATACGACTCACTATAGG (SEQ ID NO:5)+target sequence+gttttagagctagaaatagc (SEQ ID NO:6).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq. sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### III. METHODS OF TREATMENT

In some embodiments, the present disclosure provides methods for immunotherapy comprising administering an effective amount of the NK cells of the present disclosure. In one embodiments, a medical disease or disorder is treated by transfer of an NK cell population that elicits an immune response. In certain embodiments of the present disclosure, cancer or infection is treated by transfer of an NK cell population that elicits an immune response. Provided herein are methods for treating or delaying progression of cancer in an individual comprising administering to the individual an effective amount an antigen-specific cell therapy. The present methods may be applied for the treatment of immune disorders including auto or alloimmunity, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### IV. PHARMACEUTICAL COMPOSITIONS

Also provided herein are pharmaceutical compositions and formulations comprising immune cells (e.g., T cells or NK cells) and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### V. COMBINATION THERAPIES

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VII. EXAMPLES

The following examples are included to demonstrate particular embodiments of the disclosure. It should be appreciated by those of skill in the art that the techniques disclosed in the examples which follow represent techniques discovered by the inventor to function well in the practice of the methods of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the subject matter of the disclosure.

### Example 1—CAR NK Cell Expansion

NK cells were derived from cord blood and their specificity was redirected by genetically engineering them to express tumor-specific chimeric antigen receptors (CARs) that could enhance their anti-tumor activity without increasing the risk of graft-versus-host disease (GVHD), thus providing an ‘off-the-shelf’ source of cells for therapy, such as immunotherapy of any cancer expressing the target.

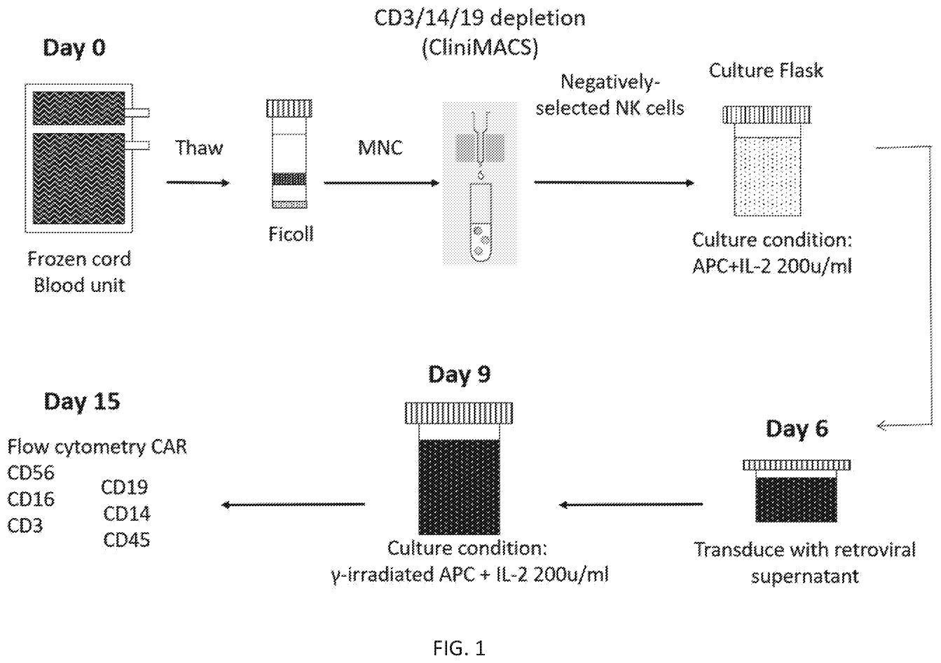

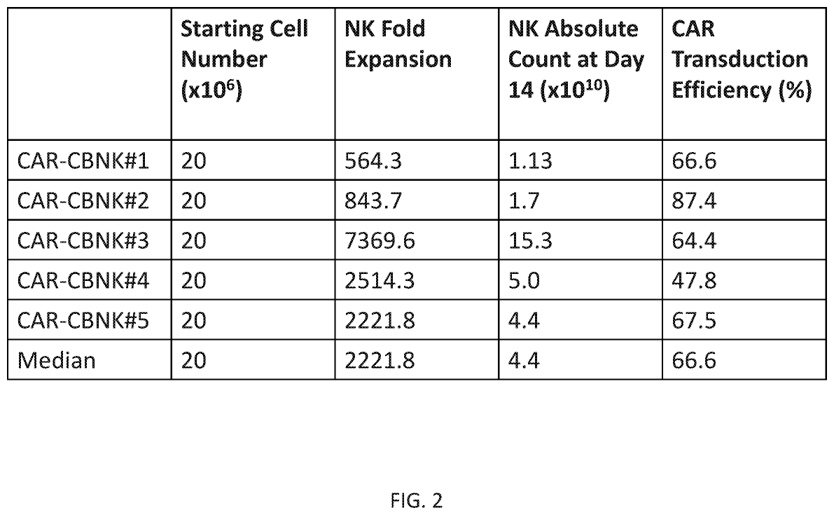

NK cells were isolated from umbilical cord blood (CB) of healthy donors and co-cultured with antigen presenting cells (APCs) and one or more cytokines including IL-2, IL-15, IL21 or IL-18. The NK cells were then transduced with a retroviral vector for CAR. The transduced cells were then further expanded in co-cultures with the APCs and IL-2 to obtain CAR-transduced CB-NK cells. These cells can be infused fresh, or can be frozen in media containing cytokines for thaw and infusion at a later date. The procedure for generating CAR CB-NK cells is summarized in FIG. 1.

Specifically, on Day 0, mononuclear cells were isolated from a single CB unit, washed and the CD3, CD14 and CD19 positive cells depleted using the CliniMACS immunomagnetic beads (Miltenyi Biotec). The unlabeled, enriched CB-NK cells were collected, washed with CliniMACS buffer, counted, and combined with irradiated (100 Gy) APCs in a 1:2 ratio (1 NK cell:2 APCs). The cell mixture (1×10⁶ cells/ml) was transferred to cell culture flasks containing NK Cell Complete Medium (NKCCM) (90% Stem Cell Growth Medium, 10% FBS, 2 mM L-glutamine) and IL-2, 200 U/mL.

The cells were incubated at 37° C. in 5% CO₂. On Day 3, a media change was performed by collecting the cells by centrifugation and resuspending them in NKCCM (1×10⁶ cells/ml) containing IL-2, 200 U/mL. The cells were then incubated at 37° C. in 5% CO₂ On Day 5, the number of wells needed for transduction was determined by the number of CB-NK cells in culture. The Retronectin solution was plated in 24-well culture plates. The plates were sealed and stored in a 4° C. refrigerator.

On Day 6, a second NK cell selection was performed as described on Day 0 prior to transduction of the CB-NK cells. The cells were washed with CliniMACS buffer, centrifuged and resuspended in NKCCM at 0.5×10⁶/ml with IL-2, 600 U/ml The Retronectin plates were then washed with NKCCM incubated at 37° C. until use. The NKCCM in each well was replaced with retroviral supernatant, followed by centrifugation of plates at 32° C. The retroviral supernatant was then aspirated and replaced with fresh retroviral supernatant. The CB-NK cell suspension containing 0.5×10⁶ cells and IL-2, 600 U/mL was added to each well, and the plates centrifuged. The plates were then incubated at at 37° C. with 5% CO₂.

On Day 9, the CAR transduced CB-NK cells were removed from the transduction plates, collected by centrifugation and stimulated with irradiated (100 Gy) aAPCs in a ratio of 1:2 (1 NK cell:2 APCs) in NKCCM with IL-2, 200 U/ml (final concentration) in the GMP-compliant G-Rex® bioreactor and incubated at 37° C. with 5% CO₂. On Day 12, IL-2 was added. On Day 15, the cells were harvested and final product prepared for infusion or cryopreservation.

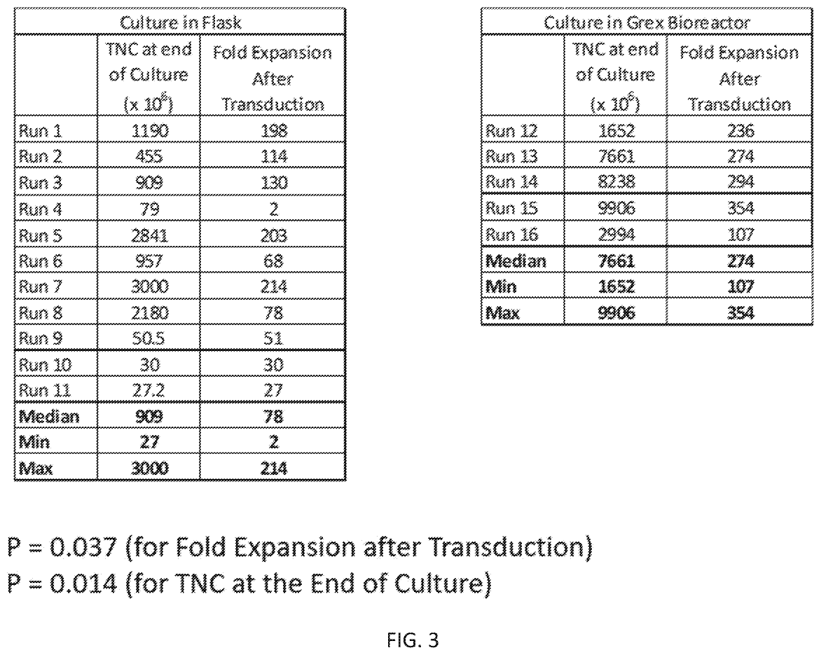

The use of a G-Rex® bioreactor following transduction of the NK cells, rather than tissue culture flasks for the entire culture period increased the robustness and reproducibility of the CAR NK cell expansions while reducing the chance of microbial contamination compared to the more open system of flasks. Additionally, it also markedly reduced the technologist time as with the culture flask system they had to manipulate the cultures every 2-3 days. With the G-Rex® the cells are fed once as described above and then left unperturbed until the Day 15 harvest. As shown in FIG. 3, from a transduced CB cell fraction containing 28 million cells a median of 7.67×10⁹ CAR NK cells were generated in the G-Rex® bioreactor compared with 0.91×10⁹ CAR NK cells in flasks (p=0.014). This represents a median 274-fold expansion following transduction in the G-Rex® bioreactor compared with a 78-fold expansion in the flasks (p=0.037) (from day 6 to day 15 of culture). With either procedure, the transduction efficiencies were excellent median around 67% (range 48-87%). Thus, the use of the G-Rex® bioreactor following the NK cell transduction step provides a excellent strategy for CAR NK cell production.

Expanded CB CAR-NK cells were frozen in GMP-compliant NK cell cryopreservation media mix with 5% DMSO, and frozen in liquid nitrogen using a rate-controlled method. In vitro chromium release assays demonstrated comparable killing of both Raji and K562 cell lines with the fresh versus frozen CAR-NK cells. In vivo killing assays using a xenogeneic NSG mouse model also confirmed comparable anti-tumor activity of frozen versus fresh NK cells against Raji tumor as assessed using bioluminescence imaging of luciferase labelled Raji cells.

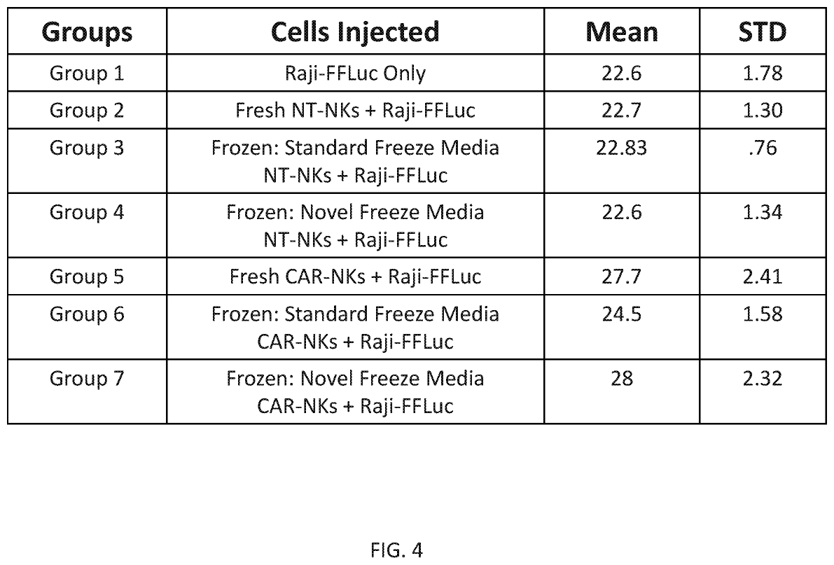

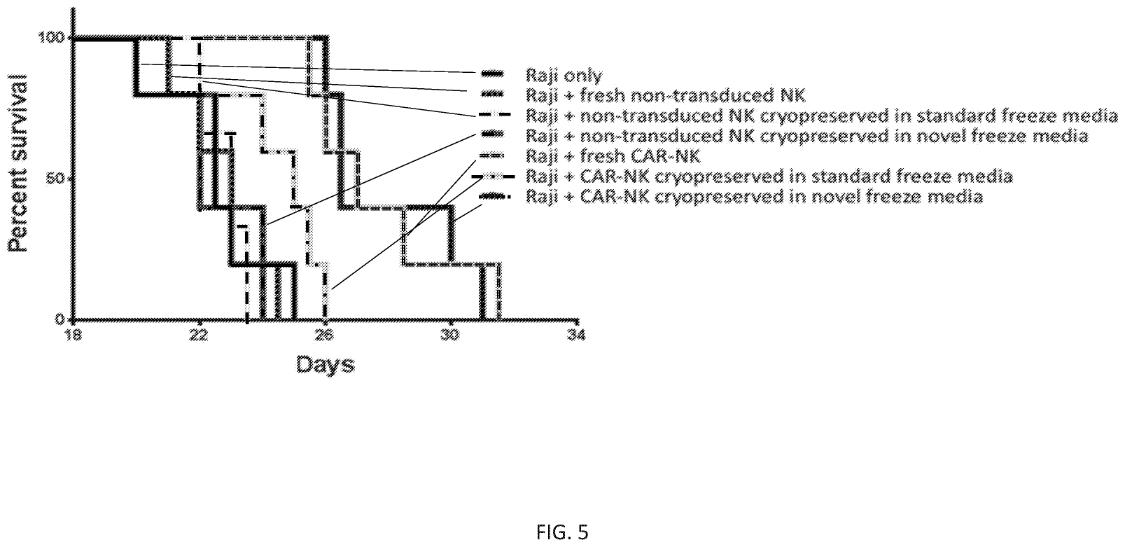

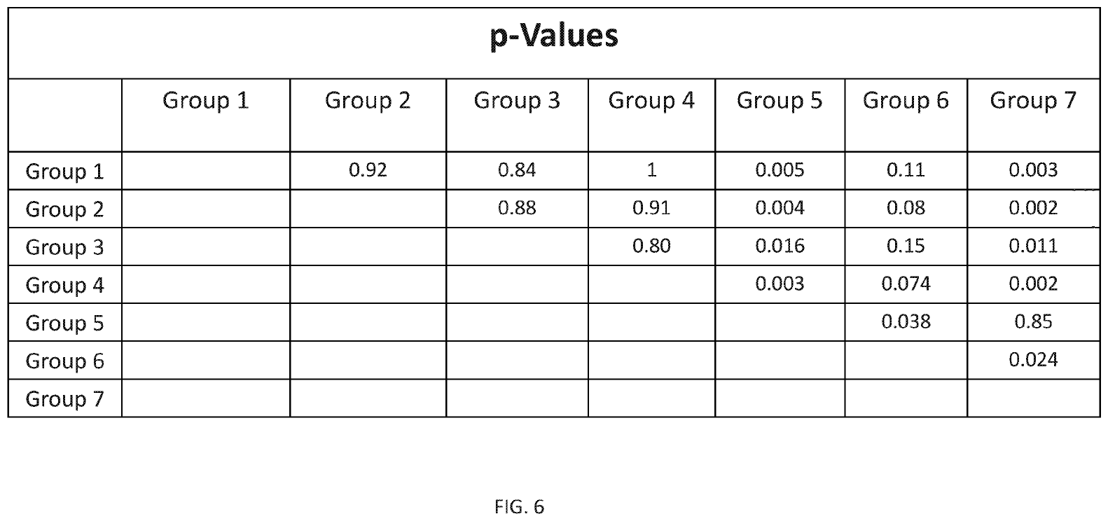

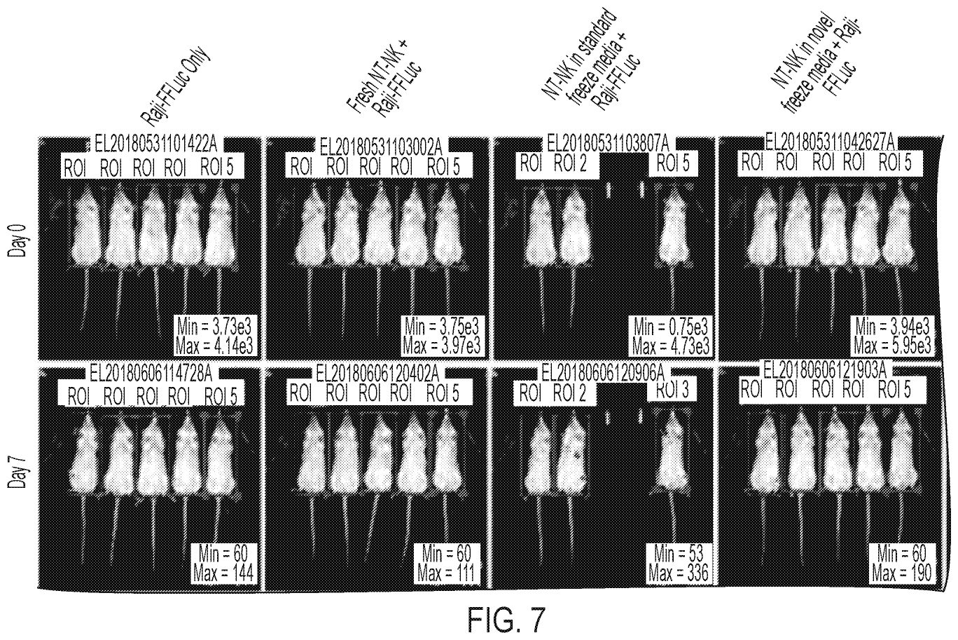

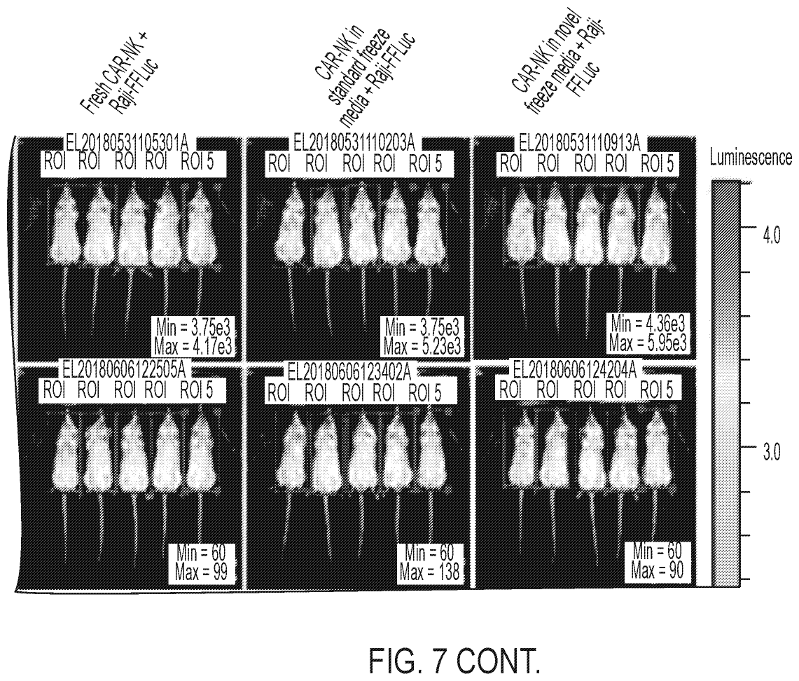

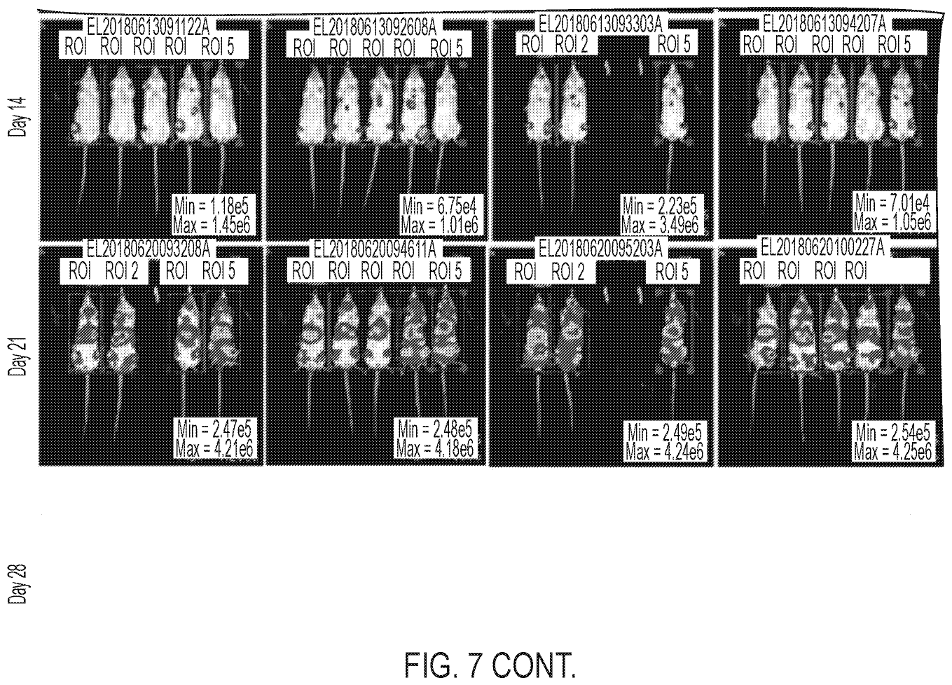

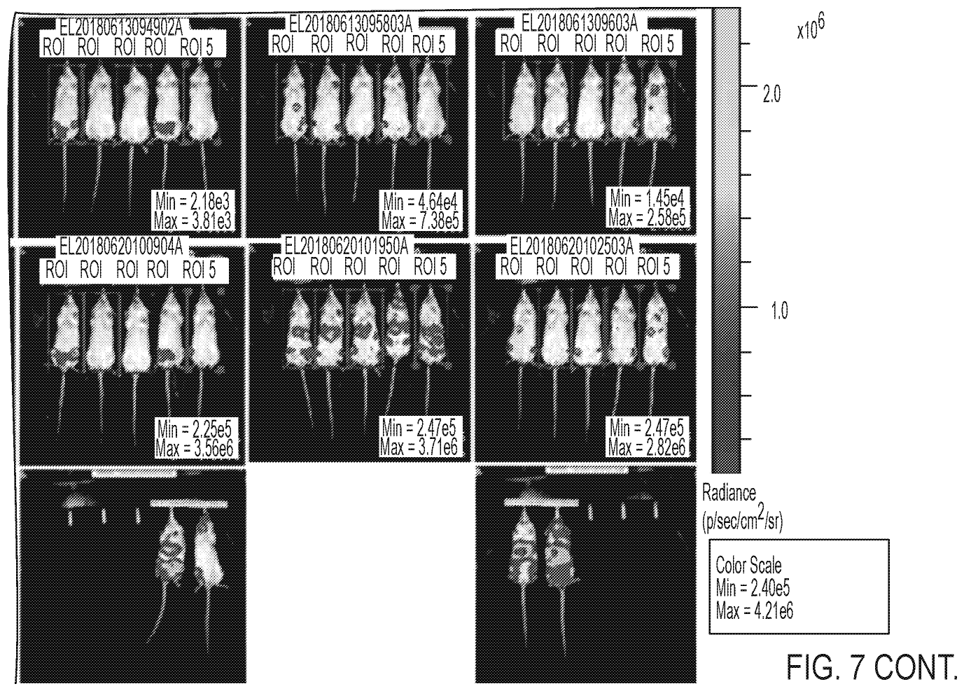

FIG. 4 shows the survival of the 7 different treatment arms and the relevant controls that were used in the in vivo NSG studies. Mice engrafted with Raji tumor and treated with the frozen CAR-NK cells had survival which was comparable to animals receiving fresh CAR-NK cells. FIG. 5 shows the survival curves for these animals and FIG. 6 shows details of the statistical analysis. FIG. 7 shows the bioluminescence imaging data showing the most potent anti-tumor activity in Raj i-bearing mice treated with either fresh CAR-NK cells or CAR-NK cells frozen with our novel cryopreservation media mix.

Using this strategy, more than 100 doses of 1×10⁶ CAR NK cells/Kg can be generated from each cord blood unit for the treatment of patients. Thus, the CAR-transduced cord blood derived NK cells can provide an off-the-shelf source of NK cells that can recognize and attack many cancers including both liquid and solid tumors. Retroviral transduction of cord blood derived natural killer cells allows for longer persistence and improved efficacy of the engineered cells for use in the immunotherapy of many cancers and potentially for the treatment of many viral infections.

### Example 2—Use of CAR-Transduced Natural Killer Cells in CD19-Positive Lymphoid Tumors

The present example concerns results of a phase 1 and 2 trial, in which HLA-mismatched anti-CD19 CAR-NK cells derived from cord blood were administered to 11 patients with relapsed or refractory CD19-positive cancers (non-Hodgkin's lymphoma or chronic lymphocytic leukemia [CLL]). NK cells were transduced with a retroviral vector expressing genes that encode anti-CD19 CAR, interleukin-15, and inducible caspase 9 as a safety switch. The cells were expanded ex vivo and administered in a single infusion at one of three doses (1×10⁵, 1×10⁶, or 1×10⁷ CAR-NK cells per kilogram of body weight) after lymphodepleting chemotherapy. As described herein, the administration of CAR-NK cells was not associated with the development of cytokine release syndrome, neurotoxicity, or graft-versus-host disease, and there was no increase in the levels of inflammatory cytokines, including interleukin-6, over baseline. The maximum tolerated dose was not reached. Of the 11 patients who were treated, 8 (73%) had a response; of these patients, 7 (4 with lymphoma and 3 with CLL) had a complete remission, and 1 had remission of the Richter's transformation component but had persistent CLL. Responses were rapid and seen within 30 days after infusion at all dose levels. The infused CAR-NK cells expanded and persisted at low levels for at least 12 months.

Study Design and Patients

The present example provides information on the first 11 patients in this study, with a data cutoff of April 2019. Briefly, patients underwent lymphodepleting chemotherapy with fludarabine (at a dose of 30 mg per square meter of body-surface area) and cyclophosphamide (at a dose of 300 mg per square meter) daily for 3 consecutive days, followed by a single infusion of the trial CAR-NK cells at escalating doses of 1×10⁵ cells, 1×10⁶ cells, and 1×10⁷ cells per kilogram of body weight. Postremission therapy was permitted after the day 30 assessment at the treating physician's discretion.

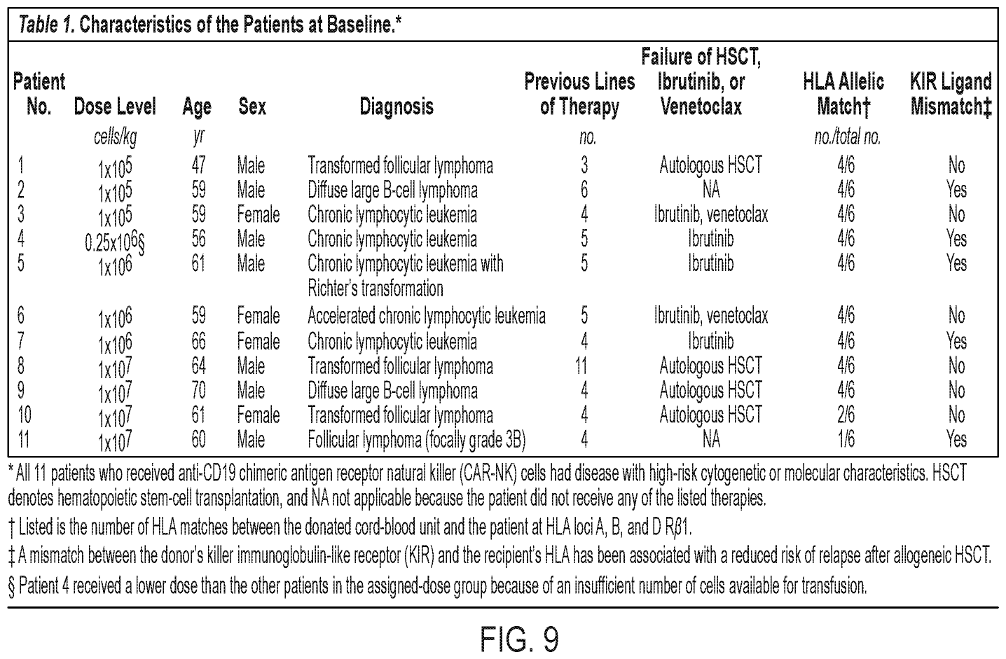

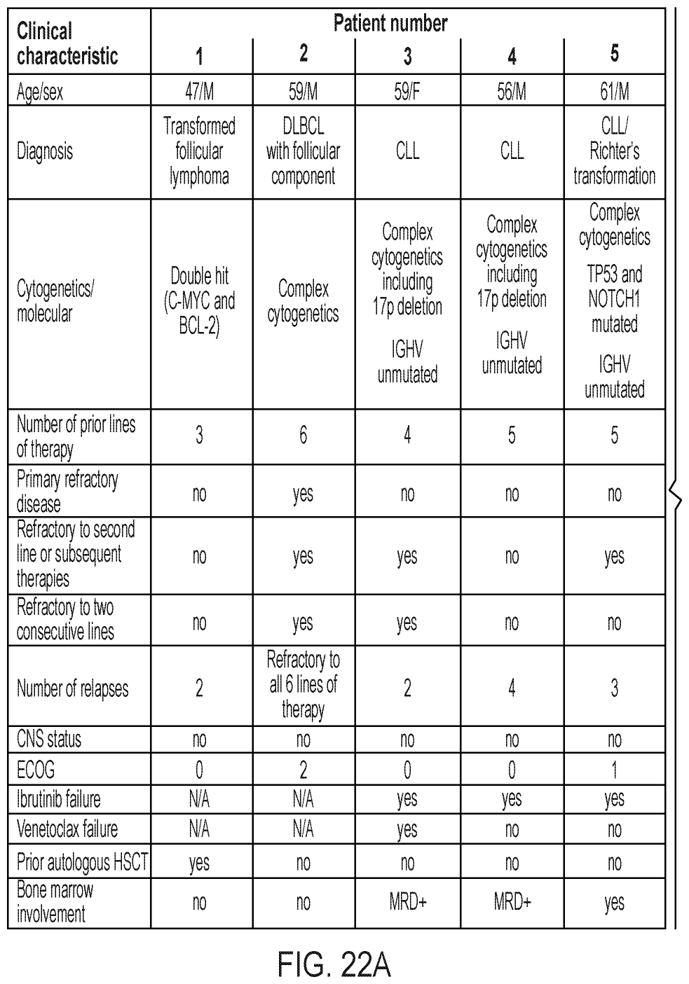

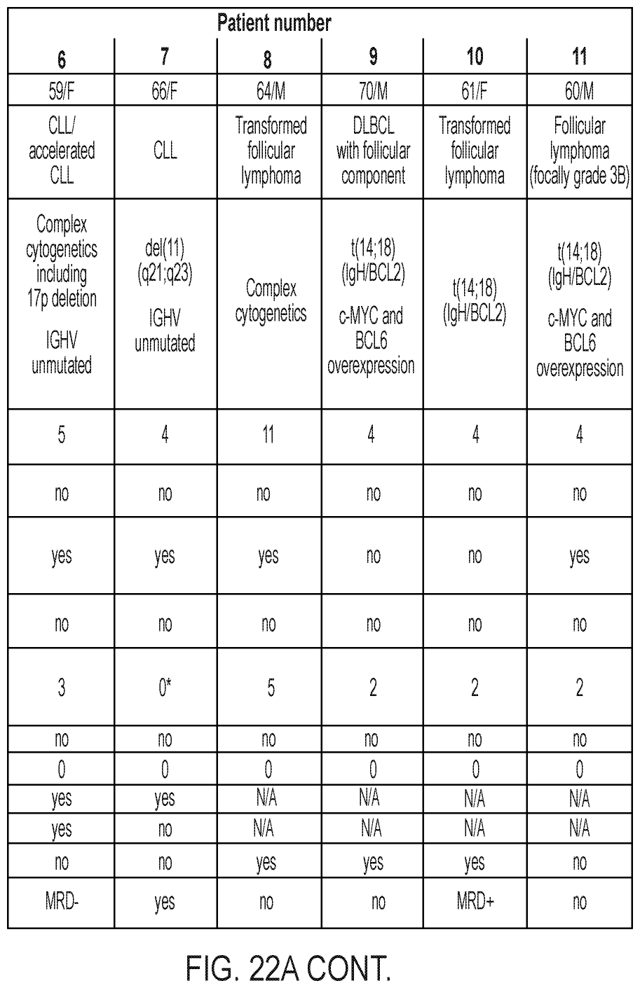

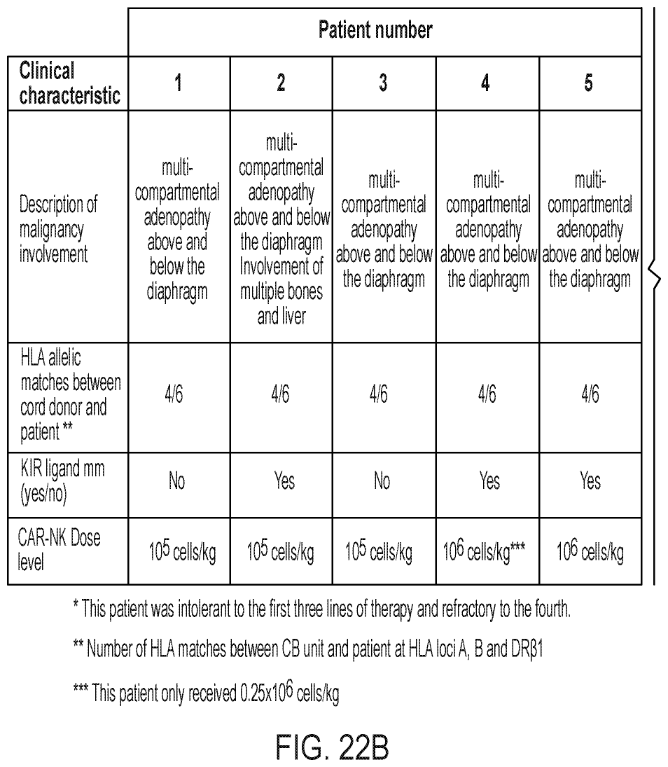

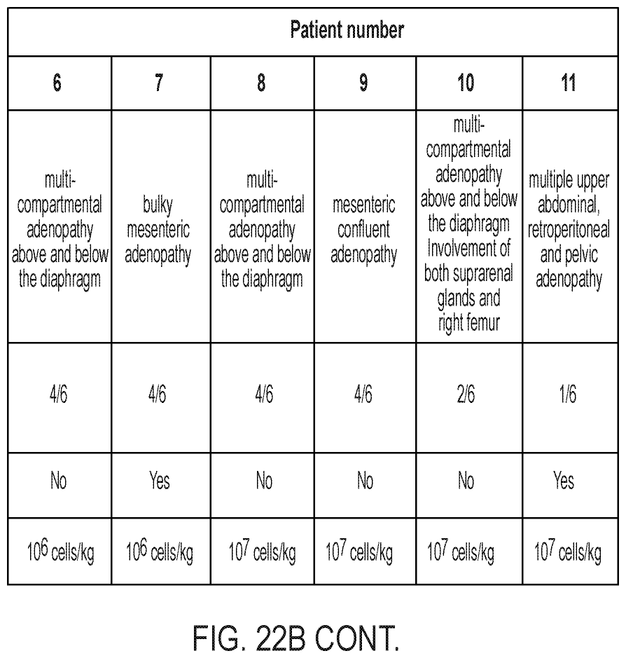

The first 9 patients received a CAR-NK product that was partially matched with the HLA genotype of the recipient (4 of 6 matches at HLA loci A, B, and DRβ1) (FIG. 9 and FIGS. 22A and 22B). The protocol was then amended to permit treatment with no consideration for HLA matching, which was the procedure used in Patients 10 and 11. When possible, a cord-blood unit was selected with killer immunoglobulin-like receptor (KIR) ligand mismatch (Mehta and Rezvani, 2016) for CAR-NK production. (KIR mismatch between the donor and recipient may enhance the intrinsic [non-CAR-mediated] antitumor activity of NK cells through a process known as missing-self recognition.) Clinical responses to therapy were based on the 2018 criteria of the International Workshop on Chronic Lymphocytic Leukemia (Hallek et al., 2018) and on the 2014 Lugano classification for non-Hodgkin's lymphoma (Cheson et al., 2014). (Further details are provided in Example 3.)

Manufacture of CAR-NK Cells from Cord Blood

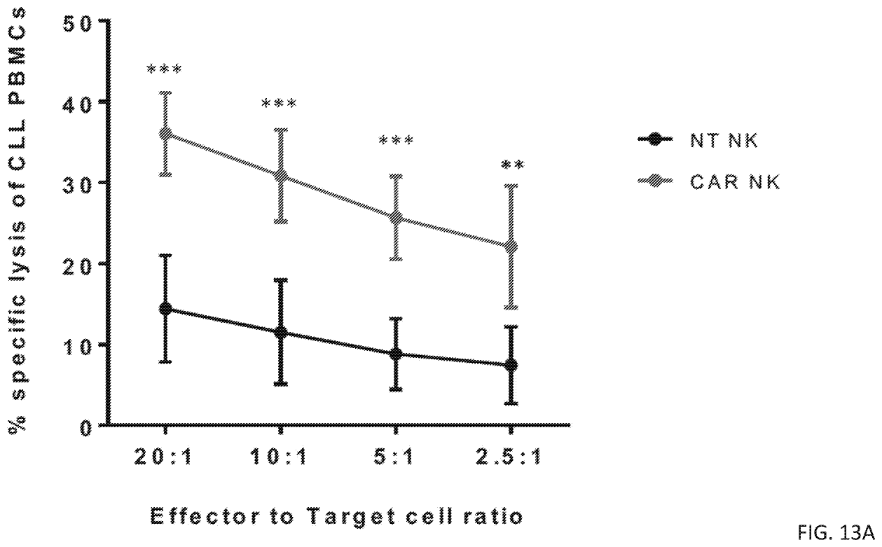

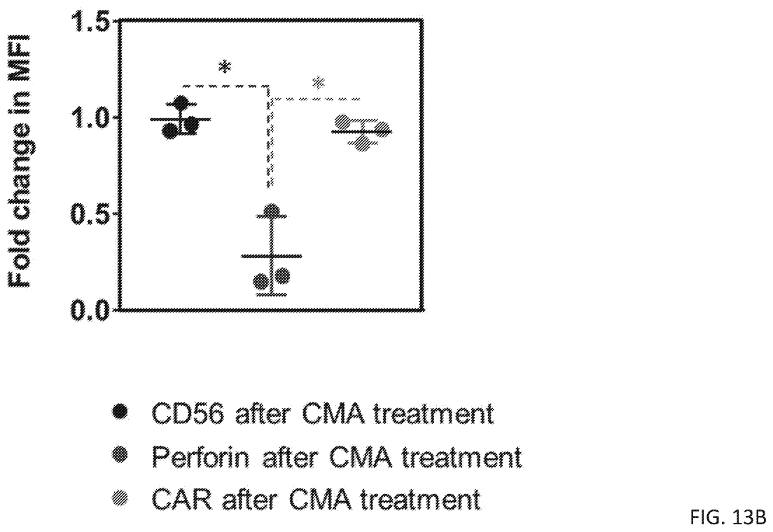

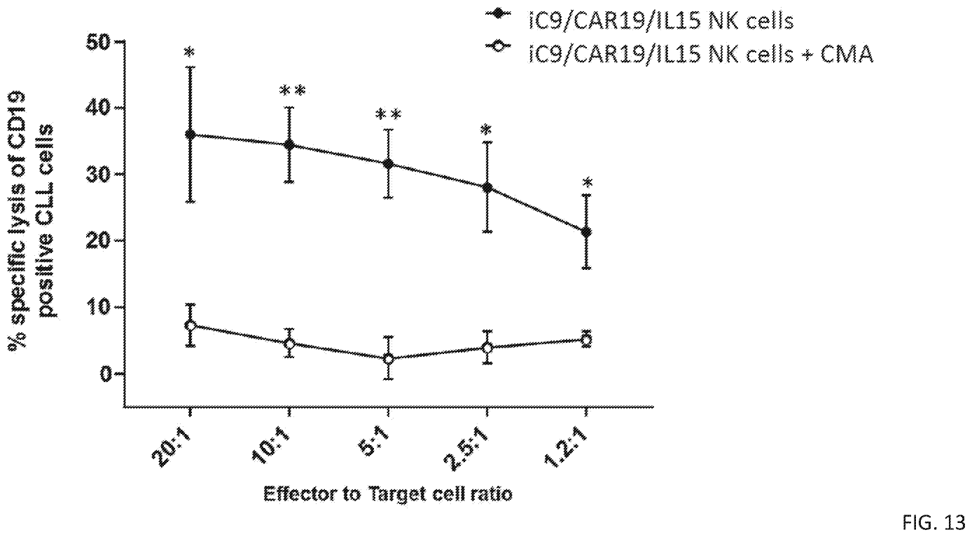

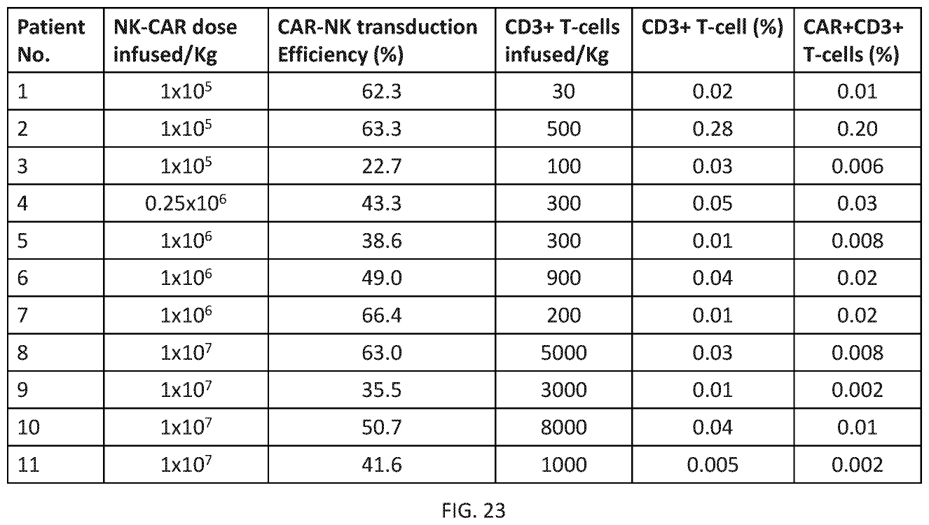

Full details regarding the manufacture of the CAR-NK cells are provided in the Methods section in Example 3. Briefly, the cord-blood unit was thawed and NK cells were purified and cultured in the presence of engineered K562 feeder cells and interleukin-2. On day 6, cells were transduced with a retroviral vector encoding the genes for anti-CD19 CAR, the CD28.CD3t signaling endodomain, interleukin-15, and inducible caspase 9 (Hoyos et al., 2010). The cells were expanded and harvested for fresh infusion on day 15. The efficiency of the final CAR-NK transduction for the infused product was 49.0% (range, 22.7 to 66.5). CAR-NK cells were tested in vitro and killed primary CLL targets in a perforin-dependent manner (FIG. 13). The median CD3-positive T-cell content in the infused product was 500 cells per kilogram (range, 30 to 8000), with a median of 0.01% (range, 0.01 to 0.002) contaminating CAR T cells in the product (FIG. 23).

Statistical Analysis

The Wilcoxon rank-sum test was used to test the associations between the response to therapy and level of CAR-NK cells. A P value of less than 0.05 was considered to indicate statistical significance.

Characteristics of the Patients

From June 2017 through February 2019, 15 consecutive patients were enrolled in accordance with the protocol. Of these patients, 4 withdrew before the initiation of treatment owing to disease progression, the development of graft-versus-host disease, the absence of detectable disease, and bacterial contamination of the product (in 1 patient each). Thus, 11 patients received a single dose of CAR-NK cells (FIG. 9 and FIGS. 22A and 22B). The median age of the patients was 60 years (range, 47 to 70). The 11 patients had already received a median of 4 lines of therapy (range, 3 to 11). Five patients had CLL (including 2 who had Richter's transformation or accelerated CLL), and all had a history of disease progression while receiving ibrutinib plus a minimum of 3 other lines of therapy; all 5 patients had high-risk genetic characteristics. Six patients had lymphoma, including 2 with diffuse large B-cell lymphoma and 4 with the follicular form; 3 of these patients underwent transformation to high-grade lymphoma. Of the 6 patients with lymphoma, 4 had undergone disease progression after autologous hematopoietic stem-cell transplantation and 2 had refractory disease.

Safety

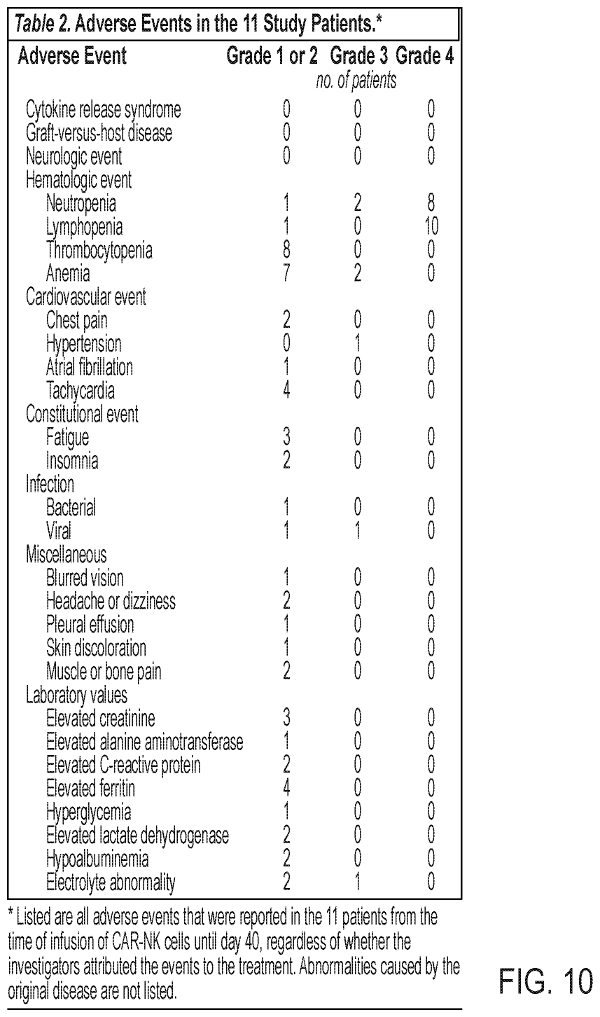

After the infusion of CAR-NK cells, none of the patients had symptoms of cytokine release syndrome, neurotoxicity, or hemophagocytic lymphohistiocytosis. Moreover, there was not observed any cases of graft-versus-host-disease, despite the HLA mismatch between the patients and their CAR-NK products. As expected, all of the patients had transient and reversible hematologic toxic events, which were mainly associated with the lymphodepleting chemotherapy. It cannot be determined whether the infusion of CAR-NK cells contributed to the hematologic toxicity. There were no cases of tumor lysis syndrome or grade 3 or 4 nonhematologic toxicity. The maximum tolerated dose of CAR-NK cells was not reached. Table 2 as FIG. 10 lists all the adverse events that were observed in the study. No patient was admitted to an intensive care unit (ICU) for management of adverse events associated with CAR-NK cells. However, Patient 2 was admitted to the ICU for treatment of progressive lymphoma and subsequently died. Given the absence of serious toxicity in the study, the inventors did not activate the caspase 9 safety switch (with rimiducid) to eliminate the CAR-NK cells.

Treatment Response

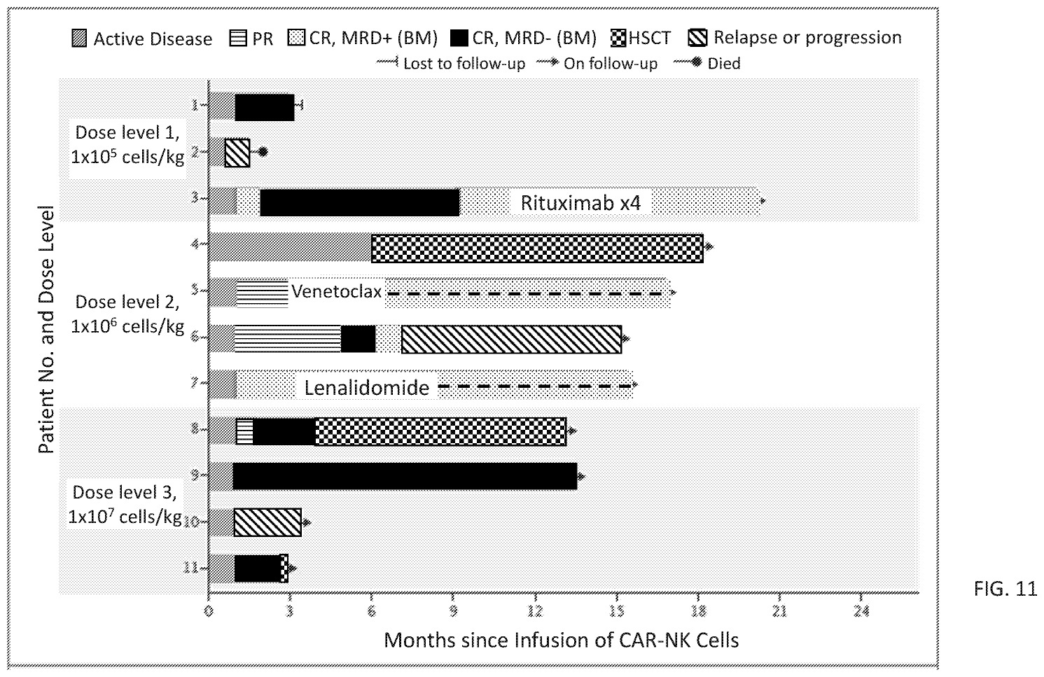

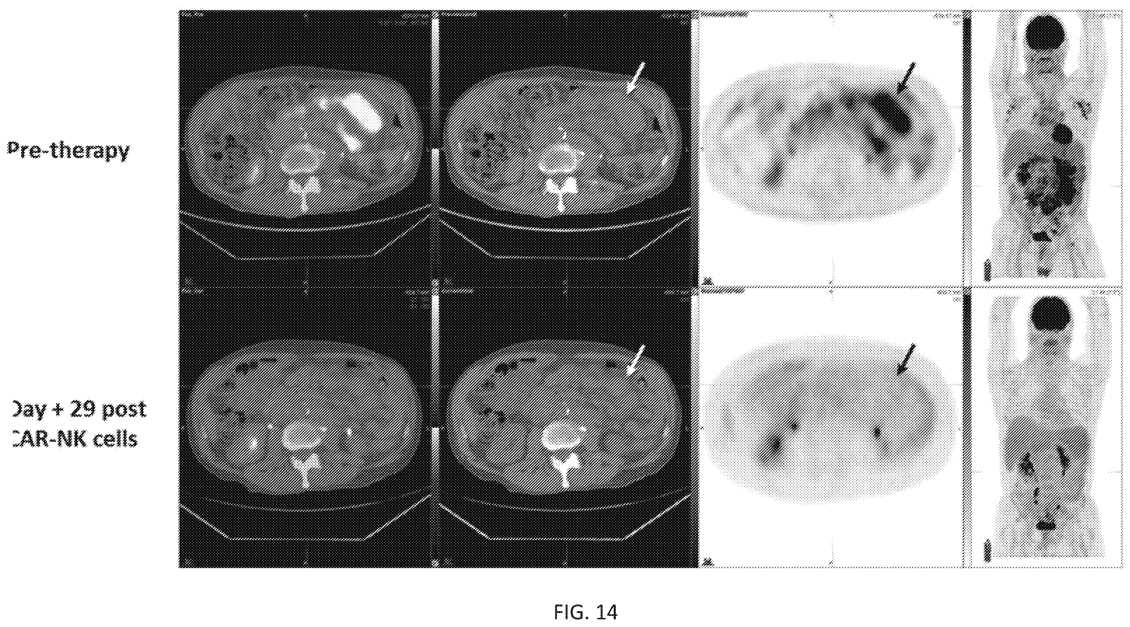

At a median follow-up of 13.8 months (range 2.8 to 20.0), 8 patients (73%) had an objective response, including 7 patients (3 with CLL and 4 with lymphoma) who had a complete response (FIG. 11). An additional patient who had CLL with Richter's transformation (Patient 5) had a complete remission of high-grade lymphoma, according to the absence of lesions with fluorodeoxyglucose uptake on positron-emission tomography-computed tomography (PET-CT) performed 30 days after the CAR-NK infusion, but continued to have cytopenia, with bone marrow infiltration by CLL (FIG. 14). Although this patient eventually had a complete response while receiving postremission therapy (see below), the invenotors did not attribute this response to the CAR-NK therapy. In all 8 patients, the response to treatment occurred during the first month after infusion. Of the 11 patients who were treated, 5 received a KIR ligand-mismatched product.

Postremission Therapy

Of the 8 patients who had a response to CAR-NK therapy, 5 underwent postremission therapy (FIG. 11). Patient 3 (who had CLL) had subsequent minimal residual disease, as detected on flow cytometry of peripheral blood, 9 months after infusion and received rituximab. Patient 7 (who also had CLL) had a clinical complete response but had persistent minimal residual disease and received lenalidomide as an immunomodulatory agent, beginning 6 weeks after infusion. Patient 8 (who had transformed follicular lymphoma) and Patient 11 (who had follicular lymphoma) underwent hematopoietic stem-cell transplantation after CAR-NK therapy while in complete response without evidence of minimal residual disease. Patient 5 (who had CLL with Richter's transformation) had remission of high-grade lymphoma but had persistent CLL and received venetoclax. All of these patients were alive and in complete remission on the date of the last assessment, although Patients 3, 5, and 7 continue to have positive results for minimal residual disease.

B-Cell Aplasia

Because B-cell aplasia has been used as a surrogate for anti-CD19 CAR T-cell activity, the frequencies were measured of CD19-positive B cells in the peripheral blood of patients after the infusion of CAR-NK cells. All of the patients except for Patients 1 and 5 had B-cell aplasia associated with previous B-cell-depleting therapies at the time of enrollment. In Patient 1, B-cell aplasia developed after CAR-NK therapy and lymphodepleting chemotherapy. Patient 5 had persistent CLL in peripheral blood, despite having had a complete response with respect to the high-grade transformation, until he received venetoclax. Patient 3 had evidence of B-cell recovery coincident with recurrent positivity for minimal residual disease. None of the remaining patients had recovery of a normal B-cell count during the follow-up period.

CAR-NK Expansion, Migration, and Persistence

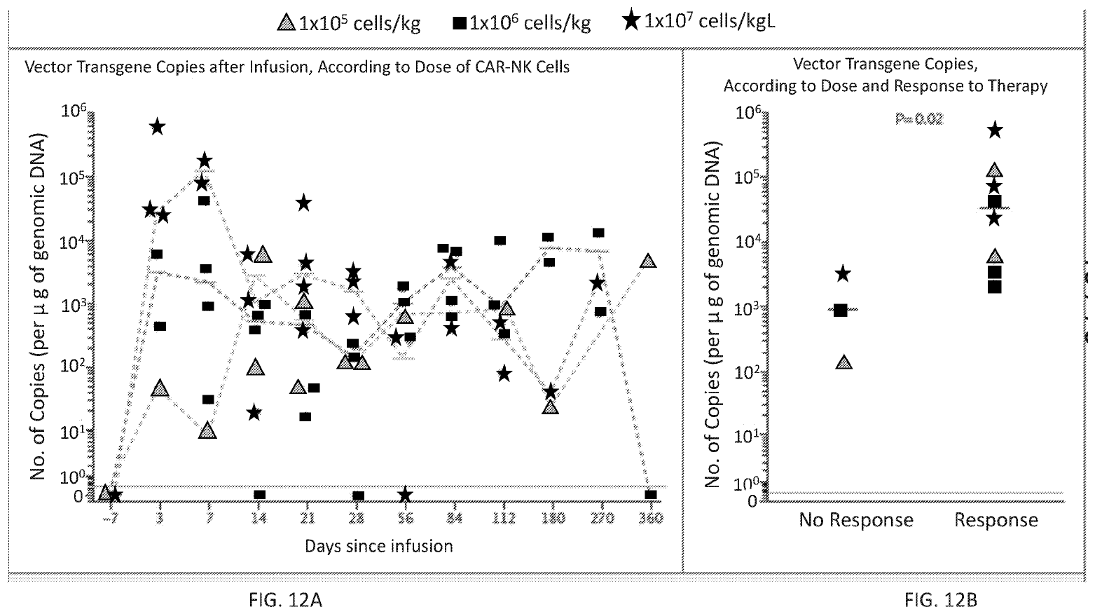

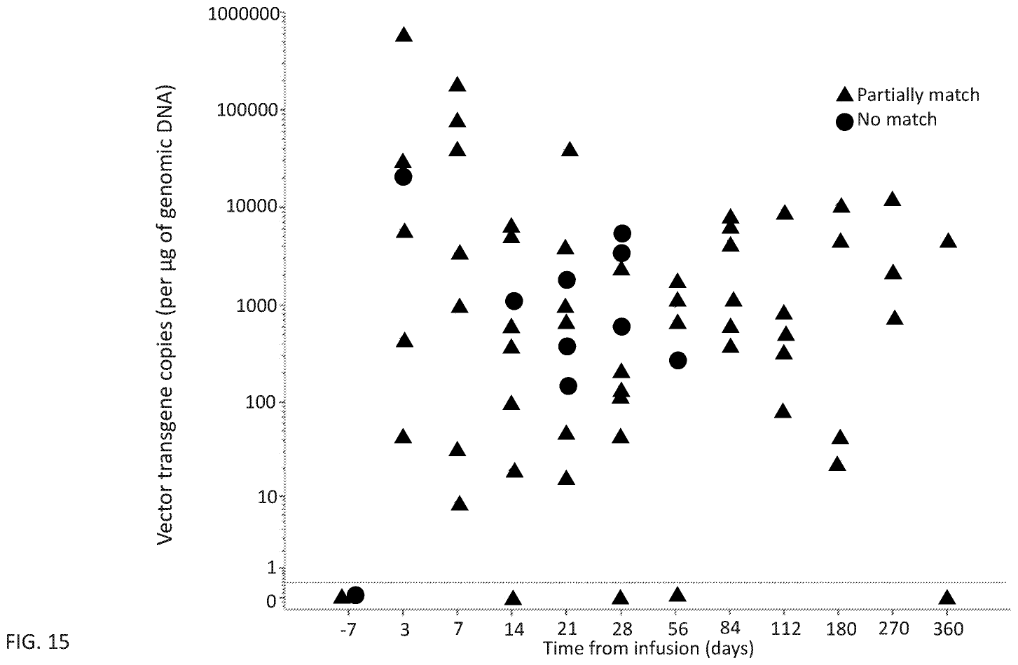

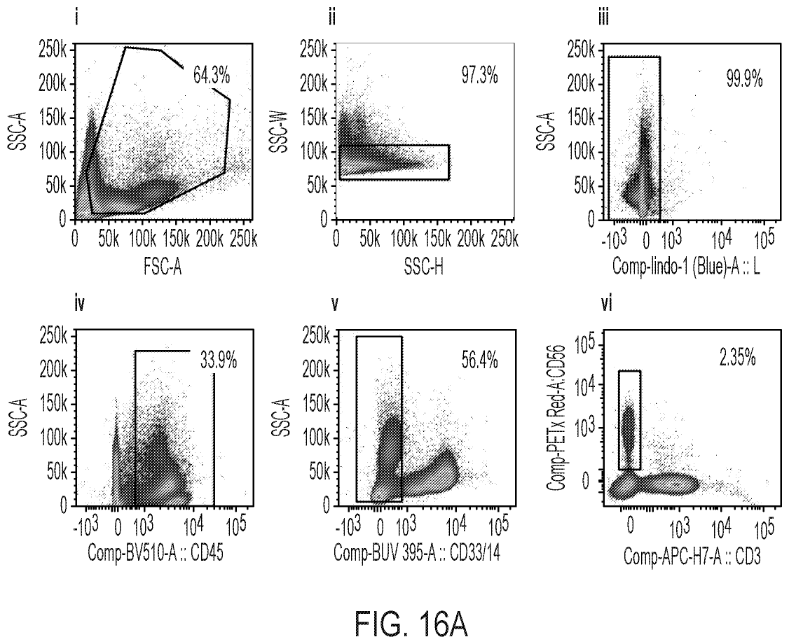

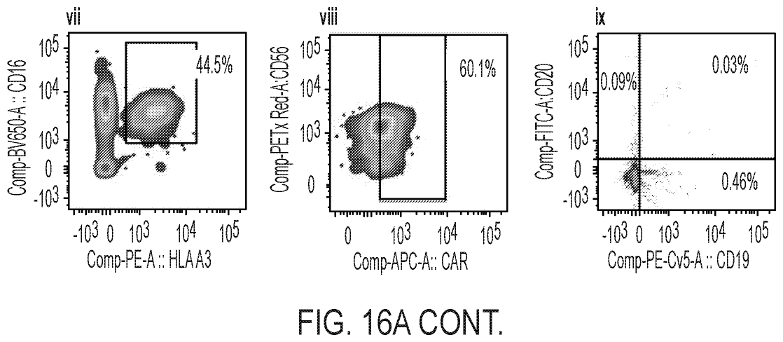

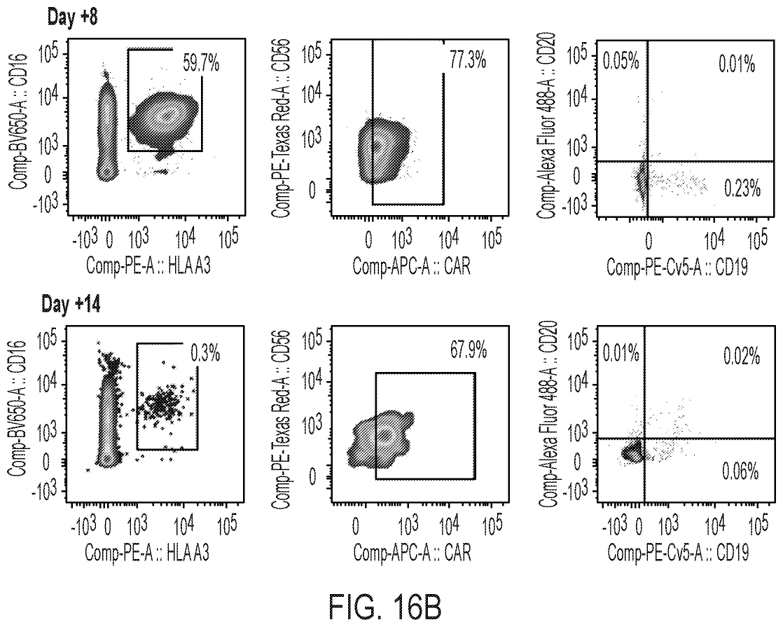

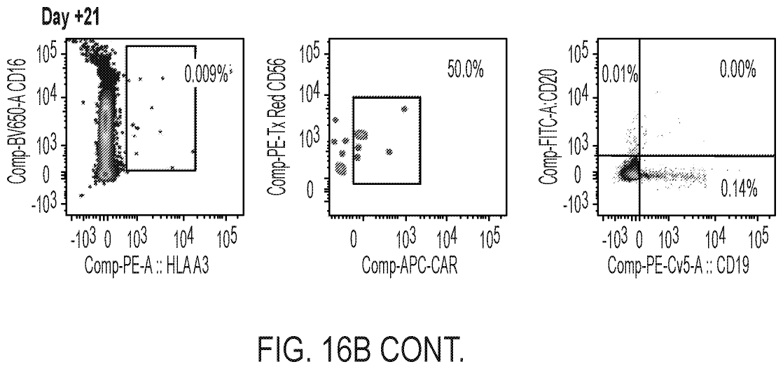

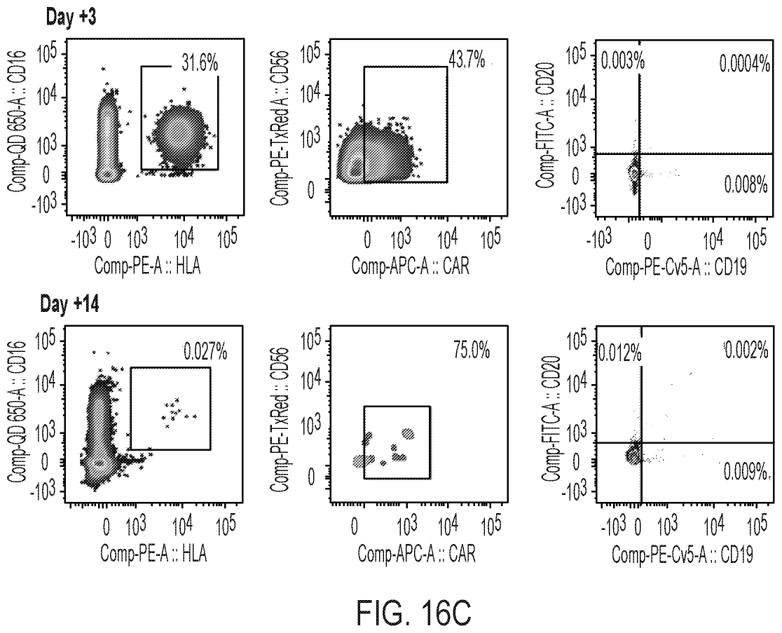

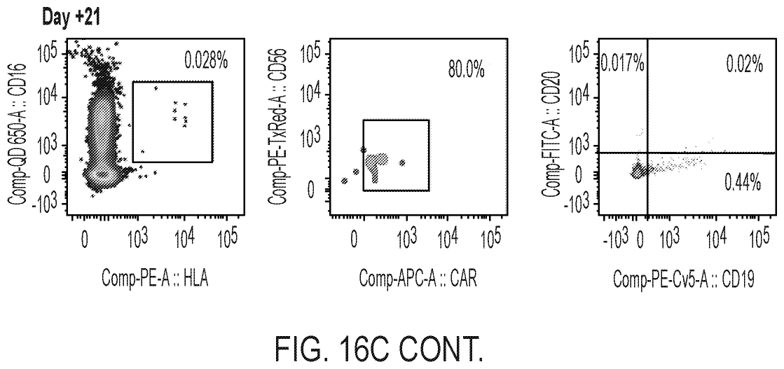

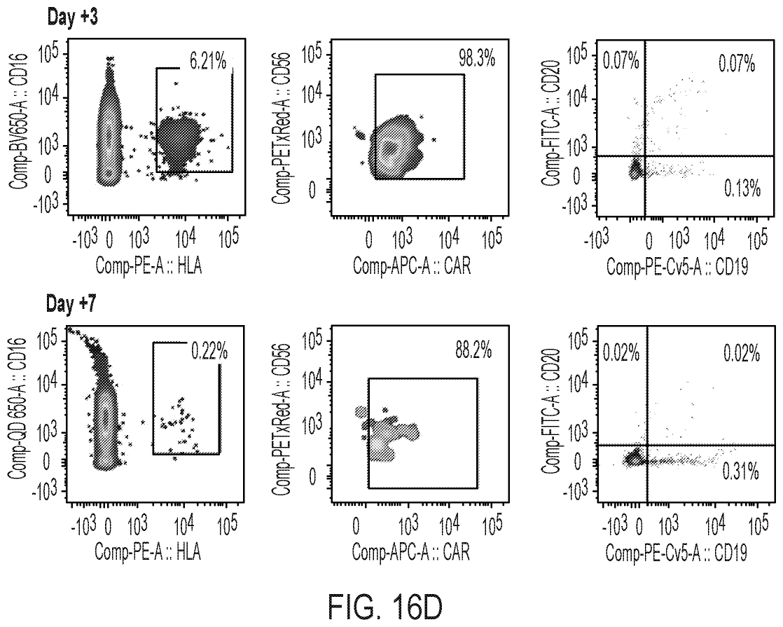

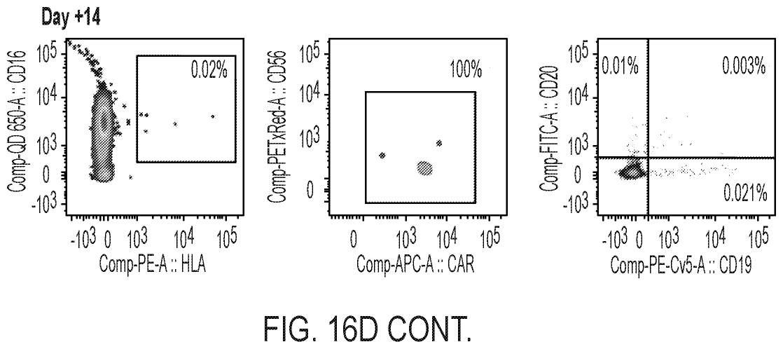

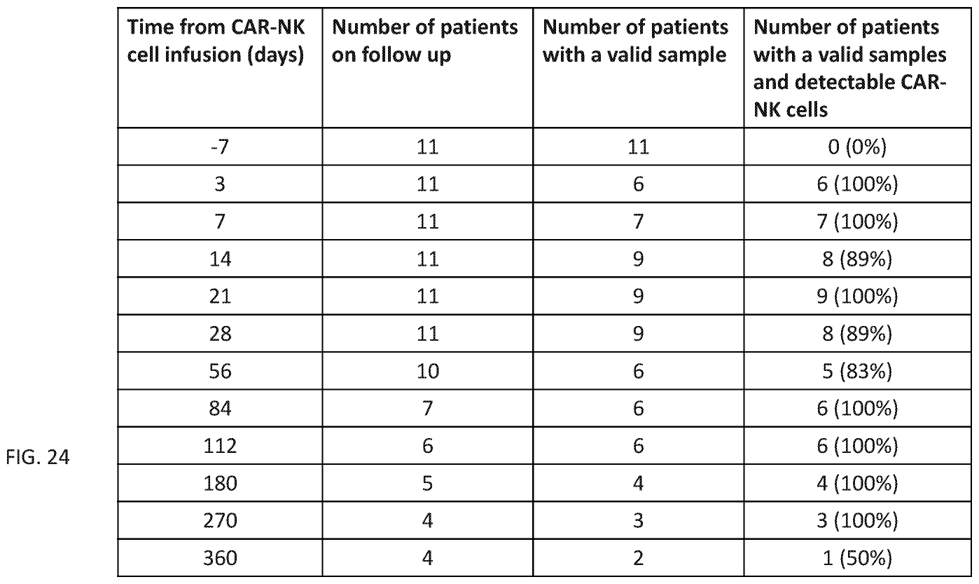

A quantitative real-time polymerase chain-reaction assay was used to measure in vivo expansion of CAR-NK cells according to the number of vector transgene copies per microgram of genomic DNA. Expansion was seen as early as 3 days after infusion, with CAR-NK cells persisting for at least 12 months (FIG. 12A and FIG. 24). The peak CAR-NK copy number was measured 3 to 14 days after infusion and was dose-dependent. Beyond day 14, no dose-related differences were noted in the level of peripheral-blood transcripts or in the persistence of CAR-NK cells. As has been reported in patients treated with CAR-T cells (Turtle et al., 2017; Neelapu et al., 2017; Maude et al., 2014) patients in our study who had a response to therapy had a significantly higher early expansion of CAR-NK cells than those who did not have a response (FIG. 12B). A difference was not observed in the persistence of CAR-NK cells according to the degree of HLA mismatch with the recipient (Table 1 in FIG. 9 and FIG. 15). These results were confirmed by means of flow cytometry (FIG. 16) (Muftuoglu et al., 2018).

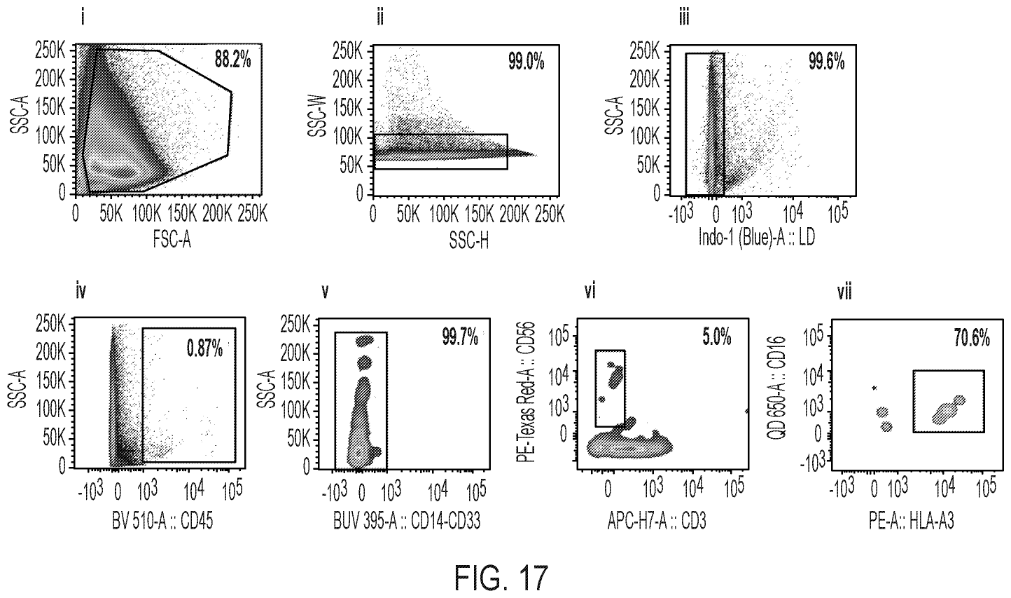

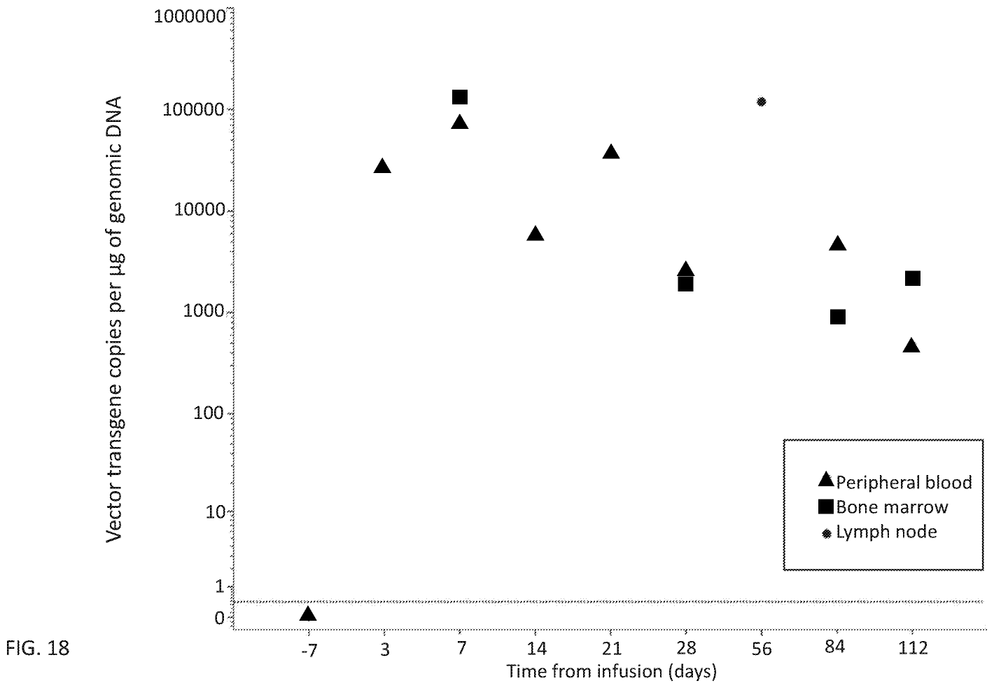

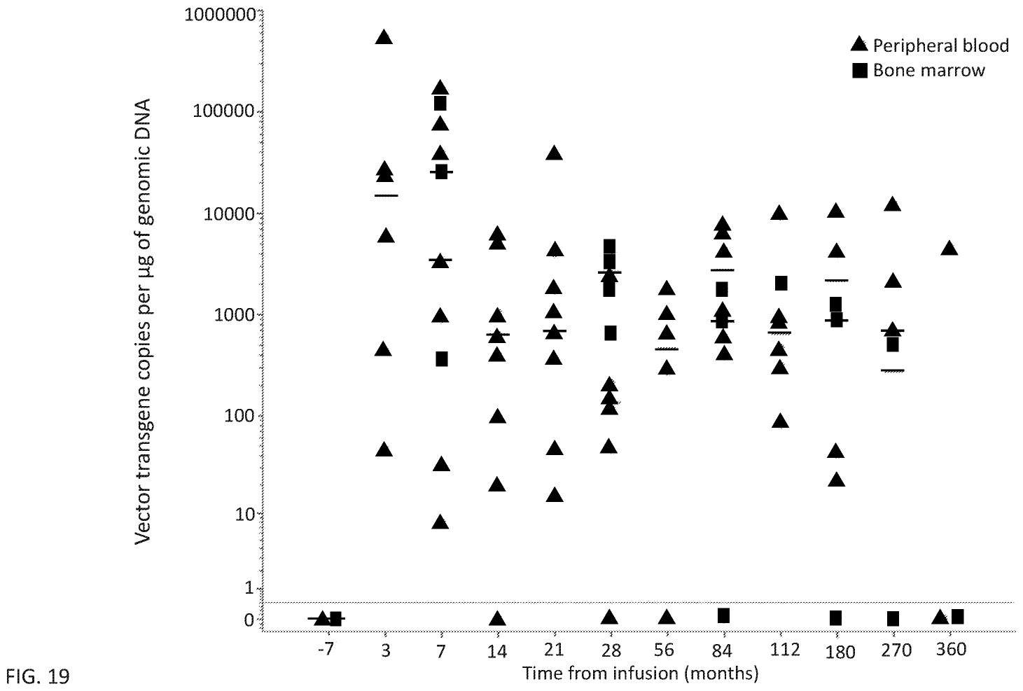

In 2 patients with available lymph-node samples, more CAR-NK cells were found in the lymph nodes than in the bone marrow or peripheral blood (FIGS. 17 and 18), a finding that supports the notion that CAR-NK cells home in on disease sites. Similar levels of CAR-NK cells were detected in the bone marrow and peripheral blood in the 10 patients with available samples (FIG. 19).

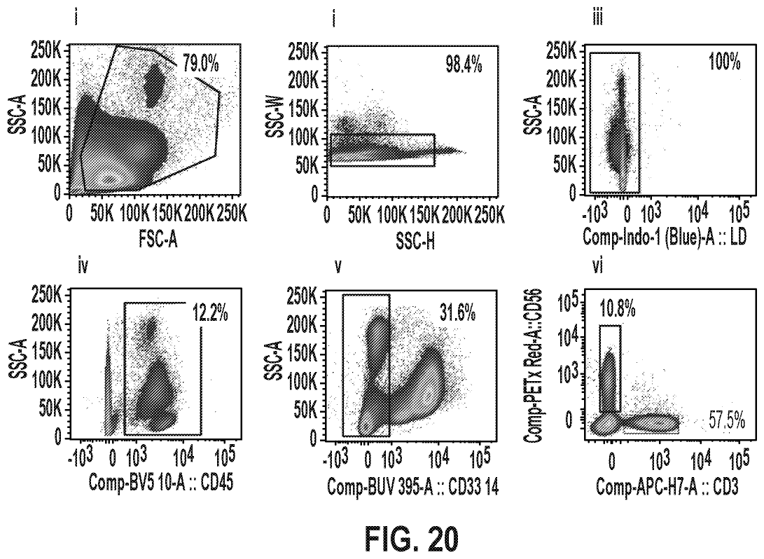

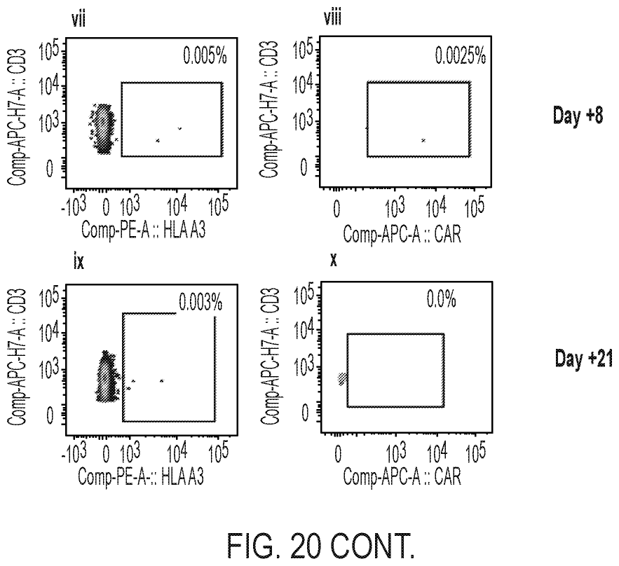

The minimal number of contaminating CAR-expressing T cells in the product did not result in detectable CAR T-cell expansion after infusion, nor did the CD3+ T cells result in the development of graft-versus-host disease (FIG. 20). CAR-NK cells were still detectable at low levels in patients who did not have a response or who had a relapse, despite the expression of CD19 in the tumor cells, which indicates in certain embodiments the presence of alternative immune escape mechanisms, such as induction of CAR-NK exhaustion. Functional studies of the residual CAR-NK cells in the patients with relapse have not been performed. The persistent CAR-NK cells did not expand in vivo at the time of relapse.

Analysis of Serum Cytokines

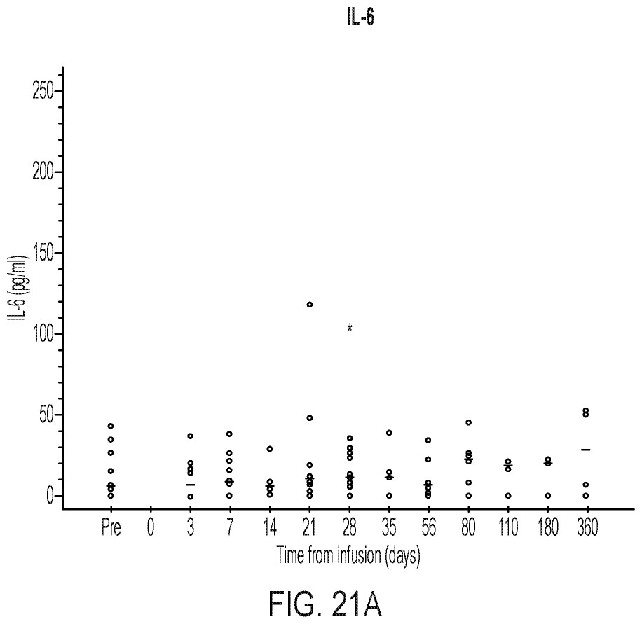

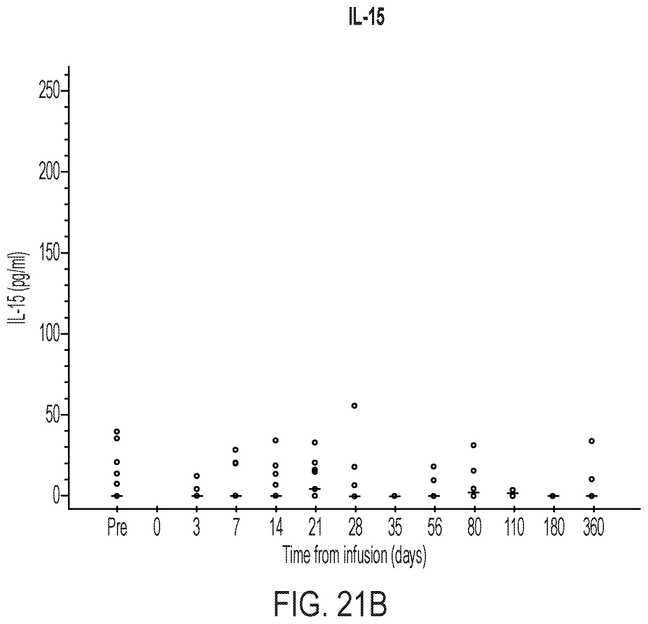

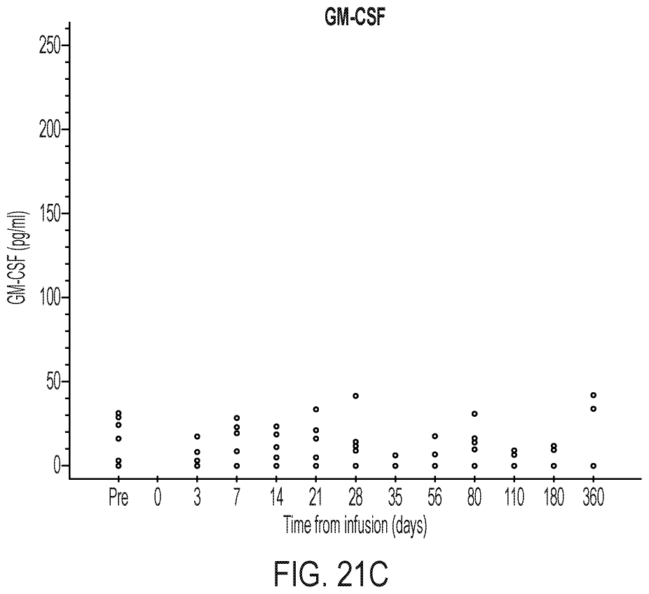

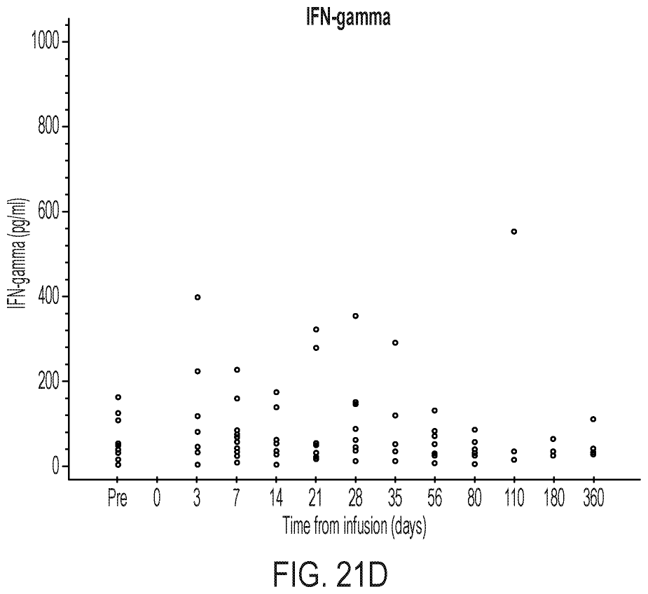

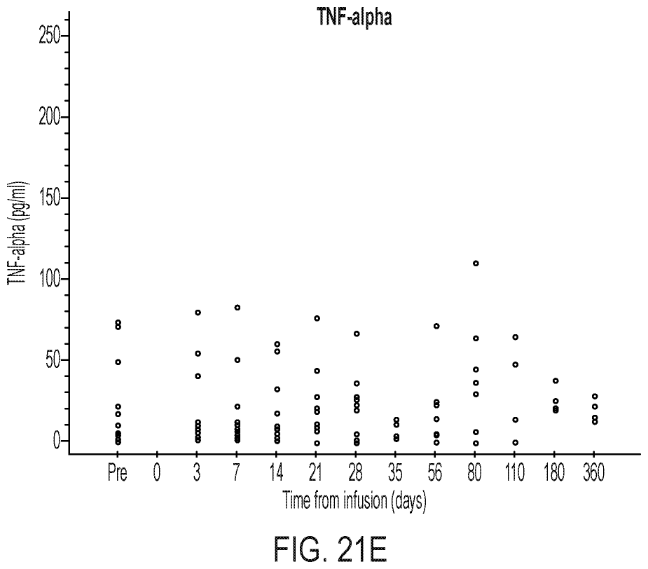

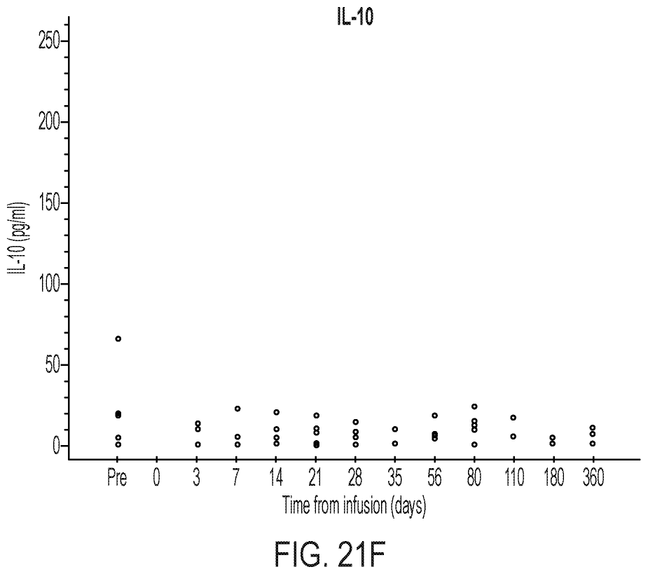

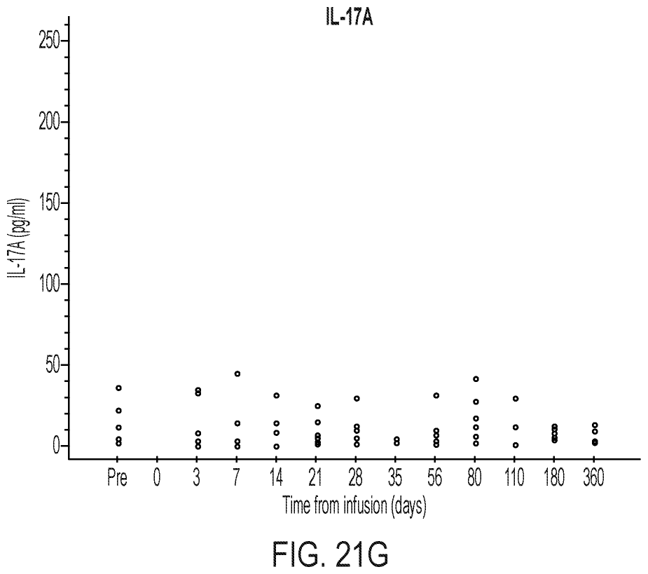

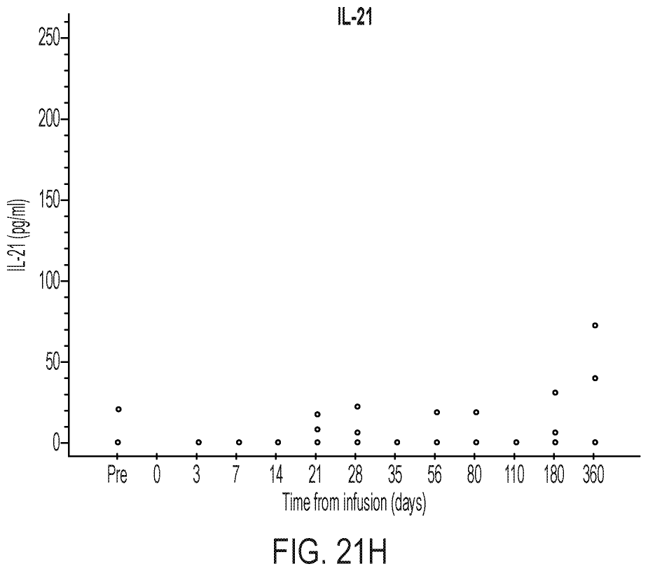

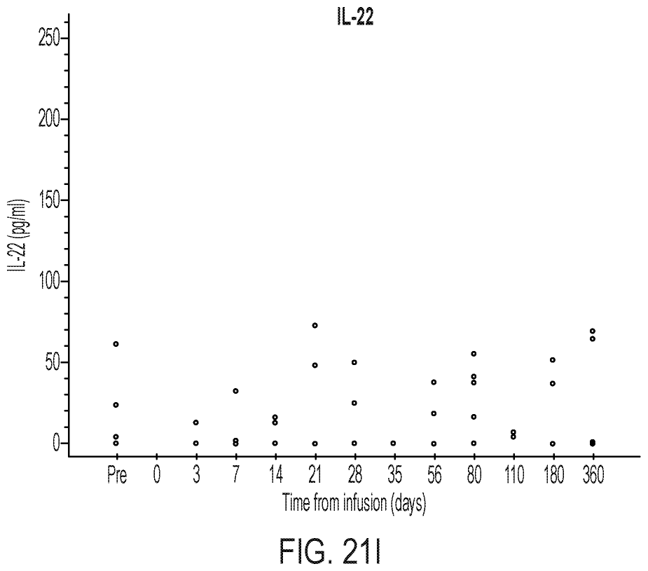

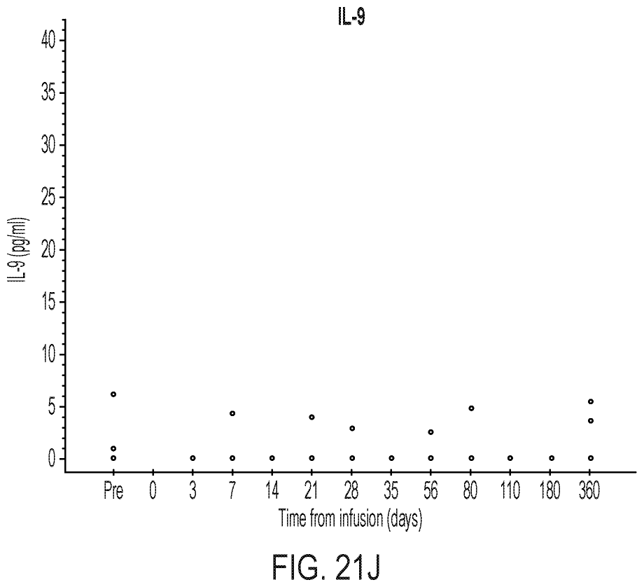

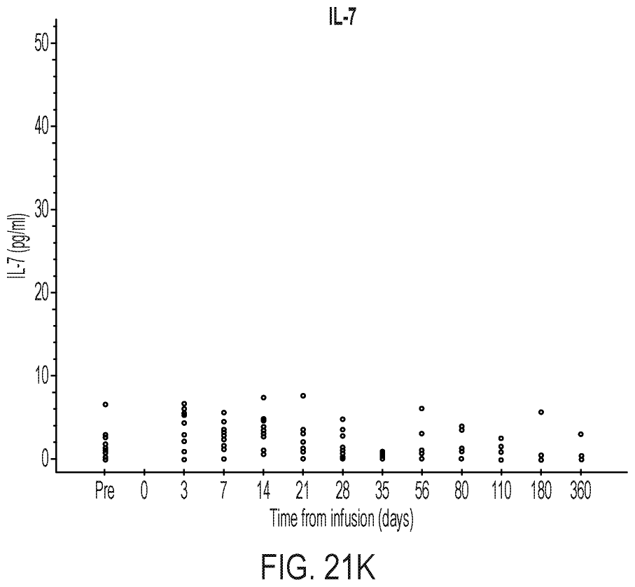

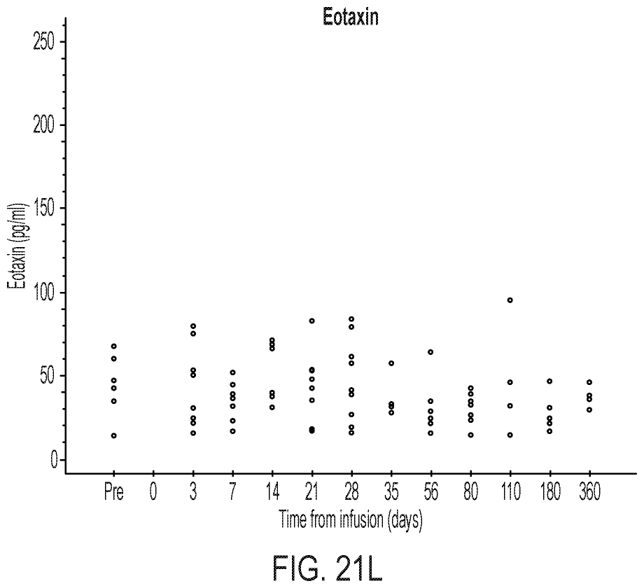

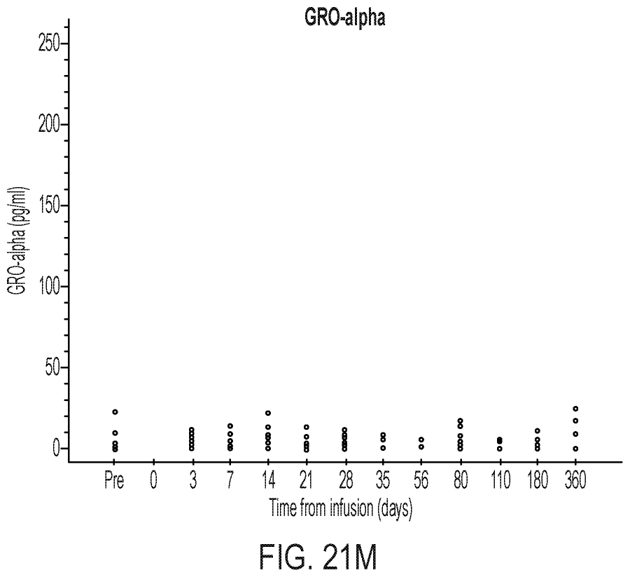

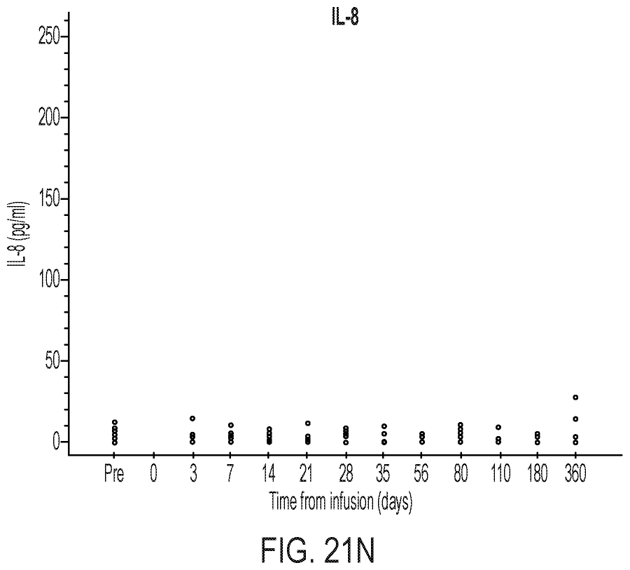

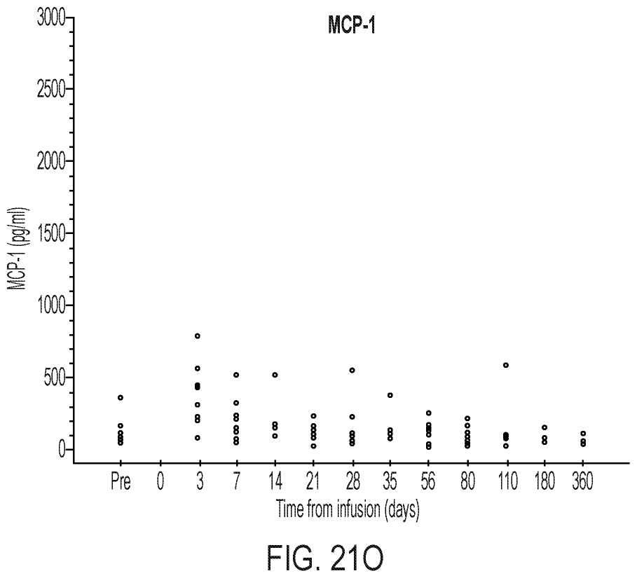

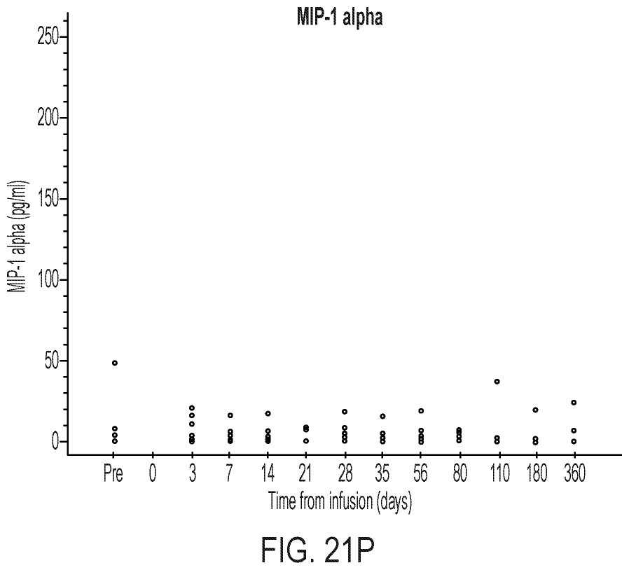

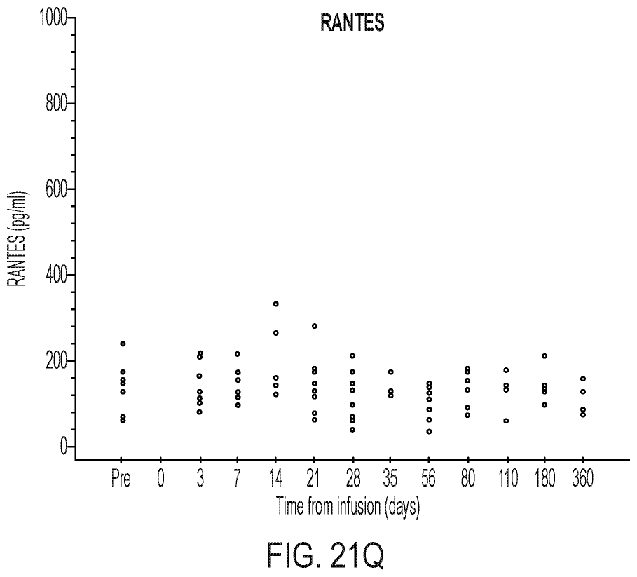

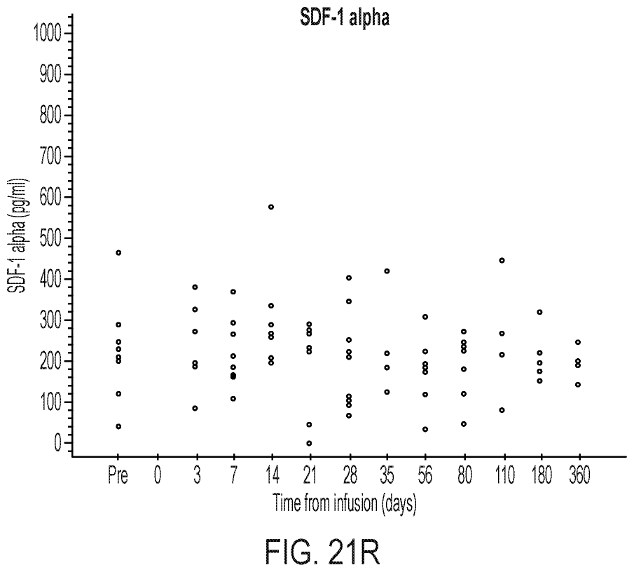

The supernatants from serial peripheral-blood samples were measured for inflammatory cytokines as well as for interleukin-15, which was encoded by the retroviral vector that was used to produce the CAR-NK cells. There was no observed increase in the levels of inflammatory cytokines (e.g., interleukin-6 and tumor necrosis factor α) as compared with the baseline levels, nor was there an increase in the systemic levels of interleukin-15 over pretreatment values, which indicated that interleukin-15 was not released to substantial systemic levels by CAR-NK cells in the peripheral blood after infusion (FIG. 21).

Induction of Alloimmune Antibody Responses Against the Donor

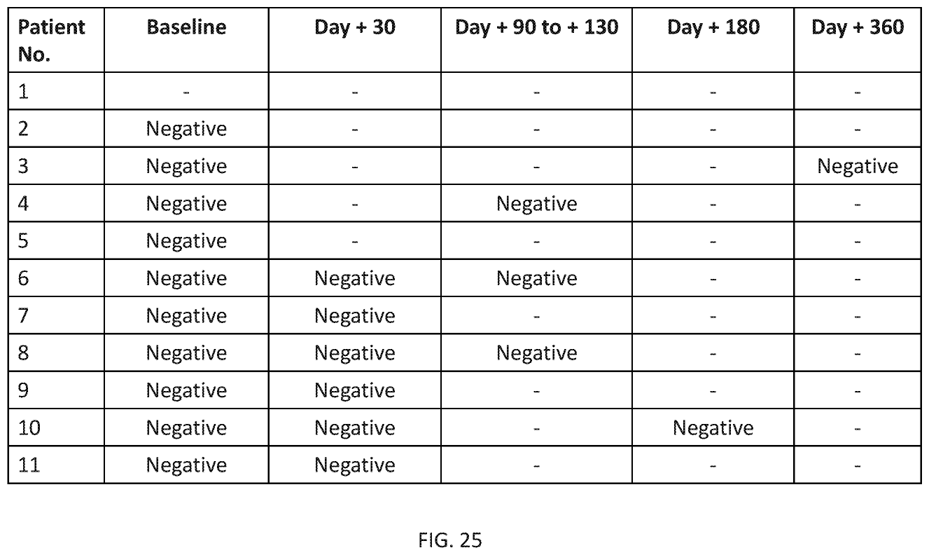

All of the patients received HLA-mismatched CAR-NK products. Patients 1 through 9 received a product with partial matching at 4 of 6 HLA molecules, whereas Patients 10 and 11 were recipients of non-HLA-matched CAR-NK cells. Thus, the inventors monitored for the induction of donorspecific HLA antibodies. At all the time points when testing was performed, no antibody induction against the mismatched HLA alleles of the infused product was observed (FIG. 25). Host cellular responses were not assessed.

### Example 3—Supplementary Materials

CAR-NK Cell Manufacture

The preclinical development of iC9/CAR19/IL15 CAR-NK cells was described previously (Hoyos et al., 2010; Liu et al., 2018). The clinical CB units for CAR-NK cell production were obtained from the MD Anderson Cancer Center (MDACC) CB bank. The CAR-NK cells were manufactured in the MDACC GMP facility. Briefly, the cord unit was thawed and NK cells were purified by CD3, CD19 and CD14 negative selection (Miltenyi beads) and cultured in the presence of engineered K562 feeder cells expressing membrane-bound IL-21 and 4-1BB ligand and exogenous IL-2 (200 U/ml). On day 6, cells were transduced with a retroviral vector carrying a single chain variant fragment (scFv) against CD19, a CD28 transmembrane domain, and a CD28.CD3t signaling endodomain, in combination with the human IL15 gene and the inducible caspase-9 suicide gene. The three genes were linked together using 2A sequence peptides derived from foot-and-mouth disease virus, and cloned into the SFG retroviral vector to generate the iC9/CAR.19/IL15 retroviral vector (Hoyos et al., 2010; Liu et al., 2018). The cells were expanded for an additional 9 days and harvested for fresh infusion on day 15.

Study Design

A phase I-II clinical trial was conducted at the institution of the inventors, and the trial was designed to identify the optimal dose and assess the safety and efficacy of escalating doses of iC9/CAR19/IL15 CB-NK cells as treatment for relapsed/refractory CD19-positive malignancies. The dose was escalated using the sequentially adaptive phase I-II EffTox trade-off-based design (Thall and Cook, 2004; Thall et al., 2006; Thall et al., 2014). Dose limiting toxicity was defined as occurrence of CRS within 2 weeks of the cell infusion that required transfer to the intensive care unit or the development of grade III-IV acute GVHD within 40 days of the infusion or grade 3-5 allergic reaction related to the NK-CAR cell infusion. For the purpose of the EffTox model, efficacy was defined as the patient being alive and in at least a partial remission at day 30 post CAR-NK cell infusion.

All adverse events in the first 40 days after infusion, irrespective of their attribution to the CAR-NK cell therapy, were collected and reported. From day 40 until 12 month after therapy, all adverse events deemed to be at least possibly related to CAR-NK cells were collected and reported. In addition, all patients are enrolled on an IRB-approved long-term follow-up study (for 15 years). The EffTox dose acceptability rules contemplated an upper limit of the probability of dose limiting toxicity of 0.50 (based on the CAR-T cell experience we anticipated that 20% of the patients will develop dose limiting CRS) and a lower limit of the probability of efficacy of 0.25. The three equivalent trade-off probability pairs used for computing the trade-off contours were (0.35, 0), (0.55, 0.30), (1, 0.075). The prior hyper-parameters were computed based on the assumed prior means Prob(Toxicity dose)=0.35, 0.40, 0.45 and Prob(Efficacy dose)=0.15, 0.20, 0.25 respectively with the overall prior effective sample size=1. One can treat a maximum of 36 patients in up to 3 cohorts of size 12 starting at the lowest dose level (10⁵ cell/kg); subsequent doses were chosen by the EffTox method, and no untried dose level was skipped when escalating. The EffTox design was implemented using the MDACC Department of biostatistics Clinical Trial Conduct website https://biostatistics.mdanderson.org/ClinicalTrialConduct/.

Clinical Trial Amendments and Patient Enrolment

Between June 2017 and February 2019, 15 consecutives patients were enrolled in the protocol (4 were screen failures and 11 received the therapy). Patients were enrolled sequentially with a staggering interval of 14 days from the day of CAR NK infusion to the start of the preparative regimen for the next patient within each cohort, as well as a 2-week interval as the dose was escalated to the next level. In March 2019 the dose finding portion of the study was considered to be complete and the protocol was amended to allow for repeated CAR-NK cell infusions at the 10⁷ cells/kg dose. Examples 2 and 3 of this disclosure report on the 11 patients treated with a single infusion of CAR-NK cells on the dose finding part of the study. The data cutoff for this report was April 2019. The clinical trial was designed to follow the patients for 12 months, after which patients were followed on an IRB-approved long term follow up study for patients treated with genetically-modified cell products. All patients consented for participation in the long term follow up study.

Response Assessments

Bone marrow examinations and PET-CT imaging were performed at 4, 8, 12, 16, 26, 48 and 52 weeks after the infusion and more frequently if clinically indicated. Responses were defined using the Lugano and iWCLL criteria for NHL and CLL patients, respectively (Cheson et al., 2014; Hallek et al., 2008; Hallek et al., 2018). All bone marrow samples were evaluated for MRD status using 6 color flow cytometry with a sensitivity of 10-4 nucleated cells or better in the MDACC CLIA-certified hematopathology laboratory. Patients were considered MRD negative if they had at least two consecutive negative assessments.

Criteria for response assessment of Hodgkin and Non-Hodgkin Lymphoma (Lugano criteria) (Cheson et al., 2014)

Complete Response

| | PET-CT-Based Response | CT-Based Response |
|---|---|---|
| Lymph nodes and | Complete metabolic response: | Complete radiologic |
| extralymphatic | Score 1, 2, or 3_ with or without a residual mass on | respomse (all of the |
| sites | SPS† It is recognized that in Waldeyar's ring or | following) |
| | extranodal sites with high physiologic uptake or | Target nodes/nodal |
| | with activation within spleen or marrow (eg, with | masses must regress |
| | hemotherapy or myeloid colony-stimulating | to <1 5 cm in |
| | factors). uptake may be greater than normal | LDi No extralymphatic |
| | mediastinum and/or liver. In this circumstance, | sites of disease |
| | complete metabolic response may be interred if | |
| | uptake at sites of initial involvement is no greater | |
| | than surrounding normal tissue even if the tissue | |
| | has high physiologic uptake | |
| Nonmeasured lesion | Not applicable | Absent |
| Organ enlargement | Not applicable | Regress to normal |
| New lesions | None | None |
| Bone marrow | No evidence of FDG-avid | Normal by morphology; |
| | disease in marrow | if indeterminate. |
| | | IHC negative |

Partial Response

| | PET-CT-Based Response | CT-Based Response |
|---|---|---|
| Lymph nodes and | Partial metabolic response | Partial remission (all |
| extralymphatic | Score 4 or 5† with reduced | of the following) >50% |
| sites | uptake compared with baseline | decrease in SPD of up to |
| | and residual mass(es) of any size | 6 target measurable nodes |
| | At interim, these findings | and extranodal sites |
| | suggest responding disease | When a lesion is too small |
| | At end of treatment, these | to measure on CT, assign |
| | findings indicate residual | 5 mm × 5 mm as the default |
| | disease | value When no longer visible, |
| | | 0 × 0 mm |
| | | For a node >5 mm × 5 mm. |
| | | but smaller than normal. use |
| | | actual measurement for calculation |
| Nonmeasured lesion | Not applicable | Absent/normal, regressed, |
| | | but no increase |
| Organ enlargement | Not applicable | Spleen must have regressed by |
| | | 50% in length beyond normal |
| New lesions | None | None |
| Bone marrow | Residual uptake higher than uptake | Not aplicable |
| | in normal marrow but reduced | |
| | compared with baseline (diffuse | |
| | uptake compatible with reactive | |
| | changes from chemotherapy allowed). | |
| | If there are persistent focal | |
| | changes in the marrow in the | |
| | context of a nodal response, | |
| | consideration should be given to | |
| | further evaluation with MRI or | |
| | biopsy or an interval scan | |

No Response or Stable Disease

| | PET-CT-Based Response | CT-Based Response |
|---|---|---|
| Target nodes/ | No metabolic response | Stable disease <50% |
| nodal masses, | Score 4 or 5 with no | decrease from baseline |
| extranodal | significant change in FDG | in SPD of up 6 dominant, |
| lesions | uptake from baseline at | measurable nodes and |
| | interim or end of treatment | extranodal sites: no |
| | | criteria for progressive |
| | | disease are met |
| Nomeasured | Not applicable | No increase consistent |
| lesion | | with progression |
| Organ | Not applicable | No increase consistent |
| enlargement | | with progression |
| New lesions | None | None |
| Bone marrow | No change from | Not applicable |
| | baseline | |

Progressive Disease

| | PET-CT-Based Response | CT-Based Response |
|---|---|---|
| Individual target | Progressive metabolic disease | Progressive disease requires |
| nodes/nodal | Score 4 or 5 with an | at least 1 of the following |
| masses | increase in intensity of uptake | PPD progression: |
| Extranodal | from baseline and/or | An individual node/lesion must |
| lesions | New FDG-avid foci consistent | be abnormal with: LDi >1.5 cm and |
| | with lymphoma at | Increase by >50% from PPD nadir and An |
| | interim or end-of-treatment | increase in LDi or SDi from nadir |
| | assessment | 0.5 cm for lesions <2 cm |
| | | 1.0 cm for lesions >2 cm |
| | | In the setting of splenomegaly, |
| | | the splenic length must increase |
| | | by >50% of the extent of its prior |
| | | increase beyond baseline (eg, a |
| | | 15-cm spleen must increase |
| | | to >16 cm). If no prior splenomegaly, |
| | | must increase by at least 2 cm |
| | | from baseline |
| | | New or recurrent splenomegaly |
| Nonmeasured | None | New or clear progression of |
| lesion | | preexisting nonmeasured lesions |
| New lesions | New FDG-avid foci consistent | Regrowth of previously resolved |
| | with lymphoma rather than | lesions A new node >1.5 cm in any axis |
| | another etiology (eg, infection, | A new extranodal site >1.0 cm is any axis, if <1.0 |
| | inflammation). If uncertain | cm in any axis, its presence must be unequivocal |
| | regarding etiology of new | and must be attributable to lymphoma |
| | lesions, biopsy or interval | Assessable disease of any size unequivocally |
| | scan may be considered | attributable to lymphoma |
| Bone marrow | New or recurrent FDG-avid foci | New or recurrent involvement |

Abbreviations: 5PS, 5-point scale; CT, computed tomography; FDG, fluorodeoxyglucose; IHC, immunohistochemistry; LDi, longest transverse diameter of a lesion; Mill, magnetic resonance imaging; PET, positron emission tomography; PPD, cross product of the LDi and perpendicular diameter; SDi, shortest axis perpendicular to the LDi; SPD, sum of the product of the perpendicular diameters for multiple lesions. *A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (eg, GI tract, liver, bone marrow), FDG uptake may be greater than in the mediastinum with complete metabolic response, but should be no higher than surrounding normal physiologic uptake (eg, with marrow activation as a result of chemotherapy or myeloid growth factors). †PET 5PS: 1, no uptake above background; 2, uptake <mediastinum; 3, uptake >mediastinum but <liver; 4, uptake moderately >liver; 5, uptake markedly higher than liver and/or new lesions; X, new areas of uptake unlikely to be related to lymphoma.

iwCLL response criteria for CLL (Hallek et al., 2018)

Complete Remission

CR requires all of the following criteria:

1. Peripheral blood lymphocytes (evaluated by blood and differential count) <4×109/L.

2. Absence of significant lymphadenopathy by physical examination. In clinical trials, a CT scan of the neck, abdomen, pelvis, and thorax is desirable if previously abnormal. Lymph nodes should be <1.5 cm in longest diameter. Once this is determined, further imaging should not be required until disease progression is apparent by clinical examination or on blood testing.

3. No splenomegaly or hepatomegaly by physical examination. In clinical trials, a CT scan of the abdomen should be performed at response assessment and should show no evidence for lymphadenopathy and splenomegaly.

4. Absence of disease-related constitutional symptoms.

5. Neutrophils ≥1.5×10⁹/L.

6. Platelets ≥100×10⁹/L.

7. Hemoglobin ≥11.0 g/dL (without red blood cell transfusions).

Some patients fulfill all the criteria for a CR but have a persistent anemia, thrombocytopenia, or neutropenia apparently unrelated to CLL, but related to drug toxicity. These patients should be considered as a different category of remission, CR with incomplete marrow recovery (CRi).

Partial Remission

To define a partial remission, at least 2 parameters of group A and 1 parameter of group B need to improve, if previously abnormal (Table below). If only 1 parameter of both groups A and B was abnormal before therapy, only 1 needs to improve.

| Group A | | | | | | |
|---|---|---|---|---|---|---|
| Lymph | Liver and/or | Constitutional | Lymphocyte | Group B | | |
| nodes | spleen size | symptoms | count | Platelet count | Hemoglobin | Marrow |
| Decrease ≥50% | Decrease ≥50% | Any | Decrease ≥50% | ≥100 × 10⁹/L or | ≥11 g/dL or | Presence of CLL cells, |
| (from baseline) | (from baseline) | | from baseline | increase ≥50% | increase ≥50% | or of |
| | | | | over baseline | over baseline | B-lymphoid nodules |

### Progressive Disease

Progressive disease during or after therapy is characterized by at least 1 of the following, when compared with nadir values:

1. Appearance of any new lesion such as enlarged lymph nodes (>1.5 cm), splenomegaly, hepatomegaly, or other organ infiltrates.

2. An increase by >50% in greatest determined diameter of any previous lymph node (>1.5 cm).

3. An increase in the spleen size by ≥50% or the de novo appearance of splenomegaly. In the setting of splenomegaly, the splenic length must increase by ≥50% of the extent of its prior increase beyond baseline. If no prior splenomegaly was observed at baseline or if splenomegaly has resolved with treatment, the spleen must increase by at least 2 cm from baseline.

4. An increase in the liver size of ≥50% of the extent enlargement of the liver below the costal margin defined by palpation, or the de novo appearance of hepatomegaly.

5. An increase in the number of blood lymphocytes by 50% or more with at least 5×109/L B lymphocytes.

6. Transformation to a more aggressive histology (Richter syndrome)

7. Occurrence of cytopenia (neutropenia, anemia, or thrombocytopenia) directly attributable to CLL and unrelated to autoimmune cytopenias.

a. Decrease of Hb levels ≥2 g/dL or <10 g/dL

b. Decrease of platelet counts ≥50% or <100×10⁹/L

8. Increase of CCL cells in the bone marrow ≥50% in successive biopsies

### Stable Disease

Patients who have not achieved a CR or a partial remission, and who have not exhibited PD, will be considered to have stable disease

### Relapse

Relapse is defined as evidence of disease progression in a patient who has previously achieved the above criteria of a CR or partial remission for ≥6 months.

CAR-NK Cell Cytotoxicity Against Primary CLL Targets

PBMCs from 4 different CLL patients were thawed and pre-activated overnight with CD40L (2 ng/ml) in SCGM media at a concentration of 2×10⁶ PBMCs/ml in a humidified incubator at 37° C./5% CO₂. A 4 h ⁵¹Cr-release assay was performed in v-bottomed 96-well plates. Briefly, 0.5×10⁶ CLL cells were resupended in 1 ml of SCGM and labeled with 100 microcuri of ⁵¹Cr for 2 h in a humidified incubator at 37° C./5% CO₂. After labeling, cells were washed two times with PBS and resuspended in SCGM and then used as targets for the assays. Paired non-transduced (NT) and CAR-transduced NK cells (CAR-NK) were used as effectors, at different effector-totarget cell ratios (E: T). Percent specific lysis was defined as [(mean of the test wells)−(mean of the spontaneous release wells)/(mean of maximal release wells)−(mean of the spontaneous release wells)]×100. Student paired t-test was used to calculate the statistical significance.

Analysis of Perforin-Dependent CAR-NK Cell Cytotoxicity Against Primary CLL Targets

Concanamycin A (CMA) was used to prevent the maturation of perforin and to deplete NK cells of active perforin (Kataoka et al., 1996). Briefly, 2×10⁶ CD19-CAR transduced NK cells were treated with 100 nM of CMA (Fisher) or DMSO (Sigma) as a vehicle control for 2 hours in a humidified incubator at 37° C./5% CO₂. Perforin expression in CAR-NK cells was assessed by flow cytometry using a monoclonal antibody against perforin (dG9 clone from BioLegen (Makedonas et al., 2009). A change in perforin expression was defined by comparing perforin MFI in CD56+ CAR-NK cells in the presence or absence of CMA. The cytotoxicity of CAR NK cells that were pre-treated with or without CMA against primary CLL targets was determined using ⁵¹Cr release assay as described above. Student's paired t-test was used to calculate the statistical significance.

qPCR

Genomic DNA was extracted using QIAamp DNA Blood Mini Kit (Qiagen), following the manufacturer's recommendation. Copies of vector transgene per micrograms genomic DNA was determined by quantitative PCR (qPCR) using Applied Biosystems 7500 Fast Real-Time PCR System. The amplified targets were detected in real time using TaqMan® Universal PCR Master Mix and a DNA-based, custom designed Applied Biosystems™ TaqMan® MGB (minor groove binder) probe that incorporates a 5′ reporter (FAM) and a 3′ non-fluorescent quencher (NFQ), and quantified using a standard curve. The quantified copies of vector transgene per reaction are reported as copies per 1 μg DNA. Fluorescence data was analyzed using 7500 Software v2.3.

Examples of Primer Probe Sequences:

Forward Primer
(SEQ ID NO: 7)
GAACAGATTATTCTCTCACCATTAGCA
Reverse Primer
(SEQ ID NO: 8)
AGCGTATTACCCTGTTGGCAAA
TaqMan FAM-MGB Probe
(SEQ ID NO: 9)
CCTGGAGCAAGAAG

The primers and probe were custom designed and synthesized by Thermo Fisher Scientific.

Analysis of Serum Cytokines

Serum from serial peripheral blood samples collected before and after CAR-NK infusion were measured for cytokines using the Procartaplex kit from Thermofisher (Vienna, Austria) following the manufacturer's instructions.

Phenotyping and Tracking of CAR-NK Cells by Multiparameter Flow Cytometry

To determine the persistence of CB-derived CAR-NK cells in the peripheral blood and their trafficking to the bone marrow and lymph nodes, a two-step strategy was used. First, the inventors took advantage of the existing HLA-mismatch between the patient and the donor to identify the infused cord-blood derived NK-cells; next a CAR-specific antibody was used to identify the CAR-expressing cells within the donor NK cell population. Briefly, a flow-chimerism assay was developed using fluorochrome-conjugated antibodies against the mismatched HLA alleles. In addition, an anti-CAR antibody (109606088/Jackson Immuno Rsch) directed against the CH2-CH3 domain of the human IgG hinge was used in the construct as a second method to detect the CAR NK cells. Cells were stained with a live/dead dye (Tonbo BioScience: 13-0868-T100) in 1 ml of PBS, for 20 minutes at room temperature. The cells were then washed twice by centrifugation at 400×g at room temperature for 5 minutes with flow buffer containing PBS and 1% heat-inactivated FBS. Next, the cells were stained with AF-647-conjugated anti-CAR (109606088/Jackson Immuno Rsch) antibody for 20 minutes at 4° C. Cells were washed and stained with the relevant anti-HLA antibody at 4° C. for 10 minutes. Cells were then incubated with a cocktail of fluorescent-tagged antibodies containing CD19 PE-Cy5, CD20 FITC, CD3 APC-Cy7, CD14 BUV395, CD33 BUV395 (all BD Biosciences), CD45 BV510 (BeckMan Coulter), CD56 PE-TX Red (BeckMan Coulter) and CD16 BV650 (Biolegend) for 15 minutes at room temperature. Cells were then washed by centrifugation at 400×g and fixed with 1% Paraformaldehyde. Flow cytometry was performed on a BD LSRFortessa X-20 instrument, and data were analyzed with the use of FlowJo software, version 10.0.8 (TreeStar). The gating strategy for the detection of HLA-positive CARpositive NK cells is shown in FIG. 16.

Detection of CAR-NK in Lymph Node Samples

Fine needle lymph node biopsy samples were collected in PBS. Single cell suspension of the samples was prepared by mincing the sample between two frosted end slides. The cell suspension was filtered through a 50 micron mesh, spun down, counted and 1×106 cells were stained with live dead dye in PBS. After viability staining, cells were washed once in PBS with 1% FBS, and stained with AF-647-conjugated anti-CAR (109606088/Jackson Immuno Rsch) antibody (4° C., 20 minutes) in PBS-FBS buffer. Next, cells were washed in PBS-FBS buffer, and sequentially stained with an HLA-specific antibody (4° C., 10 minutes) followed by a cocktail of antibodies against CD19 PE-Cy5, CD20 FITC, CD3 APC-Cy7, CD14 BUV395, CD33 BUV395 (all BD Biosciences), CD45 BV510 (BeckMan Coulter), CD56 PE-TX Red (BeckMan Coulter) and CD16 BV650 (Biolegend) Cells were fixed in 2% paraformaldehyde and analyzed on the X20 Fortessa analyzer.

Donor-Specific Antibody (DSA) Measurement

Ten of the eleven patients were screened for the presence of donor-specific anti-HLA antibodies before and at multiple time points after CAR-NK infusion. If the screen was positive the specificity of the antibody was determined using semi-quantitative solid phase antibody detection on a Luminex® platform.

### Example 4—an Example of a Dose Escalation Study Phase I/II of Umbilical Cord Blood-Derived CAR-Engineered NK Cells in Conjunction with Lymphodepleting Chemotherapy in Patients with Relapsed/Refractory B-Lymphoid Malignancies

The present example concerns determination of the safety and efficacy of CAR.CD19-CD28-zeta-2A-iCasp9-IL15-transduced CB-NK cells in patients with relapsed/refractory CD19+B lymphoid malignancies. This example allows for assessment of the overall response rate (complete and partial response rates), quantification of the persistence of infused allogeneic donor CAR-transduced CB-derived NK cells in the recipient, and performance of comprehensive immune reconstitution studies.

Background

The present example describes a clinical trial for investigating novel immunotherapeutic strategies, using engineered natural killer (NK) cells to improve the tumor-free survival of patients with relapsed or refractory CD19+B-cell malignancies. There are an annual average of 69,740 new cases of non-Hodgkin lymphoma (NHL), 15,680 new cases of chronic lymphocytic leukemia (CLL) and 6070 new cases of acute lymphoblastic leukemia (ALL) in the United States, with estimated annual death rates of 19,020, 4580 and 1,430 respectively (http:/www.cancer.org). Overall survival (OS) is determined largely by disease stage at presentation and response to chemotherapy. Standard therapy for patients who relapse following frontline therapy is allogeneic hematopoietic stem cell transplantation (HSCT). The expected OS for patients in 2nd complete remission is 25% based on chemotherapy-sensitivity at the time of HSCT. Thus, there is an urgent and unmet need to develop new therapies for patients with advanced B-lineage malignancies, especially because relapse after allogeneic HSCT is usually fatal.

Chronic lymphocytic leukemia (CLL) is the most common form of adult leukemia in the United States, accounting for 25% of all leukemias. There are more than 15,000 new cases of CLL and 4,500 deaths from CLL every year in the United States. The natural history of the disease is diverse. Patients with only lymphocytosis have a median survival greater than of 10 years, whereas those with evidence of marrow failure manifested by anemia or thrombocytopenia have a median survival of only 2-3 years. Since no treatment has been shown to be curative, nor is there objective evidence that a specific treatment prolongs survival, treatment is delayed (Cheson and Cassileth, 1990). The NCI-sponsored CLL Working Group proposed the following indications for initiating treatment: 1) weight loss of more than 10% over the preceding 6 months; 2) extreme fatigue attributable to progressive disease; 3) fever or night sweats without evidence of infection; 4) worsening anemia (Rai stage III) or thrombocytopenia (Rai stage IV); 5) massive lymphadenopathy (>10 cm) or rapidly progressive lymphocytosis (lymphocyte doubling time <6 months); or 6) prolymphocytic or Richter's transformation. Current treatments for newly diagnosed CLL include chemotherapy and antibody therapy either alone or in combination. A variety of novel approaches such as targeted therapy using ibrutinib for treating CLL are being developed (Burger et al., 2015; Byrd et al., 2015), but the disease is not yet curable. Moreover, even after complete responses, immunological abnormalities and minimal residual disease remain in most patients. Ultimately, chronic immunosuppression resulting in infectious complications occurs in 80% of CLL patients and is a major cause of mortality. Allogeneic stem cell transplantation may be curative in some patients with CLL, but success has been limited, primarily due to the high incidence of mortality and morbidity associated with the procedure. Non-myeloablative allogeneic transplant regimens hold promise, but patient eligibility is limited by availability of matched sibling donors.

Historically the initial treatment of patients with CLL requiring treatment has been with an alkylating agent, particularly chlorambucil, alone or in combination with a corticosteroid. The overall response rate has been 50-70%; however, the complete remission rates are low (5-20%). Newer agents like purine analogues, particularly fludarabine, have higher response rate (Rai et al., 2000) as initial treatment. Randomized trials comparing alkylating agent-based therapy with single-agent fludarabine have shown a higher complete response rate and longer disease-free (Rai et al., 2000; Johnson et al., 1996) survival with the nucleoside analogue, but have not shown a survival advantage. Fludarabine was approved by the U.S. Food and Drug Administration (FDA) for the treatment of patients with B cell CLL who have not responded to or progressed during treatment with at least one alkylating agent-based regimen.

Combination regimens such as cyclophosphamide, fludarabine and Rituximab have been shown to improve response rates (Keating et al., 2005), but these regimens are highly immunosuppressive, and long-term benefit has not been demonstrated. Ibrutinib is a covalent inhibitor of Bruton's (Honigberg et al., 2010) tyrosine kinase (BTK), a member of the TEC tyrosine kinase family and a key enzyme in the B-cell receptor signaling pathway. Ibrutinib as monotherapy, as well as in combination with immunotherapy or chemotherapy, is a very effective therapy for lymphoid malignancies including CLL (Burger et al., 2015; Byrd et al., 2013). However, outcomes after ibrutinib failure are dismal, with only a 3.1 month survival after drug discontinuation (Jain et al., 2015).

Acute Lymphoblastic Leukemia. Allogeneic HCT is a curative approach for a select group of patients with ALL. Overall survival (OS) ranges from 30%-60% depending on the patients disease stage and risk profile at time of transplant (Fielding et al., 2009; Golstone et al., 2008). Increasingly, minimal residual disease (MRD), both before and after HCT, is becoming an important predictor for relapse (Gokbuget et al., 2012). In a series of 149 ALL patients transplanted in remission at MD Anderson Cancer Center, patients with MRD, measured by multi-parameter flow cytometric immunophenotyping (FCI) with a sensitivity of 0.01%, present at time of HCT had a shorter PFS compared to patients who were MRD negative, 28% vs. 47%, p=0.08 (4). Furthermore, among 135 patients who had MRD measured following HCT, 20 became positive for MRD, and 18 of these patients developed overt hematologic relapse within a median of 3.8 months (Zhou et al., 2014). Of note, among 32 patients with overt relapse following HCT, 41% did NOT have preceding MRD, suggesting that positive MRD post HCT essentially confirms eventual relapse, but negative MRD post HCT in a high-risk patient does not preclude relapse (Leung et al., 2012; Bar et al., 2014). The findings corroborate similar published studies. Patients transplanted beyond second remission routinely have a significantly lower PFS and OS rates. In a study of 97 patients (CR1 51, CR2 29, others 17) treated with busulfan and clofarabine chemotherapy conditioning following a matched sibling (MSD) or matched unrelated donor (MUD) transplant, patients in CR1 had a significantly better disease free survival (DFS) compared with others. For patients in CR1, the 2-yr DFS rate was 61% with 9/51 patients relapsing at median 9 months, the 2-yr DFS rate was 40% for CR2, with 10/29 relapsing at median 3 months, and for patients with more advanced disease, the 2-yr DFS rate was 33% with 3/17 progressing at a median of 3 months. Data from the Center for International Blood and Marrow Transplant Research (CIBMTR) corroborate the findings. Between 1996 and 2001, in patients less than 20 years-old, OS ranges from 25% for patients transplanted beyond first remission to 50% for sibling transplants in first remission. Similarly, in adult patients, greater than 20 years-old, the best outcome is noted in sibling transplants done in first remission with OS of 60%, as compared to 35% if transplants are performed beyond CR1 (CIBMTR Registry). No effective treatment options exist for patients who relapse following HCT. Multiple published series report less than 10% survival for these patients, regardless of the treatment modality used, with a median survival of 2-3 months (Fielding et al., 2009; Poon et al., 2013). To date, the most common strategy employed to reduce relapse rates after HCT has usually involved some form of immune manipulation, ranging from donor lymphocyte infusion (DLI) to second transplant (Sullivan et al., 1989; Poon et al., 2013; Bader et al., 2004). However, although it has been consistently shown that patients with B-ALL who develop graft-versus-host-disease (GVHD) have less risk for relapse (Appelbaum, 1997), DLI has not shown appreciable efficacy in this patient population; remission rates have been less than 10%, and have been associated with a high incidence of GVHD (Passweg et al, 1998). Of note, the best responses to DLI in ALL occur when the DLI is administered prophylactically to prevent relapse (Bader et al., 2004); this approach has been demonstrated in pediatric patients but no data for prophylactic DLI has been reported in adults. Thus, there is an unmet need for effective therapy for ALL patients at high risk for relapse following allogeneic HCT, with high risk defined as positive MRD and/or disease beyond first complete remission.

Non-Hodgkins Lymphoma (NHL). In the United States, B cell lymphomas represent 80-85% of cases reported. In 2013 approximately 69,740 new cases of NHL and over 19,000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer (SEER 2014). Diffuse Large B cell Lymphoma: Diffuse large B cell lymphoma (DLBCL) is the most common subtype of NHL, accounting for approximately 30% of NHL cases. There are approximately 22,000 new diagnoses of DLBCL in the United States each year. First line therapy for DLBCL typically includes an anthracycline-containing regimen with rituximab (Coiffier et al., 2002). The first line objective response rate and the complete response (CR) rate to R-CHOP (rituximab, cyclophosphamide, doxorubicin, vincristine, and prednisone) is approximately 80% and 50% respectively. However, approximately one-third of patients have refractory disease to initial therapy or relapse after R-CHOP (Sehn et al., 2005). For those patients who relapse after response to first line therapy, approximately 40-60% of patients can achieve a second response with additional chemotherapy. For patients who are young and fit, the goal of second line therapy is to achieve a response that will make the patient eligible for autologous stem cell transplant (ASCT). The standard of care for second-line therapy for transplant-eligible patients includes rituximab and combination chemotherapy such as RICE (rituximab, ifosfamide, carboplatin, and etoposide) or RDHAP (rituximab, dexamethasone, cytarabine, and cisplatin). In a large randomized trial of RICE versus RDHAP in transplant-eligible patients with DLBCL (the CORAL study) 63% of patients achieved an objective response to either regimen with a 26% CR rate. Patients who respond to second line therapy and who are considered fit enough for transplant receive consolidation with high-dose chemotherapy and ASCT. This combination can cure approximately 50% of transplanted patients (Gisselbrecht et al., 2010). Patients who fail ASCT have a very poor prognosis and no curative options. The majority of second line patients are not eligible for ASCT due to chemotherapy-refractory disease, age, or comorbidities such as heart, lung, liver, or kidney disease. Transplant-ineligible salvage patients do not have a curative option available to them. There is no standard definition of relapse/refractory DLBCL. This trial will enroll patients with chemo-refractory lymphoma, as evidenced by failure to achieve even a transient or partial response to prior biologic and combination chemotherapy or by early recurrence after ASCT.

Transformed Follicular Lymphoma (TFL). Follicular lymphoma (FL), a B cell lymphoma, is the most common indolent (slow-growing) form of NHL, accounting for approximately 20% to 30% of all NHLs. Some patients with FL will transform (TFL) histologically to DLBCL which is more aggressive and associated with a poor outcome. Histological transformation to DLBCL occurs at an annual rate of approximately 3% for 15 years with the risk of transformation continuing to drop in subsequent years. The biologic mechanism of histologic transformation is unknown. Initial treatment of TFL is influenced by prior therapies for follicular lymphoma but generally includes anthracycline-containing regimens with rituximab to eliminate the aggressive component of the disease (NCCN practice guidelines 2014). Treatment options for relapsed/refractory TFL are similar to those in DLBCL. Given the low prevalence of these diseases, no large prospective randomized studies in these patient populations have been 26 conducted. Patients with chemotherapy refractory disease have a similar or worse prognosis to those with refractory DLBCL.

Mantle cell lymphoma (MCL), an incurable subtype of B-cell lymphoma, accounts for 7% of all Non-Hodgkin lymphoma cases in the United States (Connors, 2013). Most MCL patients experience disease progression after frontline therapy, with a median overall survival of approximately 1-2 years after relapse; therefore, novel therapies for MCL are urgently needed. Ibrutinib, a first-in-class, once-daily, oral covalent inhibitor of Bruton's tyrosine kinase (BTK), was recently approved by the FDA to treat this disease. Although results in relapsed/refractory MCL were superior and unprecedented compared to other standard therapies (Wang et al., 2013), the vast majority of patients experience disease progression after single agent ibrutinib and die within 12 months (Wang et al., 2015; Cheah et al., 2015). Patients with relapsed/refractory MCL can achieve long-term remission and cure with immune cell-based therapies (Hamadani et al., 2013), including allogeneic stem cell transplantation (allo-SCT). Additionally we have reported promising results in selected MCL patients who received an autologous SCT following rituximab, BCNU, etoposide ara-C, melphalan (R-BEAM) therapy (Tam et al., 2009). However in MCL patients with ibrutinib resistant disease, the outcome of standard allogeneic and autologous transplants are suboptimal and clearly more effective therapies are needed. In summary, subjects who have refractory, aggressive B lymphoid malignancies have a major unmet medical need and novel treatments are warranted in these populations.

NK Cells:

Natural killer (NK) cells are an important component of the graft-versus-leukemia (GVL) response (Ruggeri et al., 2002; Savani et al., 2006), which is critical to preventing relapse after HSCT. Each mature NK cell expresses a wide array of activating and inhibitory killer immunoglobulin-like receptors (KIRs), which are specific for different HLA class-I molecules (Lanier, 2008; Yawata et al., 2008; Caligiuri, 2008). The ability of NK cells to recognize and kill malignant cells is governed by complex and poorly understood interactions between inhibitory signals resulting from the binding of inhibitory KIRs with their cognate HLA class-I ligands, and activating signals from activating receptors (Ruggeri et al., 2002; Caligiuri, 2008; Ljunggren et al., 1990). NK cell responses are mediated by two major effector functions: direct cytolysis of target cells and production of chemokines and cytokines. Through the latter mechanism (e.g., interferon-γ), NK cells participate in the shaping of the adaptive T cell response, possibly by a direct interaction between naïve T cells and NK cells migrating to secondary lymphoid compartments from inflamed peripheral tissues and by an indirect effect on dendritic cells (DC) (Martin-Fontecha et al., 2004; Krebs et al., 2009).

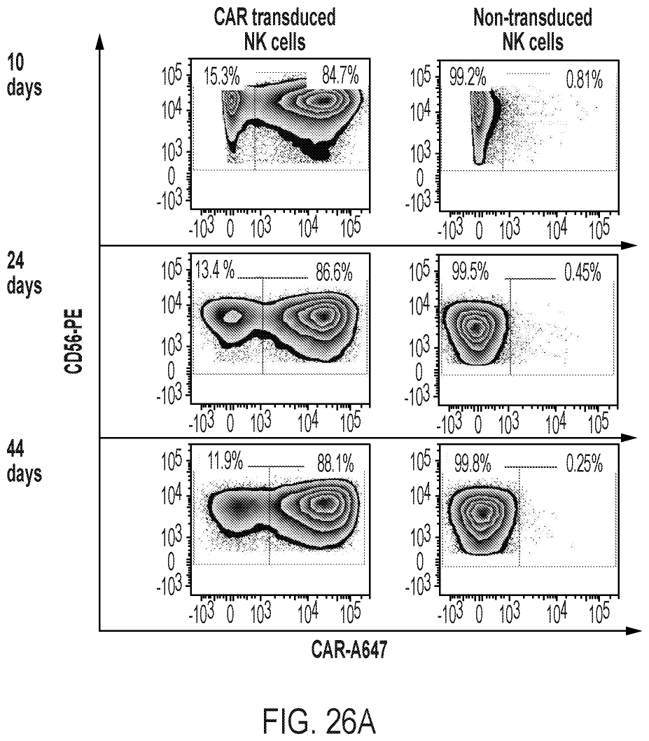

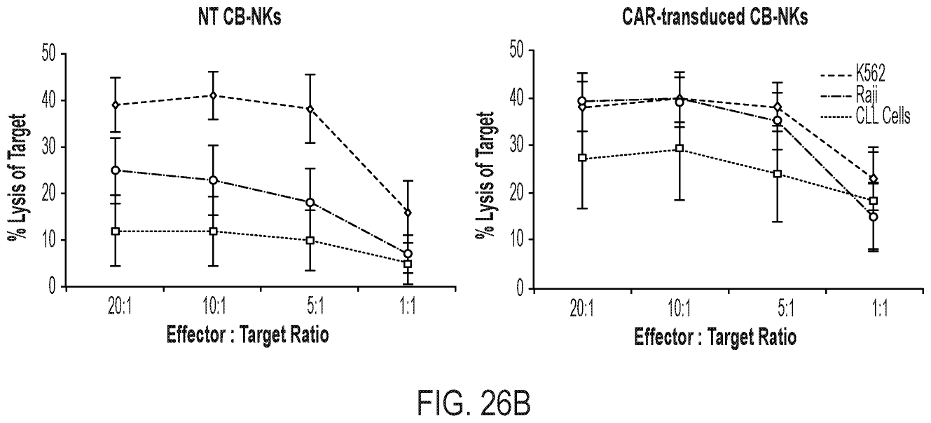

GMP-grade NK cell expansion from cord blood. Previous studies have largely used freshly obtained peripheral blood NK cells. The low number of circulating peripheral blood NK cells severely limits their therapeutic utility. The inventors have developed a system for ex vivo expansion of NK cells from cord blood (CB), which reliably generates clinically relevant doses of GMP grade CB-NK cells for adoptive immunotherapy, using GMP-grade K562-based artificial antigen presenting cells (aAPCs) expressing membrane bound IL-21, 4-1BB ligand, CD64 (FcγRI) and CD86 (clone 9.mbIL21) (Denman et al., 2012). Cord blood is a novel, attractive source of NK cells for cellular immune therapy. The cells are already collected, stored and immediately available. The cord blood donor can be optimally selected for HLA type, KIR gene expression and other factors. The methodology to generate CB NK cells has been approved by the FDA. Our current protocol yields a mean NK expansion of 3127 fold (range, 1640-4931 fold) (FIG. 26A), with very few CD3+ cells (mean, 4.50×10⁶) (FIG. 26B).

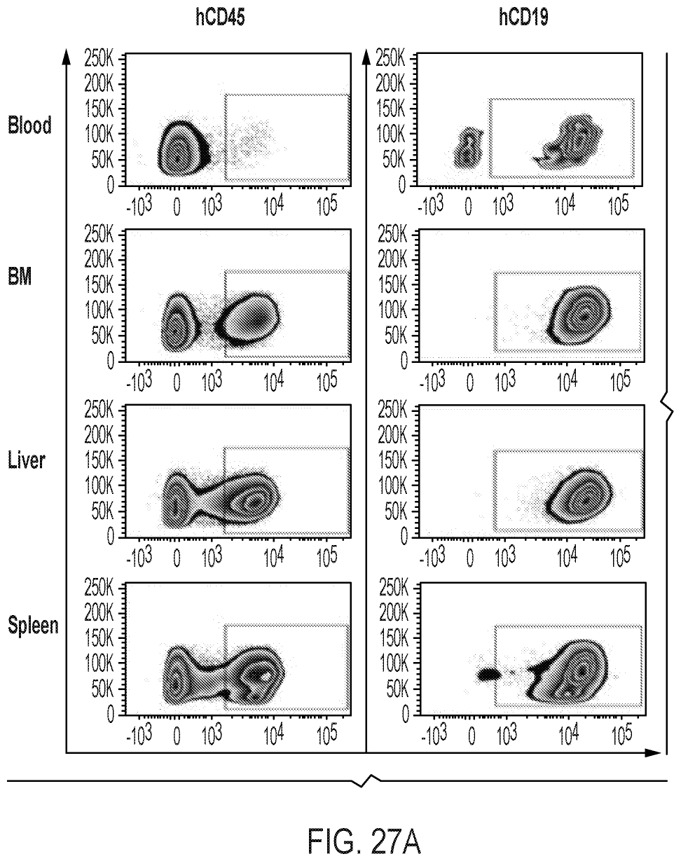

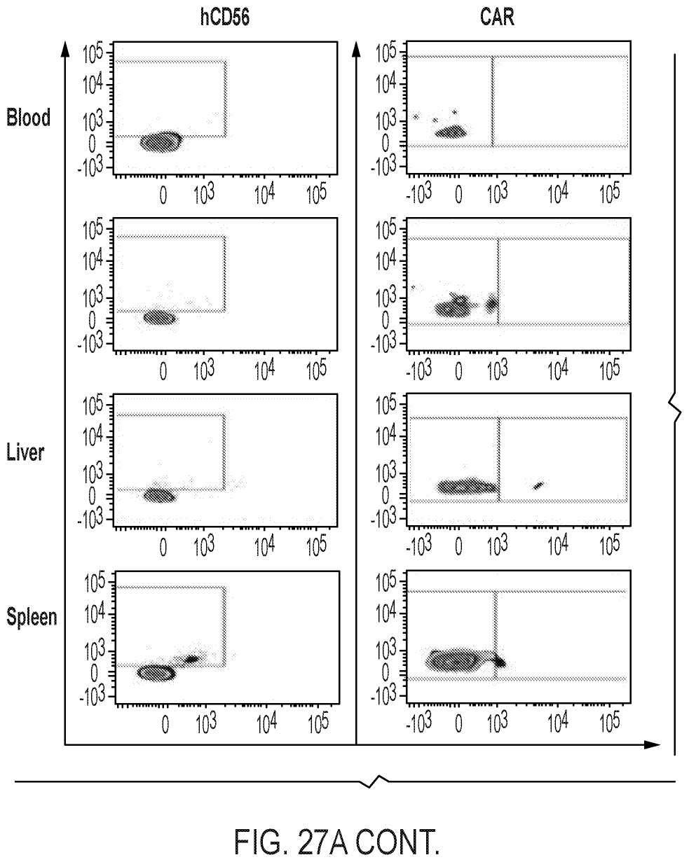

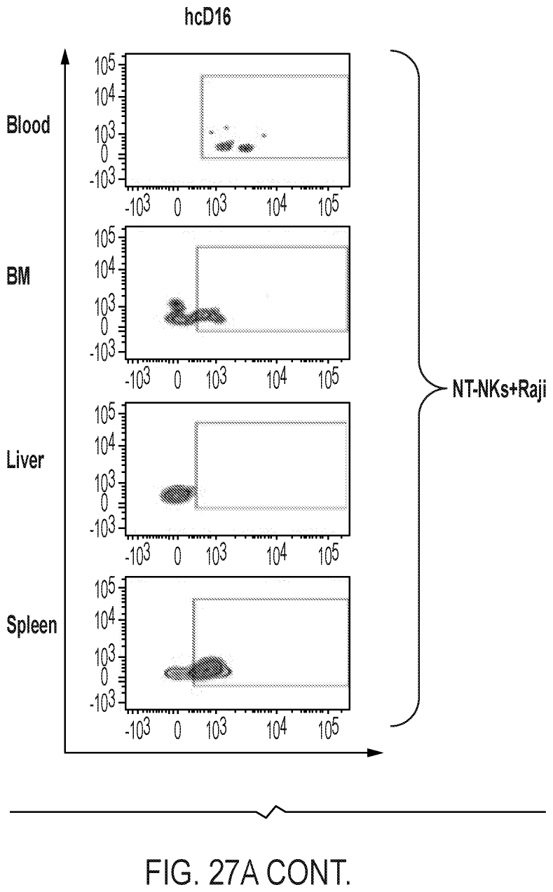

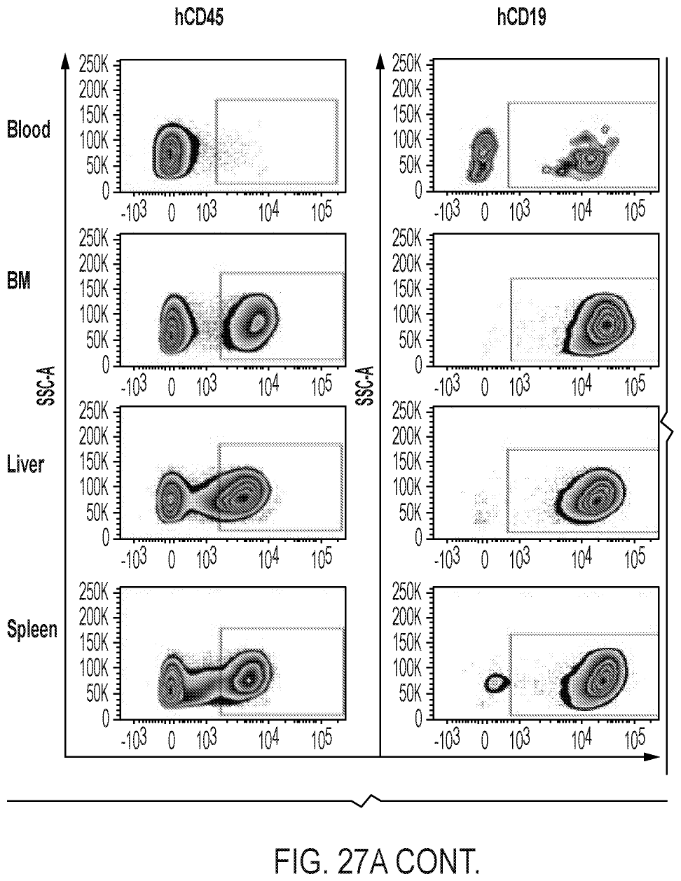

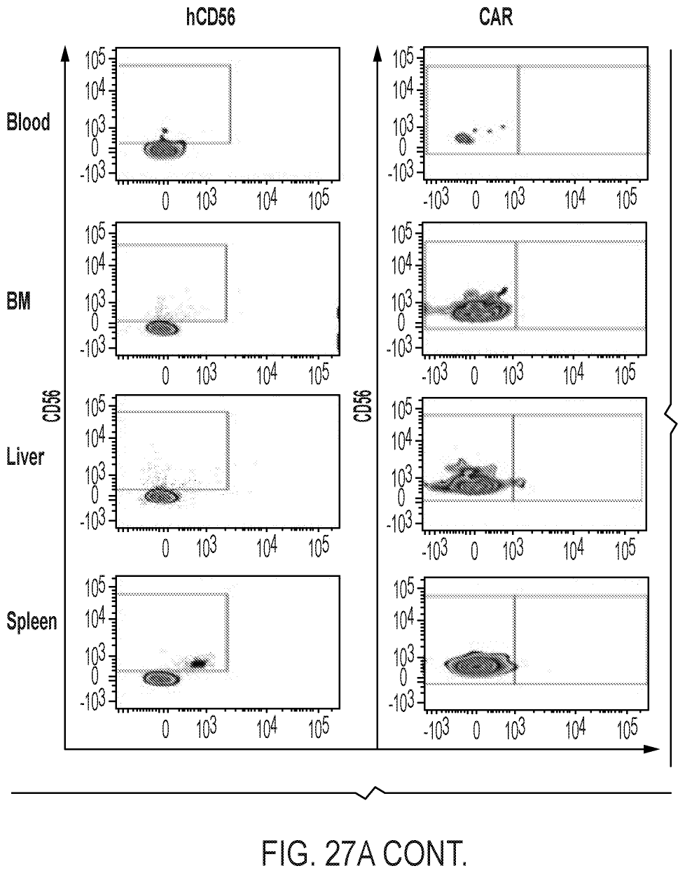

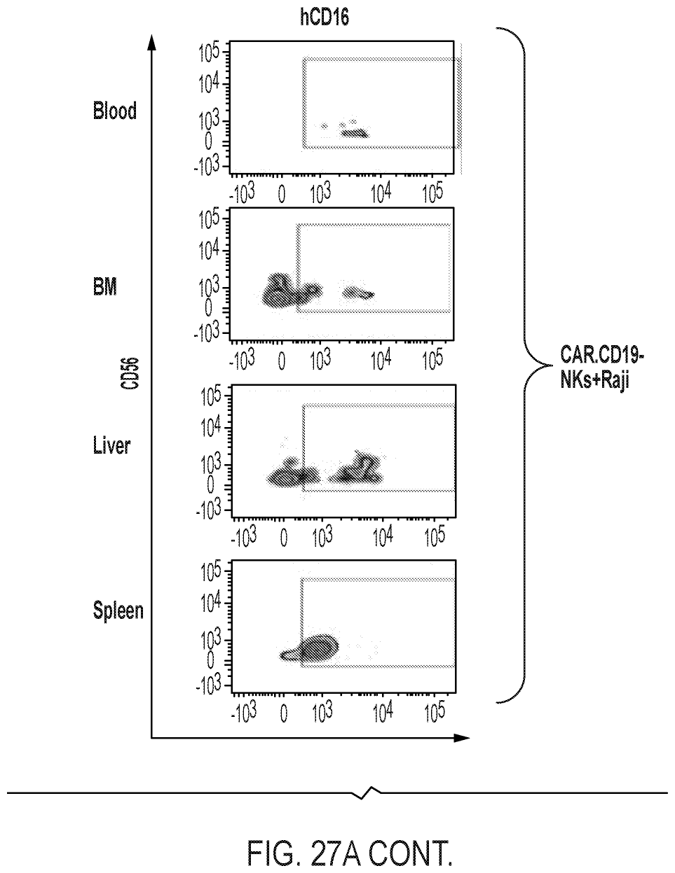

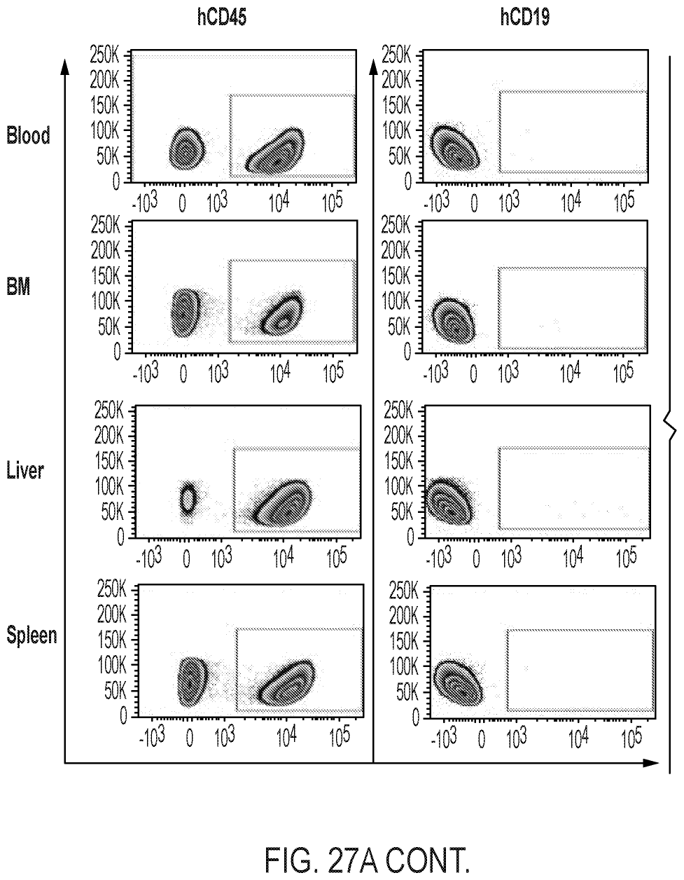

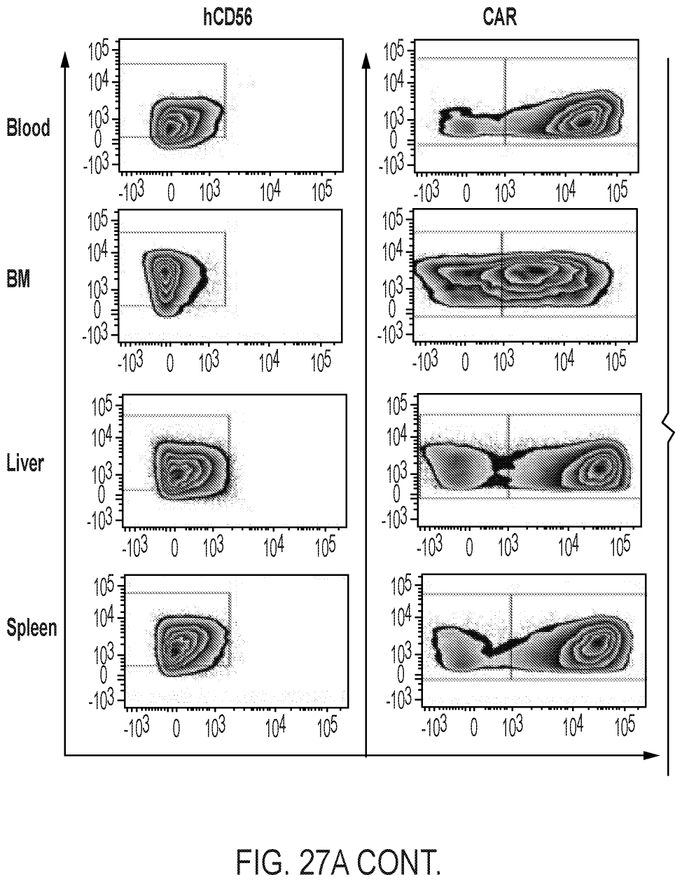

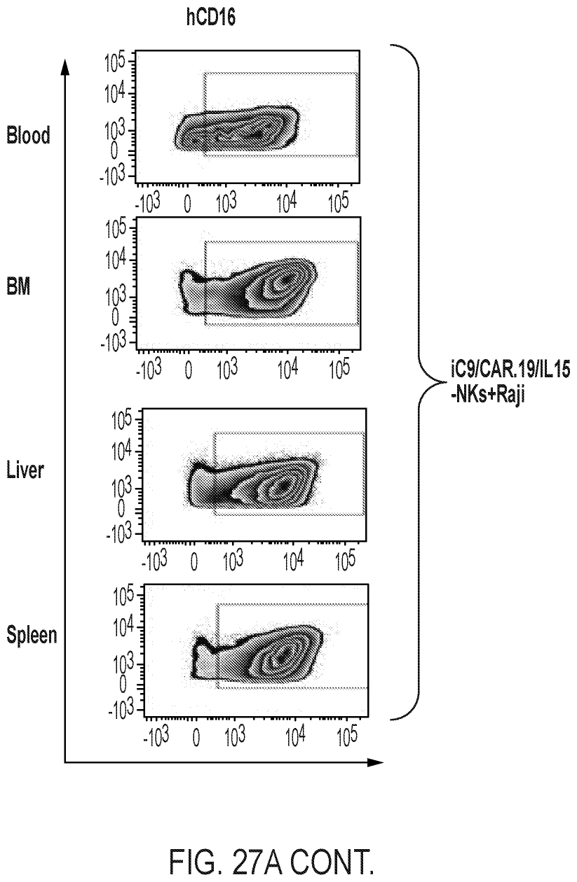

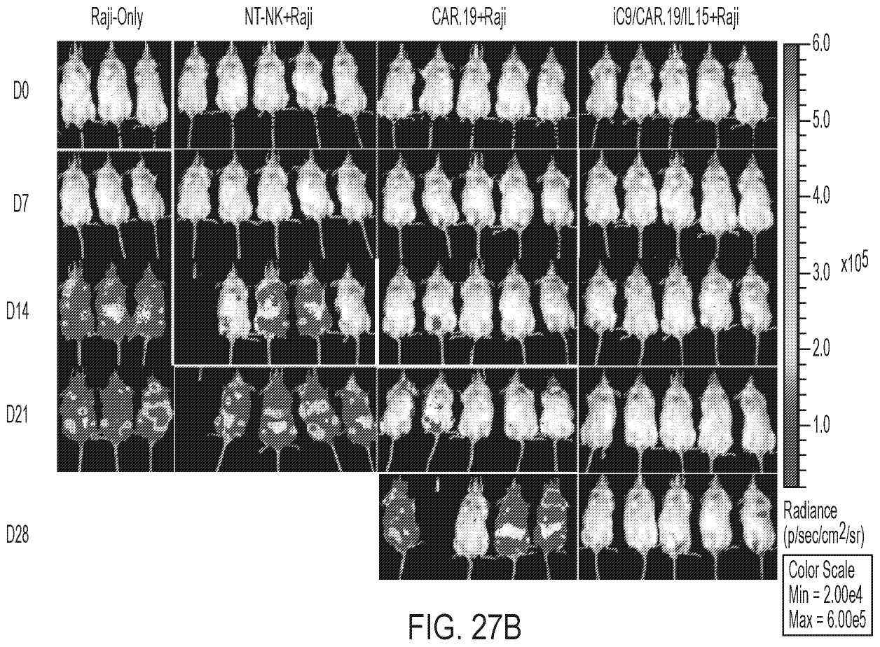

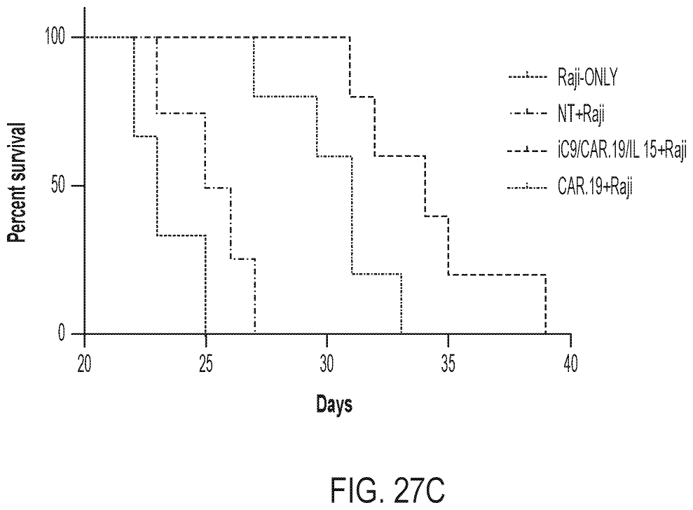

Functional phenotype of ex vivo-expanded CB-NK cells and their cytotoxic activity against myeloid leukemia targets. The expanded CB-NK cells display the full array of activating and inhibitory receptors, continue to strongly express eomesodermin (Eomes) and T-bet (FIG. 26C-26D)(Gill et al., 2012; Intlekofer et al., 2005), two factors necessary for NK cell maturation and activation, lyze myeloid target cells in a dose-dependent manner (FIG. 26E) and upon adoptive transfer into non-obese diabetic severe combined immunodeficient-gamma null (NSG) mice, could home to the bone marrow, liver, spleen and multiple lymphoid tissues (FIG. 27).

Genetic Modification of CB-Derived NK Cells to Enhance their Activity Against Leukemia.

Chimeric antigen receptors (CARs) have been used extensively to redirect the specificity of T cells against leukemia (Sadelain et al., 2003; Rosenberg et al., 2008; June et al., 2009) with dramatic clinical responses in patients with acute lymphoblastic leukemia (ALL) (Brentjens et al., 2013; Kalos et al., 2011; Maude et al., 2015). These infusions have been primarily restricted to the autologous setting because activated T cells from an allogeneic source are likely to increase the risk of GVHD. In this present example one can test the safety and efficacy of engineered CB-derived NK cells, as an alternative to T cells, for the immunotherapy of B-lymphoid malignancies. CB-derived NK cells have multiple potential advantages over T cells: (i) allogeneic NK cells should not cause GVHD, as predicted by observations in murine models, as well as patients with leukemia and solid malignancies treated with haploidentical or CB-derived NK cells (Olson et al., 2010; Rubnitz et al., 2010; Miller et al., 2005); (ii) mature NK cells have a limited life-span of a few weeks, allowing for antitumor activity while reducing the probability of long-term adverse events such as prolonged cytopenias caused by on-target/off-tumor toxicity to normal tissues, or the risk of malignant transformation; (iii) Unlike T-cells, NK cells will also have activity through their native receptors to kill antigen-negative target cells, potentially preventing a mechanism of immune escape; (iv) the generation of an autologous T cell product for each patient is logistically cumbersome and restrictive (Ruggeri et al., 2002; Rubnitz et al., 2010). The use of frozen, off-the-shelf CB units stored in the large global cord blood bank inventory for the generation of NK cells has the potential for widespread scalability that would not be possible with autologous peripheral blood-derived T or NK cell products.

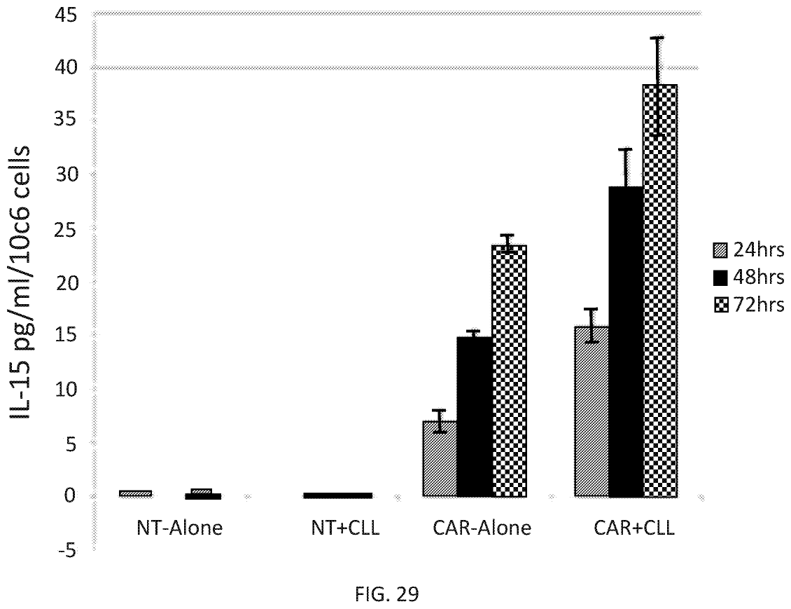

Thus, to improve the persistence and anti-leukemic potency of frozen and ex vivo expanded CBNK cells, the inventors genetically modified them with a retroviral vector, iC9.CAR19-CD28-zeta-2A-IL15 (iC9/CAR.19/IL15), that (i) incorporates the gene for CAR-CD19 to redirect their specificity to CD19; (ii) ectopically produces IL-15, a cytokine crucial for NK cell survival and proliferation (Hoyos et al., 2010; Tagaya et al., 1996), and (iii) expresses a suicide gene, based on inducible caspase-9 (iC9) (Di et al., 2011), that can be pharmacologically activated to eliminate transgenic cells as needed. Initial data show that CB-NK cells can be stably transduced to express the CAR molecule (FIG. 29A). Using a standard 51Cr-release assay, we found that iC9/CAR.19/IL15-transduced CB-NK cells had specific cytotoxic activity against CD19+ Raji cells and primary CLL cells (n=18; FIG. 29B). The NK-CAR and non-transduced NK cells showed equal effector function against K562 cells, indicating that the genetic modification of CB-NK cells did not alter their intrinsic cytotoxicity against NK-sensitive targets (FIG. 29B).

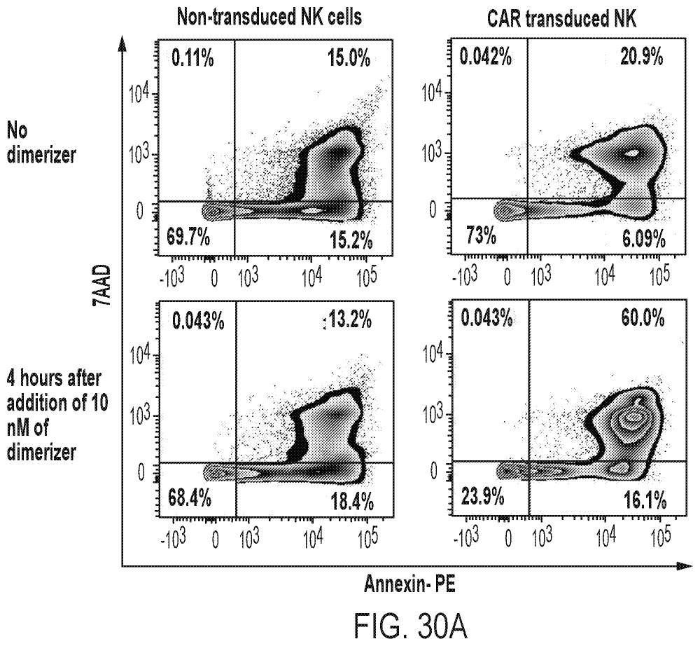

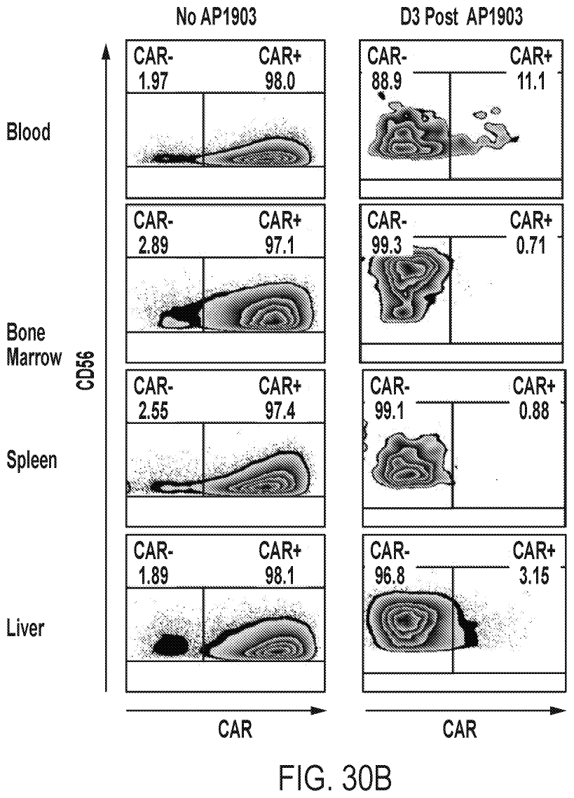

It was next evaluated the trafficking and persistence of iC9/CAR.19/IL15-modified CB-NK cells in vivo, using a NSG mouse Raji xenograft model. NT and iC9/CAR.19/IL15-transduced CB-NK cells were infused in mice engrafted with Raji cells. As shown in FIG. 30A, iC9/CAR.19/IL15+ CBNK cells homed to the spleen, liver and bone marrow (sites of tumor infiltration), while CAR.CD19+ CB-NK cells without the IL-15 gene in the construct, as well as NT CB-NK cells were barely detectable in the tumor sites.

iC9/CAR.19/IL15-tranduced CB-NK cells exert enhanced anti-tumor activity in vivo. To study the in vivo antitumor activity of iC9/CAR.19/IL15-transduced CB-NK cells, we injected NSG mice with FFLuc-labeled Raji cells at 2×10⁵/mouse. On the same day, mice received one 6 i.v infusion of control NT, CAR.19 or iC9/CAR.19/IL15-transduced CB-NK cells (10×10⁶/mouse). Tumor growth was monitored by measuring changes in tumor bioluminescence over time. As shown in FIG. 30B, tumor bioluminescence increased rapidly in mice engrafted with Raji cells and treated with control NT CB-NK cells. By contrast, infusion of either CAR.19+ or iC9/CAR.19/IL15+ CB-NK cells resulted in significant prolongation of survival compared to the effect of NT CB-NK cells (P=0.006 and P=0.001, respectively). Notably, iC9/CAR.19/IL15+ CB-NK cells controlled tumor expansion and prolonged survival (FIG. 30C) significantly better than the CAR.CD19 construct lacking the IL-15 gene, underscoring the important contribution of IL-15 to enhanced antitumor activity.

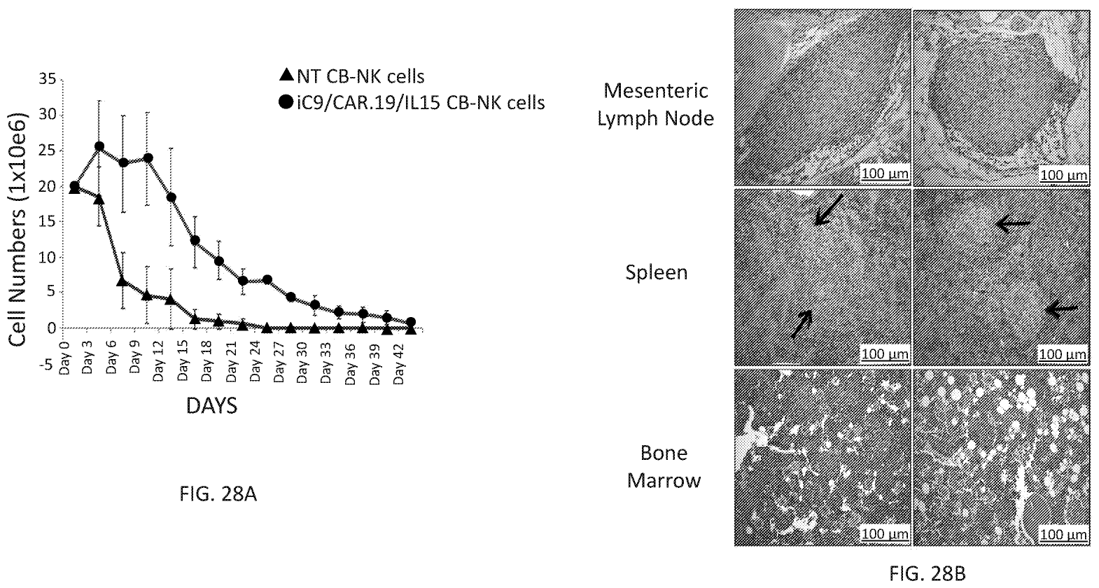

iC9/CAR.19/IL15-transduced CB-NK cells do not show in vitro or in vivo signs of autonomous or dysregulated growth. To investigate the possibility that the IL-15 gene in the vector may result in autonomous or dysregulated growth of transduced CB-NK cells, we cultured iC9/CAR.19/IL15-transduced CB-NK cells in complete Serum-free Stem Cell Growth Medium (SCGM) without the addition of exogenous IL-2 or clone 9.mbIL21 stimulation for 42 days (n=5). Viable cells were enumerated and passaged every three days by replacing media with fresh complete SCGM. As shown in FIG. 28A, the iC9/CAR.19/IL15-transduced CB-NK cell cultures did not show any signs of abnormal growth over 6 weeks, after which, the cells stopped expanding. Karyotyping performed on iC9/CAR.19/IL15-transduced CB NK cells cultured for up to 17 weeks (n=7) failed to detect any chromosomal alterations (data not shown). The inventors also performed chromosome and SNP microarray analyses on paired CB-NK cells (n=6) before (at baseline) and up to 22 weeks after CAR-transduction and ex vivo expansion, and did not observe any evidence of genetic instability. With a follow-up exceeding 10 months, we did not observe any evidence of autonomous growth or leukemic transformation in mice treated with iC9/CAR.19/IL15 or CAR.19-transduced CB-NK cells. Histopathologic examination did not reveal any lymphocytic infiltration, proliferation or lymphoma in any tissue of these mice. The rudimentary lymphoid tissues of the spleen and lymph nodes were free of lymphocytes in all NSG mice from both groups of animals (FIG. 28B), nor was there any lymphocytic infiltration or proliferation in the bone marrow of these mice. Hematologic tests indicated normal numbers of white blood cells and lymphocytes, and no evidence of lymphocytic leukemia in both groups of mice.

IL-15 Production by CAR-Transduced NK Cells

To verify that iC9/CAR.19/IL15+ CB-NK cells can produce IL-15, control NT CB-NK and iC9/CAR.19/IL15+ CB-NK lymphocytes were cultured in triplicates in the presence or absence of CD19+ CLL B cells and culture supernatants were collected to measure IL15 release after 24, 48 and 72 hours of culture. As shown in FIG. 29, IL15 was undetectable in supernatants collected from non-transduced CB-NK cells cultured alone or with CLL targets. By contrast, iC9/CAR.19/IL15+ CB-NK cells produced small amounts of IL15 in the absence of antigen stimulation [average 15.05 pg/mL/106 cells (range 6.2-23.47 pg/mL)], which significantly increased with antigen stimulation [average 27.61 pg/mL/10⁶ cells (range 15.82-38.18 pg/mL)] (P=0.02). The ability was examined of iC9/CAR.19/IL15-transduced NK cells to produce IL-15 in vivo in NSG mice engrafted with Raji cells. Serum levels of IL-15 levels the height of NK cell expansion (2 weeks post expansion) were 40-50 pg/mL, and equivalent to levels detected in the supernatant of cultured cells.

Exogenous recombinant human IL-15 (RhIL-15) has been used in the clinical setting. In a recent phase 1 study in patients with metastatic melanoma or renal cell carcinoma, bolus infusions of 3.0, 1.0, and 0.3 μg/kg per day of IL-15 were administered for 12 consecutive days to patients with metastatic malignant melanoma or metastatic renal cell cancer (Conlon et al., 2015). RhIL-15 was shown to activate NK cells, monocytes, γδ, and CD8 T cells. The 3.0-, 1.0-, and 0.3-μg/kg per day doses resulted in a maximum serum concentration (Cmax) of 43,800±18,300, 15,900±1,900, and 1,260±350 pg/mL, respectively. Dose-limiting toxicities observed in patients receiving 3.0 and 1.0 μg/kg per day were grade 3 hypotension, thrombocytopenia, and elevations of ALT and AST, resulting in 0.3 μg/kg per day being determined the maximum-tolerated dose. There was not observed toxicity releated to IL-15 release by iC9/CAR.19/IL15-transduced CB-NK cells in our clinical study. This is likely because the levels of IL-15 produced by the transduced NK cells are on average 2-3 logs lower than that achieved in the clinical trial of exogenous IL-15 treatment.

iC9/CAR.19/IL15+ CB-NK cells are eliminated after activation of the suicide gene by exposure to a small-molecule dimerizer. To counteract the possibility of excessive toxicity mediated by the release of inflammatory cytokines by transduced CB-NK cells or uncontrolled NK cell growth, we incorporated a suicide gene based on the inducible caspase-9 gene in the construct (Di et al., 2011). As shown in FIG. 30A, the addition of as little as 10 nM of a small molecule dimerizer to cultures of iC9/CAR.19/IL15-transduced CB-NK cells induced apoptosis/necrosis of 60% of transgenic cells within 4 hours as assessed by annexin-V and 7AAD staining but had no effect on the viability of NT CB-NK cells. The suicide gene was also effective in vivo. Mice were engrafted i.v. with Raji tumor cells and treated with iC9/CAR.19/IL15-transduced CB-NK cells. Administration of the small-molecule dimerizer AP1903 (50 i.p. 2 days apart) 10-14 days after NK cells had localized and expanded at different tumor sites later (FIG. 30B, left panel), resulted in a striking reduction in iC9/CAR.19/IL15-transduced CB-NK cells in the blood and tissues of the treated mice (FIG. 30B, right panel), indicative of in vivo elimination of the transgenic cells.

Clinical Trial to Evaluate the Safety and Efficacy of CB-NK Cells Transduced with iC9/CAR.19/IL15 in Patients with Relapsed/Refractory B-Lymphoid Malignancies.

This is a Phase I/II dose-escalation trial to evaluate the safety and relative efficacy of iC9/CAR.19/IL15-transduced CB-NK cells in patients with relapsed/refractory B-lymphoid malignancies (ALL, CLL, NHL). This clinical study will capitalize on the synergistic antitumor activity produced by CAR CB-NK cells and the favorable lymphopenic environment induced by a lymphodepleting regimen (Dudley et al., 2002; Dudley et al., 2005). Thus, patients are treated with cyclophosphamide at a dose 5 of 300 mg/m/day for 3 days. Escalating doses of iC9/CAR.19/IL15-transduced CB-NK cells (10 7/kg-10/kg) are infused once, on day 0, to determine the highest dose at which iC9/CAR.19/IL15-transduced CB-NK cells can be safely infused into patients with relapsed/refractory B-lymphoid malignancies, as defined by standard NCI toxicity criteria. A CB unit matched at 4/6, 5/6, or 6/6 HLA class I (serological) and II (molecular) antigens with the patient are used for CB-NK expansion and CAR transduction. The CB units may be obtained from the MD Anderson cord blood bank.

To gain insight into the persistence, functionality and antileukemic potential of adoptively transferred iC9/CAR.19/IL15-transduced CB-NK cells, one can perform a series of phenotypic and functional assays. One can evaluate the magnitude of expansion and duration of persistence for adoptively infused genetically-modified NK cells in serially acquired PB samples by Q-PCR, using a primer pair that specifically amplifies the unique CAR transgene with sensitivity to detect 1/10,000 CAR+ NK cells. If there are sufficient numbers of circulating NK cells we will quantify by flow cytometry using a mAb specific against the CH2-CH3 region of iC9/CAR.19/IL15 with sensitivity to detect 1/1,000 CAR+ NK cells. The flow cytometry measurements will be coupled with analysis of cell surface NK activating and inhibitory receptor expression. One can evaluate for maintenance of CD19-redirected effector function, using ⁵¹Cr release assay, CD107a degranulation (Rubio et al., 2003; Rezvani et al., 2009), cytokine release (determined by intracellular cytokine assay for IFNγ and IL-2) and chemokine release (MIP1-α and MIP-1β), against CD19-expressing cell lines and, when available, primary CD19+ tumor cells collected and stored from recipients prior to treatment.

To counteract any potential complications (Porter et al., 2011; Grupp et al., 2013), one can incorporate a suicide gene based on the inducible caspase-9 gene (for example) into the CAR19 vector (Hoyos et al., 2010). As shown in FIG. 30, the addition of a small molecule dimerizer, AP1903, induces rapid apoptosis of transgenic cells, such that in the case of prolonged B lymphopenia, the dimerizer could be introduced to induce apoptosis of CAR19-transduced CB-NK cells, allowing normal recovery of B cells. This strategy would also be useful if the transduced NK cells are found to induce GVHD.

Example of Patient Eligibility

Inclusion Criteria:

1. Patients with history of CD19 positive B-lymphoid malignancies (ALL, CLL, NHL) who have received at least 2 lines of standard chemoimmunotherapy or targeted therapy and have persistent disease.

2. Patients with ALL, CLL, NHL with relapsed disease following standard therapy or a stem cell transplant.

3. Patients at least 3 weeks from last cytotoxic chemotherapy at the time of starting lymphodepleting chemotherapy. Patients may continue tyrosine kinase inhibitors or other targeted therapies until at least two weeks prior to administration of lymphodepleting chemotherapy.

4. Karnofsky/Lansky Performance Scale >70.

5. Adequate organ function:

a. Renal: Creatinine clearance (as estimated by Cockcroft Gault) >1=60 cc/min.

b. Hepatic: ALT/AST </=2.5×ULN or </=5×ULN if documented liver metastases, Total bilirubin </=1.5 mg/dL, except in subjects with Gilbert's Syndrome in whom total bilirubin must be </=3.0 mg/dL.

c. Cardiac: Cardiac ejection fraction >/=50%, no evidence of pericardial effusion as determined by an ECHO or MUGA, and no clinically significant ECG findings.

d. Pulmonary: No clinically significant pleural effusion, baseline oxygen saturation >92% on room air.

6. Able to provide written informed consent.

7. 7-80 years of age.

8. All participants who are able to have children must practice effective birth control while on study. Acceptable forms of birth control for female patients include: hormonal birth control, intrauterine device, diaphragm with spermicide, condom with spermicide, or abstinence, for the length of the study. If the participant is a female and becomes pregnant or suspects pregnancy, she must immediately notify her doctor. If the participant becomes pregnant during this study, she will be taken off this study. Men who are able to have children must use effective birth control while on the study. If the male participant fathers a child or suspects that he has fathered a child while on the study, he must immediately notify his doctor.

9. Signed consent to long-term follow-up protocol PA17-0483.

Exclusion Criteria:

1. Positive beta HCG in female of child-bearing potential defined as not postmenopausal for 24 months or no previous surgical sterilization or lactating females.

2. Known positive serology for HIV.

3. Presence of Grade 3 or greater toxicity from the previous treatment.

4. Presence of fungal, bacterial, viral, or other infection requiring IV antimicrobials for management. Note: Simple UTI and uncomplicated bacterial pharyngitis are permitted if responding to active treatment.

5. Presence of active neurological disorder(s).

6. Concomitant use of other investigational agents.

Example of Treatment Plan

Lymphodepleting Chemotherapy (inpatient):

On or before

D-15 Begin NK cell production

D-6 Admit/IV Hydration

D-5 Fludarabine 30 mg/m² IV/Cyclophosphamide 300 mg/m² IV/

Mesna 300 mg/m² IV

D-4 Fludarabine 30 mg/m² IV/Cyclophosphamide 300 mg/m² IV/

Mesna 300 mg/m² IV

D-3 Fludarabine 30 mg/m² IV/Cyclophosphamide 300 mg/m² IV/

Mesna 300 mg/m² IV

D-2 Rest

D-1 Rest

D0 Infusion of iC9/CAR.19/IL15-transduced CB-NK cells (per dose level)

Between D7 Infusion of iC9/CAR.19/IL15-transduced CB-NK cells (per dose level)*

and D14

Lymphodepleting Chemotherapy (outpatient):

On or before

D-15 Begin NK cell production

D-5 Fludarabine 30 mg/m2 IV/Cyclophosphamide 300 mg/m² IV/Mesna 300 mg/m² IV

D-4 Fludarabine 30 mg/m2 IV/Cyclophosphamide 300 mg/m² IV/Mesna 300 mg/m² IV

D-3 Fludarabine 30 mg/m2 IV/Cyclophosphamide 300 mg/m2 IV/Mesna 300 mg/m² IV

D-2 Rest

D-1 Rest

D0 Infusion of iC9/CAR.19/IL15-transduced CB-NK cells (per dose level)

Between D7 Infusion of iC9/CAR.19/IL15-transduced CB-NK cells (per dose level)* and D14

* If no DLT is observed during the first 7 days following the initial NK cell infusion, the patient may be given a 2nd NK cell infusion between days 7 and 14, using the same NK cell dose given initially.

Three dose levels may be tested: 10E5, 10E6, and 10E7 per kilogram body weight. CAR NK cell infusion is dosed per adjusted body weight for patients weighing >20% above their ideal body weight. For patients less than or equal to 20% above their ideal body weight, the actual body weight is used.

If the patient has relapsed or has persistent disease after a protocol assessment, an additional CAR NK infusion may be given. If there are left over cells from their first production, they may be used or a new cord unit may be selected for CAR NK generation. Prescreening testing will not need to be repeated if within 45 days of the previous tests, or at physician discretion.

Cyclophosphamide is dosed per adjusted body weight for patients weighing >20% above their ideal body weight. For patients less than or equal to 20% above their ideal body weight, the actual body weight is used.

On D0 the NK cell infusion will be administered intravenously. Premedicate with Benadryl 25 mg po or IV and Tylenol 650 mg po. The use of steroids is contraindicated unless required for physiologic replacement.

Subjects may be either inpatient or outpatient for the CAR NK infusion, depending upon bed availability and/or patient's clinical situation. Vital signs (temperature, heart rate, blood pressure, and respiratory rate) will be obtained on all patients per BMT standard of care for cellular therapy.
Start of the CAR NK infusion approximately every 15 minutes×4
Then approximately every 30 minutes×2 or until 1 hour after completion of the CAR NK infusion.
Then approximately every hour as indicated by patient's condition.

NK cells may be obtained by the following method:

Frozen cord blood units may be thawed and mononuclear cells may be isolated by Ficoll density gradient centrifugation. NK cells will be CAR transduced and generated for 14 to 22 days in liquid cultures using APC feeder cells as described in detail in the Chemistry, Manufacturing and Controls (CMC).

NK Product Release Criteria

The following minimum criteria may be required for release of the expanded NK cells for reinfusion:

Stat Gram Stain: “No Organisms Seen”.

CAR+ NK cells: >15%

CD3+ number: <2 e5 CD3+ cells/kg.

CD32+ cell number (aAPV): <5%

NK cells (CD16+/56+): >80%

Visual Inspection: “No Evidence of Contamination” (turbidity; change in media color).

Endotoxin Assay: <5EU/Kg.

Viability: ≥70%.

Other parameters that may be monitored include sterility culture for bacteria and fungi. If more than 2×10⁵ CD3+ cells/kg are present, a second cycle of CD3 depletion may be performed. The cell dose for infusion may be reduced so that the infused CD3+ cells are <2×10⁵/kg. If adequate CAR+ NK cell dose is not generated, then all available cells will be infused. If this occurs for patients in the MTD finding stage of this study, they will not be counted in any cohort. If more than the required NK dose is generated, the additional NK cells may be cryopreserved for future infusions or may be used for research. If CAR NK cells cannot be released due to microbial contamination, another cord blood unit will be selected and production will start over. If the patient has already completed lymphodepleting chemotherapy, they may require a second dose of lymphodepleting chemotherapy prior to the CAR NK infusion.

Frozen cells: CAR NK cells that begin production prior to D-15 may be cryopreserved and released for infusion after meeting release criteria. The cryopreserved cells can be thawed for infusion on D0 per GMP standard operating procedures. Since it has been shown of the safety, efficacy, and in vivo expansion and persistence of freshly infused CAR NK cells in the first 9 patients, one can use a frozen and off-the-shelf CAR NK product and treat an additional 3 patients at the highest dose level of 1×10⁷/kg with a frozen CAR NK product. The safety and efficacy of this approach will be assessed using the effTox approach. One can look for the presence of viable NK cells on approximately days +1, +3 and +7 post-infusion. If the levels are comparable to those we have seen with fresh CAR NK cells, we will proceed to the Phase II portion of the study using frozen CAR NK cells.

Administration of the Dimerizer AP1903 for Cytokine Release Syndrome (CRS), Neurotoxicity, or GVHD.

Steps can be taken to address CRS, neurotoxicity, and GVHD. One can administer Tocilizumab 8 mg/kg IV q 6 h as needed for up to 3 doses/24 h for Grade 2 CRS or Grade 2 Neurotoxicity not responding to standard supportive measures. For Grade 3 CRS and Grade 3 Neurotoxicity, in addition to the Tocilizumab, a single dose of AP1903 may be administered (0.4 mg/kg as an intravenous infusion over approximately 2 hours). The AP1903 dose is based on published Pk data which show plasma concentrations of 10-1275 ng/mL over the 0.01 mg/kg to 1.0 mg/kg dose range 65 with plasma levels falling to 18% and 7% of maximum at 0.5 and 2 hrs post dose. The dimerizer can also be used for the treatment of grades I-IV GVHD. Responses in patients with GVHD who had received Capsase-9+ T cells and then the AP1903, responses have occurred within the first 24-48 hours. Patients who do not experience downgrading or CRS or neurotoxicity to Grade 2 or less within 12 hours may receive a second dose of AP1903 but will also receive high dose steroids.

Evaluation: Any Time Before or During Study: HLA Typing (High Resolution A, B, DR).

The following evaluations may be obtained within 30 days of study enrollment: History and physical examination; CBC w/diff and platelets, total bilirubin, SGPT, alkaline phosphatase, LDH, albumin, total protein, BUN, creatinine, glucose, electrolytes, PT/PTT, type and screen, immunoglobulin levels (IGG, IGM, IGA), and cytokine panel 3 (IL6, IFN gamma, TNF alpha); Serology for HIV; ECHO or MUGA; Pulmonary function tests, if clinically indicated; Chest x-ray; Urinalysis; CT brain; PET/CT scan as clinically indicated; Bone marrow aspiration as clinically indicated; EKG.

Evaluations within 7 Days of Starting Lymphodepleting Chemotherapy:

History and Physical Examination Including Weight and Vital Signs.

Laboratory examinations: CBC w/diff and platelets, total bilirubin, SGPT, alkaline phosphatase, LDH, albumin, total protein, BUN, creatinine, glucose, electrolytes, and cytokine analysis. Serum pregnancy test if female participant of childbearing potential.

The following evaluations may be obtained on day 0, day 3 (+/−1 day), day 7 (+/−2 days), day 14 (+/−2 days), and day 21 (+/−3 days), week 4 (+/−5 days), week 8 (+/−5 days), week 12 (+/−5 days), week 16 (+/−14 days), month 6 (+/−28 days), month 9 (+/−28 days) and 1 year (+/−28 days) after CAR-NK infusion:

Physical examination including weight and vital signs at Day 7 (+/−2 days) only.

CBC w/diff and platelets, chemistry panel, and cytokine analysis.

Cytokine panel 3 (IL6, IFN gamma, TNF alpha) at all time points, except only as clinically indicated at month 6 (+/−28 days), month 9 (+/−28 days), and month 12 (+/−28 days).

HLA antibodies at 4 weeks (+/−5 days) and 12 weeks (+/−5 days) only.

Research Labs: CAR NK Detection, Phenotype and Function

The following evaluations may be obtained on week 4 (+/−5 days), week 8 (+/−5 days), week 12 (+/−5 days), week 16 (+/−14 days), and 6 months (+/−28 days), month 9 (+/−28 days), and month 12 (+/−28 days) after CAR-NK infusion:

PET/CT scan as clinically indicated.

The following evaluations may be obtained on day 7 (+/−2), week 4 (+/−5 days), week 8 (+/−5 days), week 12 (+/−5 days), week 16 (+/−14 days), month 6 (+/−28 days), month 9 (+/−28 days) and 1 year (+/−28 days) after CAR-NK infusion:

Bone marrow aspiration and/or biopsy as clinically indicated.

Research Labs: 5 to 10 mL bone marrow aspirate.

Lymph Node Biopsy

If the patient has a diagnostic lymph node biopsy, a portion of the specimen may be analyzed, if available.

RCR Testing on NK CAR Cells

RCR testing for the NK culture cells is sent out on Day −4 by the GMP lab. If the RCR results come back positive, the NK CAR cells cannot be infused. The patient must then come off study. If the results are delayed, then the NK culture may continue for an additional week.

See Table of Evaluations in FIG. 34. In such case, Time frame windows: Days 3 (+/−1 day), 7 (+/−2 days), 14 (+/−2 days), and 21 (+/−3 days), Weeks 4 (+/−5 days), 8 (+/−5 days), 12 (+/−14 days), 16 (+/−14 days), and Months 6 (+/−28 days), 9 (+/−28 days), and 12 (+/−28 days). 1History & Physical, CBC, chem panel: Within 30 days and 7 days of starting lymphodepleting chemotherapy. Pregnancy test: Within 7 days of starting lymphodepleting chemotherapy. Physical at 2 Day 7 (+/−2 days) only. 3 As clinically indicated. Drawn as part of the research lab z code. Samples will be batched and run approximately every 3 months for results. 4Cytokine Panel 3: Day 0 prior to CAR NK infusion. 5 Drawn as part of protocol LABOO-099. 6Replication-competent retrovirus (RCR): About 1, 3, and 6 months post NK cell infusion, and then once every 6 months for 5 years, and then one a year after that for 10 years, per long term follow up study PA17-0483.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Appelbaum, Leukemia 1997; 11 Suppl 4:S15-S17.
Arora et al. Blood. 2012; 119(1):296.
Austin-Ward and Villaseca, Revista Medica de Chile, 126(7):838-845, 1998.
Ausubel et al., Current Protocols in Molecular Biology, Greene Publishing Associates and John Wiley & Sons, N Y, 1994.
Bader et al. J. Clin. Oncol. 2004; 22:1696-1705.
Bar et al. Leuk. Res. Treatment. 2014; 2014:421723.
Bennekov et al., Mt. Sinai J. Med. 71 (2): 86-93, 2004.
Brentjens et al., Sci. Transl. Med. 2013; 5:177ra38.
Bukowski et al., Clinical Cancer Res., 4(10):2337-2347, 1998.
Burger et al. N. Engl. J. Med. 2015; 373:2425-2437.
Byrd et al. N. Engl. J. Med. 2013; 369:32-42.
Byrd et al. Blood 2015; 125:2497-2506.
Caligiuri, Blood 2008; 112:461-469.
Camacho et al. J Clin Oncology 22(145): Abstract No. 2505 (antibody CP-675206), 2004.
Campbell, Curr. Top. Microbiol. Immunol., 298: 23-57, 2006.
Cheah et al., Ann. Oncol. 2015; 26:1175-1179.
Cheson et al. J. Clin. Oncol. 1990; 8:813-819.
Cheson et al. J Clin Oncol 2014; 32: 3059-68.
Chothia et al., EMBO J. 7:3745, 1988.
Christodoulides et al., Microbiology, 144(Pt 11):3027-3037, 1998.
Cohen et al. J Immunol. 175:5799-5808, 2005.
Coiffier. N. Engl. J. Med. 2002; 346:235-242.
Conlon et al. J. Clin. Oncol. 2015; 33:74-82.
Connors, Mod. Pathol. 2013; 26 Suppl 1:S111-S118.
Davidson et al., J. Immunother 21(5):389-398, 1998.
Davila et al. PLoS ONE 8(4): e61338, 2013.
Denman et al., PLoS. One. 2012; 7:e30264.
Di et al., N. Engl. J. Med. 2011; 365:1673-1683.
Doulatov et al., Cell Stem Cell. 10:120-36, 2012.
Dudley et al. Science 2002; 298:850-854.
Dudley et al. J. Clin. Oncol. 2005; 23:2346-2357.
European patent application number EP2537416
Fedorov et al., Sci. Transl. Medicine, 5(215), 2013.
Fielding et al. Blood 2016-0641 pg. 68 2009; 113:4489-4496.
Frolet et al., BMC Microbiol. 10:190 (2010).
Gaj et al., Trends in Biotechnology 31(7), 397-405, 2013.
Gill et al., Blood. 2012 Jun. 14; 119(24):5758-68.
Gisselbrecht et al. J. Clin. Oncol. 2010; 28:4184-4190.
Gokbuget et al. Blood 2012; 120:1868-1876.
Goldstone et al. Blood 2008; 111:1827-1833.
Gribben, Best. Pract. Res. Clin. Haematol. 2007; 20:513-527.
Grupp et al. N. Engl. Med. 2013; 368:1509-1518.
Hallek et al. Blood 2008; 111(12):5446-5456.
Hallek et al. Blood 2018; 131: 2745-60.
Hamadani et al., Biol. Blood Marrow Transplant. 2013; 19:625-631.
Hanibuchi et al., Int. J. Cancer, 78(4):480-485, 1998.
Heemskerk et al. Hum Gene Ther. 19:496-510, 2008.
Hellstrand et al., Acta Oncologica, 37(4):347-353, 1998.
Hollander, Front. Immun., 3:3, 2012.
Honigberg et al. Proc. Natl. Acad. Sci. U.S.A 2010; 107:13075-13080.
Hoyos et al. Leukemia 2010; 24: 1160-70.
Hubert et al., Proc. Natl. Acad. Sci. USA 96 14523-28, 1999.
Hui and Hashimoto, Infection Immun., 66(11):5329-5336, 1998.
Hurwitz et al. Proc Natl Acad Sci USA 95(17): 10067-10071, 1998.
Ibrahim et al., Bayesian Survival Analysis. Springer, N Y, 2001. 2015.
International Patent Publication No. WO 00/37504
International Patent Publication No. WO 01/14424
International Patent Publication No. WO 2007/069666
International Patent Publication No. WO 2007/069666
International Patent Publication No. WO 98/42752
International Patent Publication No. WO/2014055668
International Patent Publication No. WO1995001994
International Patent Publication No. WO1998042752
International Patent Publication No. WO2000037504
International Patent Publication No. WO200014257
International Patent Publication No. WO2001014424
International Patent Publication No. WO2006/121168
International Patent Publication No. WO2007/103009
International Patent Publication No. WO2009/101611
International Patent Publication No. WO2009/114335
International Patent Publication No. WO2010/027827
International Patent Publication No. WO2011/066342
International Patent Publication No. WO2012/129514
International Patent Publication No. WO2013/071154
International Patent Publication No. WO2013/123061
International Patent Publication No. WO2013/166321
International Patent Publication No. WO2013126726
International Patent Publication No. WO2014/055668
International Patent Publication No. WO2014031687
International Patent Publication No. WO2015016718
International Patent Publication No. WO99/40188
Intlekofer et al., Nat. Immunol. 2005; 6:1236-1244.
Iuliucci, et al. Journal of Clinical Pharmacology 41, 870-879 (2001).
Jain et al. Blood 2015; 125:2062-2067.
Janeway et al, Immunobiology: The Immune System in Health and Disease, 3^{rd} Ed., Current Biology Publications, p. 433, 1997.
Johnson et al. Lancet 1996; 347:1432-1438.
Johnson et al. Blood 114:535-46, 2009.
Jores et al., PNAS U.S.A. 87:9138, 1990.
June et al., Nat. Rev. Immunol. 2009; 9:704-716.
Kalos et al., Sci. Transl. Med. 2011; 3:95ra73.
Kaplan, E L and Meier, P, J. American Statistical Association 53:457-481, 1958. 2015.
Kataoka et al. J Immunol 1996; 156(10):3678.
Keating et al. J. Clin. Oncol. 2005; 23:4079-4088.
Kim et al., Nature Biotechnology 31, 251-258, 2013.
Kirchmaier and Sugden, J Virol., 72(6):4657-4666, 1998.
Krebs. Blood 2009; 113:6593-6602.
Kuruvilla et al. Leuk. Lymphoma 2008; 49:1329-1336.
Lanier L L. Nat. Immunol. 2016-0641 pg. 70, 2008; 9:495-502.
Leal, M., Ann N Y Acad Sci 1321, 41-54, 2014.
Lefranc et al., Dev. Comp. Immunol. 27:55, 2003.
Leung et al. Blood 2012; 120:468-472.
Li et al. Nat Biotechnol. 23:349-354, 2005.
Li et al. Proc. Natl. Acad. Sci. USA 89:4275-4279, 1992.
Linnemann, C. et al. Nat Med 21, 81-85, 2015.
Liu et al. Leukemia 2018; 32(2):520-531.
Ljunggren et al., Immunol. Today 1990; 11:237-244.
Lockey et al., Front. Biosci. 13:5916-27, 2008.
Loewendorf et al., J Intern. Med. 267(5):483-501, 2010.
Ludwig et al. Nature Biotech., (2):185-187, 2006a.
Ludwig et al. Nature Methods, 3(8):637-646, 2006b.
Marschall et al., Future Microbiol. 4:731-42, 2009.
Martin-Fontecha et al. Nat. Immunol. 2004; 5:1260-1265.
Maude et al. N Engl J Med; 371: 1507-17, 2014.
Maude et al., Blood 2015; 125:4017-4023.
Mehta R S, Rezvani K. Hematology Am Soc Hematol Educ Program 2016; 2016: 106-18.
Miller et al. Blood 2005; 105:3051-3057.
Mokyr et al. Cancer Res 58:5301-5304, 1998.
Muftuoglu et al. N Engl J Med 2018; 379: 1443-51.
Neelapu et al. N Engl J Med 2017; 377: 2531-44.
Notta et al., Science, 218-221, 2011.
Olson et al., Blood 2010; 115:4293-4301.
Pardoll, Nat Rev Cancer, 12(4): 252-64, 2012
Parkhurst et al. Clin Cancer Res. 15: 169-180, 2009.
Passweg et al. Bone Marrow Transplant. 2016-0641, pg. 69 1998; 21:153-158.
Poon et al. Bone Marrow Transplant. 2013; 48:666-670.
Poon et al. Biol. Blood Marrow Transplant. 2013; 19:1059-1064.
Porter et al., N. Engl. J. Med. 2011; 365:725-733.
Qin et al., Proc. Natl. Acad. Sci. USA, 95(24):14411-14416, 1998.
Rai et al. N. Engl. J. Med. 2000; 343:1750-1757.
Rezvani et al. Blood 2009; 113:2245-2255.
Rieder et al., J. Interferon Cytokine Res. (9):499-509, 2009.
Rosenberg et al., Nat. Rev. Cancer 2008; 8:299-308.
Rubio et al. Nat. Med. 2003; 9:1377-1382.
Rubnitz et al., J. Clin. Oncol. 2010; 28:955-959.
Ruggeri et al. Science 2002; 295:2097-2100.
Rykman, et al., J. Virol. 80(2):710-22, 2006.
Sadelain et al., Nat. Rev. Cancer 2003; 3:35-45.
Sadelain et al., Cancer Discov. 3(4): 388-398, 2013.
Sambrook et al., Molecular Cloning: A Laboratory Manual, 3^{rd} ed., Cold Spring Harbor Press, Cold Spring Harbor, N.Y. 2001.
Savani et al., Blood 2006; 107:1688-1695.
Sehn et al., J Clin Oncol. 2005 Aug. 1; 23(22):5027-33.
Shah et al., PLoS One. 2013 Oct. 18; 8(10):e76781.
Singh et al., Cancer Research, 68:2961-2971, 2008.
Singh et al., Cancer Research, 71:3516-3527, 2011.
Sullivan et al. N. Engl. J. Med. 1989; 320:828-834.
Tagaya et al. Immunity. 1996; 4:329-336.
Takahashi et al., Cell, 126(4):663-76, 2007.
Tam et al., Blood 2009; 113:4144-4152.
Terakura et al. Blood. 1:72-82, 2012.
Thall and Cook J D. Biometrics 2004; 60(3):684-693.
Thall et al. J Biopharm Stat 2006; 16(5):623-638.
Thall et al. Clin Trials 2014; 11(6):657-666.
Turtle et al., Curr. Opin. Immunol., 24(5): 633-39, 2012.
Turtle et al. J Clin Oncol 2017; 35: 3010-20.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 5,994,136
U.S. Pat. No. 6,013,516
U.S. Pat. No. 6,103,470
U.S. Pat. No. 6,207,156
U.S. Pat. No. 6,225,042
U.S. Pat. No. 6,355,479
U.S. Pat. No. 6,362,001
U.S. Pat. No. 6,410,319
U.S. Pat. No. 6,416,998
U.S. Pat. No. 6,544,518
U.S. Pat. No. 6,790,662
U.S. Pat. No. 7,109,304
U.S. Pat. No. 7,442,548
U.S. Pat. No. 7,446,190
U.S. Pat. No. 7,598,364
U.S. Pat. No. 7,989,425
U.S. Pat. No. 8,008,449
U.S. Pat. No. 8,017,114
U.S. Pat. No. 8,058,065
U.S. Pat. No. 8,071,369
U.S. Pat. No. 8,119,129
U.S. Pat. No. 8,129,187
U.S. Pat. No. 8,183,038
U.S. Pat. No. 8,268,620
U.S. Pat. No. 8,329,867
U.S. Pat. No. 8,354,509
U.S. Pat. No. 8,546,140
U.S. Pat. No. 8,691,574
U.S. Pat. No. 8,735,553
U.S. Pat. No. 8,741,648
U.S. Pat. No. 8,900,871
U.S. Pat. No. 9,175,268
U.S. Patent Publication No. 2010/0210014
U.S. Patent Publication Ser. No. 12/478,154
U.S. Patent Publication No. 2002131960
U.S. Patent Publication No. 2003/0211603
U.S. Patent Publication No. 2005/0260186
U.S. Patent Publication No. 2006/0104968
U.S. Patent Publication No. 2009/0004142
U.S. Patent Publication No. 2009/0017000
U.S. Patent Publication No. 2009/0246875
U.S. Patent Publication No. 2011/0104125
U.S. Patent Publication No. 2011/0301073
U.S. Patent Publication No. 20110008369
U.S. Patent Publication No. 2012/0276636
U.S. Patent Publication No. 2013/0315884
U.S. Patent Publication No. 20130149337
U.S. Patent Publication No. 2013287748
U.S. Patent Publication No. 2014/0120622
U.S. Patent Publication No. 2014022021
U.S. Patent Publication No. 20140294898
Varela-Rohena et al. Nat Med. 14: 1390-1395, 2008.
Wang et al. J Immunother 35(9):689-701, 2012.
Wang et al. N. Engl. J. Med. 2013; 369:507-516.
Wang et al., Blood 2015; 126:739-745.
Wu et al., Adv. Cancer Res., 90: 127-56, 2003.
Wu et al., Cancer, 18(2): 160-75, 2012.
Yamanaka et al., Cell, 131(5):861-72, 2007.
Yawata et al., Blood 2008; 112:2369-2380.
Yu et al., Science, 318:1917-1920, 2007.
Zhou et al. Clin. Lymphoma Myeloma. Leuk. 2014; 14:319-326.
Zysk et al., Infect. Immun. 68(6):3740-43, 2000.

1. An ex vivo method for producing natural killer (NK) cells engineered to express one or more chimeric antigen receptors (CAR) and/or one or more T cell receptors (TCR), comprising:
(a) culturing a starting population of NK cells in the presence of artificial presenting cells (APCs) and at least one cytokine;
(b) introducing one or more CAR and/or TCR expression vectors into the NK cells; and
(c) expanding the NK cells in a gas-permeable bioreactor in the presence of APCs and at least one cytokine, thereby obtaining an expanded population of engineered NK cells.

2. The method of claim 1, wherein the gas permeable bioreactor is G-Rex100M.

3. The method of claim 2, wherein the method does not comprise removal or addition of any media components during step (c).

4. The method of claim 1, wherein the method does not comprise performing HLA matching.

5. The method of claim 1, wherein the engineered NK cells express a CAR and/or a TCR.

6. (canceled)

7. (canceled)

8. The method of claim 1, wherein the starting population of NK cells is obtained from cord blood, peripheral blood, bone marrow, CD34⁺ cells, induced pluripotent stem cells (iPSCs), or an NK cell line.

9. (canceled)

10. The method of claim 8, wherein the cord blood has previously been frozen.

11. The method of claim 8, wherein the cord blood has not previously been frozen.

12. The method of claim 8, wherein the cord blood has been obtained from a healthy donor.

13. The method of claim 8, wherein the starting population of NK cells is obtained by isolating mononuclear cells using a ficoll-paque density gradient.

14. The method of claim 13, further comprising depleting the mononuclear cells of CD3, CD14, and/or CD19 cells to obtain the starting population of NK cells.

15. (canceled)

16. (canceled)

17. The method of claim 1, wherein the APCs are gamma-irradiated APCs.

18. The method of claim 1, wherein the APCs are universal APCs (uAPCs).

19. The method of claim 18, wherein the uAPCs are engineered to express (1) CD48 and/or CS1 (CD319), (2) membrane-bound interleukin-21 (mbIL-21), and (3) 41BB ligand (41BBL).

20. The method of claim 1, wherein the NK cells and APCs are present at a 1:1 to 1:10 ratio.

21. The method of claim 1, wherein the NK cells and APCs are present at a 1:2 ratio.

22. The method of claim 1, wherein the at least one cytokine is IL-2, IL-7, IL-12, IL-21, IL-15, or IL-18.

23. (canceled)

24. The method of claim 1, wherein the culturing and/or expanding of the NK cells is in the presence of 2, 3, or 4 cytokines.

25. The method of claim 24, wherein the cytokines are selected from the group consisting of IL-2, IL-7, IL-12, IL-21, IL-15, and IL-18.

26. (canceled)

27. (canceled)

28. (canceled)

29. (canceled)

30. (canceled)

31. (canceled)

32. (canceled)

33. (canceled)

34. The method of claim 1, wherein steps (a)-(c) are performed in less than 2 weeks.

35. The method claim 1, wherein the NK cells are allogeneic or autologous.

36. (canceled)

37. The method of claim 1, wherein the CAR and/or TCR has antigenic specificity for CD70, BCMA, CD5, CD33, CD47, CD99, CLL1, CD38, U5snRNP200, CD200, BAFF-R, CD19, CD319/CS1, ROR1, CD20, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23, CD30, CD56, c-Met, mesothelin, GD3, HERV-K, IL-11Ralpha, kappa chain, lambda chain, CSPG4, ERBB2, WT-1, EGFRvIII, TRAIL/DR4, VEGFR2, or a combination thereof.

38. The method of claim 1, wherein the expression construct further expresses a cytokine.

39. The method of claim 38, wherein the cytokine is IL-15, IL-21, or IL-2.

40. (canceled)

41. (canceled)

42. A pharmaceutical composition comprising a population of engineered NK cells produced according to the method of claim 1, and a pharmaceutically acceptable carrier.

43. (canceled)

44. (canceled)

45. A method of treating an immune-related disorder in a subject comprising administering an effective amount of engineered NK cells of claim 1 to the subject.

46. The method of claim 45, wherein the method does not comprise performing HLA matching between the subject and donor.

47. The method of claim 45, wherein the NK cells are KIR-ligand mismatched between the subject and donor.

48. The method of claim 45, wherein the absence of HLA matching does not result in graft versus host disease or toxicity.

49. The method of claim 45, wherein the immune-related disorder is a cancer, autoimmune disorder, graft versus host disease, allograft rejection, or inflammatory condition.

50. The method of claim 45, wherein the immune-related disorder is an inflammatory condition and the immune cells have essentially no expression of glucocorticoid receptor.

51. (canceled)

52. (canceled)

53. (canceled)

54. The method of claim 45, wherein the immune-related disorder is a cancer.

55. The method of claim 54, wherein the cancer is a solid cancer or a hematologic malignancy.

56. (canceled)

57. (canceled)

58. (canceled)

59. (canceled)

60. (canceled)

61. (canceled)

62. (canceled)

63. (canceled)

64. (canceled)

65. (canceled)

66. (canceled)


SEQUENCE LISTING (9 sequences)

SEQ ID NO: 1 - DNA, 18 bp, Artificial Sequence
  Description of Artificial Sequence Synthetic oligonucleotide
tgctgttgaggagctgga

SEQ ID NO: 2 - DNA, 18 bp, Artificial Sequence
  Description of Artificial Sequence Synthetic oligonucleotide
agcacaccaggcagagtt

SEQ ID NO: 3 - DNA, 18 bp, Artificial Sequence
  Description of Artificial Sequence Synthetic oligonucleotide
cggctgaggagcggaaga

SEQ ID NO: 4 - DNA, 18 bp, Artificial Sequence
  Description of Artificial Sequence Synthetic oligonucleotide
tggaggtgagcaatcccc

SEQ ID NO: 5 - DNA, 20 bp, Artificial Sequence
  Description of Artificial Sequence Synthetic oligonucleotide
ttaatacgactcactatagg

SEQ ID NO: 6 - DNA, 20 bp, Artificial Sequence
  Description of Artificial Sequence Synthetic oligonucleotide
gttttagagctagaaatagc

SEQ ID NO: 7 - DNA, 27 bp, Artificial Sequence
  Description of Artificial Sequence Synthetic primer
gaacagattattctctcaccattagca

SEQ ID NO: 8 - DNA, 22 bp, Artificial Sequence
  Description of Artificial Sequence Synthetic primer
agcgtattaccctgttggcaaa

SEQ ID NO: 9 - DNA, 14 bp, Artificial Sequence
  Description of Artificial Sequence Synthetic probe
cctggagcaagaag